(19) United States
(12) Patent Application Publication
(10) Pub. No.: US 2026/0000766 A1
(43) Pub. Date: Jan. 1, 2026

(54) ULTRASOUND OPTOGENETIC COMPOSITIONS AND METHODS

(71) Applicants: BOARD OF REGENTS, THE UNIVERSITY OF TEXAS SYSTEM, Austin, TX (US); THE BOARD OF TRUSTEES OF THE LELAND STANFORD JUNIOR UNIVERSITY, Stanford, CA (US)

(72) Inventors: Huiliang WANG, Austin, TX (US); Wenliang WANG, Austin, TX (US); Kai Wing Kevin TANG, Austin, TX (US); Guosong HONG, Stanford, CA (US); Xiang WU, Stanford, CA (US)

(21) Appl. No.: 18/873,412

(22) PCT Filed: Jun. 9, 2023
(86) PCT No.: PCT/US2023/068226

§ 371 Date: Dec. 10, 2024

Related U.S. Application Data
(60) Provisional application No. 63/351,270, filed on Jun. 10, 2022.

(51) Int. Cl. A61K 41/00

(52) U.S. Cl. CPC A61K 41/0033; A61K 41/0057

(57) ABSTRACT

Compositions and methods for ultrasound-induced light generation in the brain (e.g, for use in optogenetic methods) are provided. Mechanoluminescent nanoparticles and liposomes are provided. In some aspects, biocompatible and biodegradable liposomes comprising a sonosensitizer (e.g., IR780) and a chemiluminescent compound (e.g., L012) are provided and can be used, e.g., in combination with focused ultrasound (FUS) to generate light in the brain. Related in vivo and therapeutic methods are also provided.

This application claims the benefit of priority to U.S. Provisional Application No. 63/351,270, filed on Jun. 10, 2022, the entire contents of which are hereby incorporated by reference.

This invention was made with government support under Grant no. K01 MH117490 and Grant no. R35 GM147408 awarded by the National Institutes of Health. The government has certain rights in the invention.

### BACKGROUND OF THE INVENTION

### 1. Field of the Invention

The present invention relates generally to the field of molecular biology and medicine. More particularly, it concerns ultrasound optogenetic compositions, such as liposomes or nanoparticles, and related methods.

### 2. Description of Related Art

Although optogenetics has revolutionized neuroscience understanding by allowing spatiotemporal control over cell-type specific neurons in neural circuits, significant problems have hampered its utility. Over the past decade, optogenetics has become an increasingly important technology for neuroscience research and the treatment of neurological disorders, including Parkinson's disease (PD) and Alzheimer's disease (AD), by providing neuroscientists with precise spatiotemporal control of neural activity with neuron-subtype specificity (Deisseroth, 2015; Fougère et. al., 2021; Etter et. al., 2019). However, due to the poor tissue penetration of light in the activation spectra (430˜610 nm) of conventional opsins, the delivery of light to brain areas requires an invasive partial removal of skull and the implantation of an optical fiber which could damage brain tissue and increase infection risk (Deisseroth, 2015). In addition, perturbation of glial and astrocytic activity and ischemia have been reported during optogenetics due to the intracranial implantation of the device (Won et. al., 2020; Salatino et. al., 2017).

Efforts have been made to use light that can more effectively penetrate into the brain with optogenetic methods, but these approaches have yielded only limited improvements and are unsuitable for use in larger mammals. Several less invasive strategies have been tested, including using specific ChRmine opsins with red-shifted activation spectra (Marshel et. al., 2019; Chen et. al., 2020), two-photon stimulation (Packer et. al., 2012), or upconversion nanoparticles to convert tissue-penetrating near-infrared (NIR) light into visible light after intracortical injection (Chen et. al., 2018). Despite these efforts, the penetration depth using red or NIR light is still insufficient for non-invasive deep brain optogenetic stimulation, especially for large animals and human applications.

Focused ultrasound (FUS) represents one form of wireless energy harvesting strategy that has been recently developed for non-invasive brain local anesthesia, sonodynamic therapy, and chemogenetics for brain stimulation due to its promising tissue penetration depth exceeding 10 cm and biosafety (Choi et. al., 2020; Rwei et. al., 2017; Pan et. al., 2018; Wang et. al., 2018). Experiments have been performed using zinc sulfide nanoparticles co-doped with silver and cobalt (ZnS: Ag,Co@ZnS) to convert ultrasound wave to light for optogenetic stimulation after vein injection of nanoparticles (Wu et. al., 2019; Hong, 2020). However, ZnS: Ag,Co@ZnS nanoparticles were required to be charged with 400 nm light outside the brain before use, and these non-biodegradable inorganic nanoparticles may generally undergo bioaccumulation and bioaugmentation of heavy metals in organs (Kahlon et. al., 2018; Ahamed et. al., 2019; Soenen et. al., 2011). These limitations present significant limitations for clinical use. Thus, the development of biodegradable organic nanoparticles to achieve non-invasive sono-optogenetic stimulation in a simple manner remains a challenge.

Current methodologies for optogenetics that utilize light fibers result in brain damage due to surgical positioning of the fibers into the brain. Visible light cannot be directly delivered to deep brain tissue, due to the severe dissipation and scattering of photons. As a result, invasive craniotomy is usually required to implant optical fibers in the brain for in vivo optogenetic stimulation, resulting in permanent damage and chronic gliosis in brain tissue. Clearly there exists a need for improved methods for light induction in optogenetic methods.

### SUMMARY OF THE INVENTION

The present disclosure overcomes limitations in the prior art by providing compositions and methods for ultrasound-induced optogenetics, including biocompatible biodegradable organic nanoparticles or liposomes that can generate light in the brain and be used in optogenetic methods. In some aspects, the compositions and methods provided herein provide significant improvements over use of non-biodegradable inorganic nanoparticles that can result in bioaccumulation and bioaugmentation in the organs of the subject; for example, select organic nanoparticles or liposomes are provided herein that are biodegradable and do not contain metals or inorganic components that are associated with clinical disadvantages. Additionally, in contrast to previous inorganic nanoparticles, organic nanoparticles and liposomes are provided herein that do not require charging of the nanoparticles prior to administration. In contrast to methods that require the implantation of optical fibers into the brain that results in brain damage and gliosis, compositions and methods are provided herein that allow for ultrasound to be applied to the brain to generate light in the brain for optogenetics. In some aspects, nanoparticles or liposomes are provided that comprise both a sonosensitizer and a chemiluminescent compound. After administering the nanoparticles or liposomes to a mammalian subject, ultrasound (e.g., focused ultrasound or FUS) can be applied to the brain to stimulate or trigger the sonosensitizer to stimulate a chemiluminescent compound to produce light in the brain (e.g., ultrasound can cause the sonosensitizer to generate free radicals or reactive oxygen species that can stimulate the chemiluminescent compound to produce light). This light can be used to stimulate or activate light-sensitive brain receptors (e.g., opsins, channelrhodopsin-2 receptors) used in optogenetic methods in vivo. Further, it has been observed that by including a peroxide (e.g., calcium peroxide) in select nanoparticles described herein, additional sensitivity and stimulation of deeper tissues in the brain by light can be achieved. Nanoparticles may comprise a polymer such as a polyethylene glycol (e.g., PEG-200) on the surface of the nanoparticle that increases the stability of a nanoparticle, e.g., prior to mechanical stimulation causing the release of the sonosensitizer (e.g., IR780, optionally in combination with a peroxide such as calcium peroxide). Related ultrasound-induced optogenetic methods or “sono-optogenetics” are also provided.

As shown in the below examples, to achieve non-invasive optogenetics with high temporal resolution and excellent biocompatibility, focused ultrasound triggered nanoscopic light sources, such as liposomes containing IR780 and L012 (Lipo@IR780/L012), were produced and can be used for deep brain photon delivery. Synchronized and stable blue light emission was generated under FUS irradiation due to the activation of chemiluminescent L012 via nearby reactive oxygen species (ROS) generated by IR780. In vitro tests revealed that Lipo@IR780/L012 could be triggered by FUS for light emission at different frequencies and hence activate opsin-expressing spiking HEK cells under the FUS irradiation. In vivo optogenetic stimulation further demonstrated that motor cortex neurons could be noninvasively and reversibly activated under the repetitive FUS stimulation after intravenous (i.v.) injection of lipid nanoparticles to achieve limb motions control. Although IR780 has been previously used to treat cancers due to ROS generation near tumors, it was surprisingly observed in the below examples that nanoparticles containing IR780 and a chemiluminescent compound (L012) could be used to generate light in the brain sufficient to stimulate opsin receptors in optogenetic methods, without resulting in significant toxicity or causing significant cell death. In contrast to previous inorganic nanoparticles, liposomes are provided herein (e.g., Lipo@IR780/L012) that were observed to be biocompatible and biodegradable and able to stimulate opsins in optogenetic approaches in vivo.

The in vitro tests demonstrated the opsins are effectively activated under the ultrasound irradiation with Lipo@IR780/L012 nanoparticles. Following this, the inventors investigated whether the Lipo@IR780/L012 nanoparticles allowed for noninvasive optogenetic brain stimulation in ChR2-expressing mice after tail vein administration under FUS irradiation. Thyl-ChR2-YFP transgenic mice with ChR2 expressing neurons were used for in vivo sono-optogenetic stimulation. As shown in FIG. 5a, the mouse was head-fixed in a stereotaxic frame and anesthetized with 2.5% isoflurane. Then, Lipo@IR780/L012 nanoparticles at a concentration of 10 mg/mL were injected through the tail vein. The FUS transducer water balloon was placed in direct contact with the intact scalp of the mouse with filling ultrasound gel (FIG. 5a-i and 5a-ii). To visually evaluate sono-optogenetic brain stimulation, the motor cortex areas were irradiated (FIG. 5a-iii), since the motor cortex is responsible for controlling the execution of body movement, including the complex movements of the leg and fingers, allowing us to easily evaluate activation by tracking mouse movement (Sanes and Donoghue, 2000). After i.v. administration off 15 minutes, the isoflurane concentration was decreased to 0.5% to make sure the mice were in light anesthesia status in order to effectively observe its response to FUS irradiation (1.5 MHz, pulse 100 ms on 900 ms off, 6.2 MPa). Camera video was used to track the synchronized limbs' response under the sono-optogenetic stimulations, where the hip, knee and feet were marked with different color dots (FIG. 5b). Kinematic data were obtained by using DeepLabCut to quantify the joint angle (Hip to knee: θ, and knee to feet: φ, shown in FIG. 5b ii) changes under the ultrasound irradiation (Figure S8). Our results revealed significant changes in θ in the Lipo@IR780/L012 nanoparticles (+) group with FUS irradiation, but no change in all other groups in left and right limbs (FIG. 5c and Figure S9). Similar results were also observed in knee to feet movement (φ), indicating the temporary and reversible activation of motor cortex neurons under the repetitive sonoluminescence irradiation. The quantitative analysis of θ (FIG. 5e) and φ (FIG. 5f) angle changes showed that there were statistically significant differences between Lipo@IR780/L012 nanoparticles (+)/FUS irradiation (+) groups and other control groups with Lipo@IR780/L012 nanoparticles (+)/FUS irradiation (−), Lipo@IR780/L012 nanoparticles (−)/FUS irradiation (+) and Lipo@IR780/L012 nanoparticles (−)/FUS irradiation (−). These results revealed that our FUS induced Lipo@IR780/L012 system can be used to achieve highly reliable noninvasive brain stimulation.

These Lipo@IR780/L012 biocompatible liposomal nano-light source triggered by FUS were used for sono-optogenetics and achieved effective activation of motor cortex neurons. Sono-optogenetics achieved the activation of neurons in shallow brain regions due to the limited power density of light from these mechanoluminescence nanoparticles at the biosafe ultrasound power range. The design of mechanoluminescent nanoparticles with high ultrasound sensitivity for light emission is critical for the application of sono-optogenetics in deeper tissues. In order to achieve stimulation of deeper brain regions by the nanoparticles, the inventors discovered that inclusion of a peroxide (e.g., calcium peroxide) in the nanoparticles, optionally with a polyethylene glycol (PEG-200) in the surface of the nanoparticles could be used to increase the sensitivity of the nanoparticles and achieve stimulation of tissues in deeper brain regions. For example, as shown in FIG. 17d and Figure S5. Lipo@IR780/L012/CaO₂ liposomes exhibited higher photon productivity and ultrasound sensitivity, and photon productivity was not observed to have nay evident decrease even within a tissue depth of 8 mm (FIG. 17f and Figure S6). These data demonstrate that nanoparticles provided herein can be used to achieve remote and wireless photon delivery for minimally invasive brain modulation.

As shown in the below examples, the inventors developed additional approaches to increase sensitivity of mechanoluminescent nanoparticles and to enable sono-optogenetic deep brain stimulation. Self-amplifying sono-optogenetics with high ultrasound sensitivity and spatiotemporal resolution were produced and achieved temporal activation of neurons at both the superficial motor cortex and the deep brain VTA (FIG. 15a). Chemiluminescence L012, sonosensitizer IR780, and sono-amplifier polyethylene glycol (PEG) 200 coated calcium peroxide (CaO₂) nanoparticles were loaded into lipids to prepare a nano light transducer for opsin activation under the FUS stimulation. Free radicals generated by IR780 after absorbing ultrasound energy can activate L012 to emit blue light. Meanwhile, the alternating ultrasound pressure wave can perturb the liposome membrane and PEG coating at the surface of CaO₂, thus enabling the reaction of CaO₂ and water to produce hydrogen peroxide (H₂O₂) and calcium hydroxide (Ca(OH)₂), increasing the local concentration of free radicals and pH. Without wishing to be bound by any theory, it is anticipated that L012 reactivity may be inhibited by protonation of the luminol molecule at low pH values, and the increased pH and free radical concentration may contribute to the observed improvements in the quantum yield of L012 (FIG. 15b). In vivo light emission and optogenetic neuronal stimulation was evaluated, and low activation latency was observed, even at a depth of 5 mm, hence achieving minimally invasive, spatiotemporal sono-optogenetic control of neuronal activity in the deep mouse brain tissues.

An aspect of the present disclosure relates to a pharmaceutical composition comprising: (i) liposomes or nanoparticles, wherein the liposomes or nanoparticles comprise a sonosensitizer and a chemiluminescent compound; and (ii) an excipient. The nanoparticles may be a biocompatible, biodegradable organic nanoparticles. In some embodiments, the nanoparticle is a lipid nanoparticle. In some preferred embodiments, the sonosensitizer produces free radicals or a reactive oxygen species (ROS) in response to ultrasound stimulation. In some preferred embodiments, the chemiluminescent compound emits light in response to free radicals or a reactive oxygen species (ROS). The reactive oxygen species (ROS) may be singlet oxygen (¹O₂) or hydroxyl radical (·OH). In some embodiments, the sonosensitizer is IR780, or DCPH-P-Na (I), Hematoporphyrin, Zinc protoporphyrin, methylene blue, TiO₂, Chlorin e6. In some embodiments, the sonosensitizer is IR780. In some embodiments, the chemiluminescent compound is L012, a dioxetane, luminol, isoluminol, an imidazopyrazinone, a lophine, or an acridinium. In some embodiments, the chemiluminescent compound is L012. In some embodiments, the sonosensitizer is IR780 and wherein the chemiluminescent compound is L012. In some embodiments, the sonosensitizer is IR780, DCPH-P-Na(I), Hematoporphyrin, Zinc protoporphyrin or methylene blue, TiO₂, Chlorin e6 and wherein the chemiluminescent compound is L012, Dioxetane derivatives, luminol, isoluminol, imidazopyrazinone derivatives, lophine derivatives or acridinium derivatives. The nanoparticle or liposome may be about 10-1000 nm in diameter or about 50-500 nm in diameter. The nanoparticles or liposomes may be administered to the subject via intravenous injection. The liposomes or nanoparticles may comprise a phospholipid, 1, 2-distearoryl-sn-glycero-3-phosphatidyl choline (DSPC), sphingomyelin, phosphatidylcholine (e.g., egg phosphatidylcholine or soy phosphatidylcholine), monosialoganglioside, cholesterol, polyethylene glycol (PEG), PEG-succinyl cysteine (PEG-SC), poly (lactic-co-glycolic acid) (PLGA), dioleoylphosphatidylethanolamine (DOPE), 1,2-dipalmitoyl-sn-glycero-3-phosphocholine (DPPC), cholesterol, 1,2-distearoyl-sn-glycero-3-phosphoethanolamine-N-[amino(polyethylene glycol)] (DSPE-PEG), or choline phosphate. The liposomes or nanoparticles may comprise a polyethylene glycol. The polyethylene glycol may have a molecular weight from 100-5000 Da, more preferably from 100-500 Da or 100-300 Da. In some embodiments, the polyethylene glycol is PEG-100, PEG-150, PEG-200, PEG-250, PEG-300, PEG-350, PEG-400, PEG-450, or PEG-500. In some embodiments, the polyethylene glycol is present in the liposome or nanoparticle in an amount of about 1-10 (w/w) % or about 0.1-5 mol. %. In some embodiments, the liposomes or nanoparticles comprise 1,2-dipalmitoyl-sn-glycero-3-phosphocholine (DPPC), cholesterol, and 1,2-distearoyl-sn-glycero-3-phosphoethanolamine-N-[amino(polyethylene glycol)-2000] (DSPE-PEG₂₀₀₀). The liposomes may be cationic liposomes, stealth liposomes, anionic liposomes, zwitterionic liposomes, or polymer liposomes. The liposomes may be unilamellar liposomes, multilamellar liposomes, or multivesicular liposomes. In some embodiments, the liposomes are unilamellar liposomes. The pharmaceutical composition may be formulated for injection (e.g., intravenous injection). The liposomes or nanoparticles may further comprise a peroxide, DCPH-P-Na(I), hematoporphyrin, zinc protoporphyrin, methylene blue, TiO2, or Chlorin e6. In some embodiments, the liposomes or nanoparticles further comprise a peroxide. In some preferred embodiments, the peroxide is calcium peroxide (CaO₂). The peroxide or calcium peroxide (CaO₂) may be present in an amount of about 1-10 (w/w) %. In some embodiments, the nanoparticle or liposome comprises calcium peroxide (CaO₂) and a polyethylene glycol (PEG). The polyethylene glycol may have a molecular weight of 100-5000 Daltons (Da), preferably 100-500 Da, or 100-300 Da.

Another aspect of the present disclosure relates to a method of contacting a tissue of a subject with light, comprising: applying an ultrasound signal to a nanoparticle or liposome in proximity to the tissue of the subject, wherein the nanoparticle or liposome comprises a photosensitizer and a chemiluminescent compound, and wherein the ultrasound signal causes the photosensitizer to induce the chemiluminescent to emit light that contacts the tissue. The nanoparticle may be a biocompatible, biodegradable organic nanoparticle. In some embodiments, the nanoparticle is a lipid nanoparticle. In some preferred embodiments, the photosensitizer produces free radicals or a reactive oxygen species (ROS) in response to ultrasound stimulation. In some preferred embodiments, the chemiluminescent compound emits light in response to free radicals or a reactive oxygen species (ROS). The reactive oxygen species (ROS) may be singlet oxygen (¹O₂) or hydroxyl radical (·OH). In some embodiments, the sonosensitizer is IR780, or DCPH-P-Na (I), Hematoporphyrin, Zinc protoporphyrin and methylene blue, TiO2, Chlorin e6. In some embodiments, the sonosensitizer is IR780. In some embodiments, the chemiluminescent compound is L012, a dioxetane, luminol, isoluminol, an imidazopyrazinone, a lophine, or an acridinium. In some embodiments, the sonosensitizer is IR780 and wherein the chemiluminescent compound is L012. In some embodiments, the sonosensitizer is IR780, DCPH-P-Na (I), hematoporphyrin, zinc protoporphyrin, methylene blue, TiO₂, Chlorin e6; and wherein the chemiluminescent compound is L012, a dioxetane, luminol, isoluminol, an imidazopyrazinone, a lophine, or an acridinium. The nanoparticle or liposome may be about 10-1000 nm in diameter or about 50-500 nm in diameter. The nanoparticles or liposomes may be administered to the subject via intravenous injection. The liposomes or nanoparticles may comprise a phospholipid, 1,2-distearoryl-sn-glycero-3-phosphatidyl choline (DSPC), sphingomyelin, phosphatidylcholine (e.g., egg phosphatidylcholine or soy phosphatidylcholine), monosialoganglioside, cholesterol, polyethylene glycol (PEG), PEG-succinyl cysteine (PEG-SC), poly(lactic-co-glycolic acid) (PLGA), dioleoylphosphatidylethanolamine (DOPE), 1,2-dipalmitoyl-sn-glycero-3-phosphocholine (DPPC), cholesterol, 1,2-distearoyl-sn-glycero-3-phosphoethanolamine-N-[amino(polyethylene glycol)] (DSPE-PEG),or choline phosphate. The liposomes or nanoparticles may comprise a polyethylene glycol. In some embodiments, the polyethylene glycol has a molecular weight from 100-5000 Da, more preferably from 100-500 Da. In some embodiments, the polyethylene glycol is PEG-100, PEG-150, PEG-200, PEG-250, PEG-300, PEG-350, PEG-400, PEG-450, or PEG-500. The polyethylene glycol may be present in the liposome or nanoparticle in an amount of about 1-10 (w/w) % or about 0.1-5 mol. %. The liposomes or nanoparticles may further comprise a peroxide DCPH-P-Na(I), hematoporphyrin, zinc protoporphyrin, methylene blue, TiO₂, or Chlorin e6. The liposomes or nanoparticles further may comprise a peroxide. In some preferred embodiments, the peroxide is calcium peroxide (CaO₂). The peroxide or calcium peroxide (CaO₂) may be present in an amount of about 1-10 (w/w) %. In some embodiments, the nanoparticle or liposome comprises calcium peroxide (CaO₂) and a polyethylene glycol (PEG). In some embodiments, the polyethylene glycol has a molecular weight of 100-5000 Daltons (Da), preferably 100-500 Da, or 100-300 Da. In some embodiments, the nanoparticle or liposome comprises calcium peroxide (CaO₂) and a polyethylene glycol (PEG). In some embodiments, the liposomes or nanoparticles comprise 1,2-dipalmitoyl-sn-glycero-3-phosphocholine (DPPC), cholesterol, and 1,2-distearoyl-sn-glycero-3-phosphoethanolamine-N-[amino(polyethylene glycol)-2000] (DSPE-PEG₂₀₀₀). In some embodiments, the liposomes are cationic liposomes, stealth liposomes, anionic liposomes, zwitterionic liposomes, or polymer liposomes. The liposomes may be unilamellar liposomes, multilamellar liposomes, or multivesicular liposomes. In some embodiments, the liposomes are unilamellar liposomes. The tissue may comprise a neuron that comprises a photosensitive protein, and wherein the emitted light modulates the photosensitive protein. The modulation may result in hyperpolarization of the neuron. The modulation may result in depolarization of the neuron. The method may further comprise genetically modifying the neuron to express the photosensitive protein. The photosensitive protein may be a channelrhodopsin, a Volvox carteri light-activated protein (VChRl), a iC++, a ChRmine, a archaerhodopsin, a leptosphaeria rhodopsin (Mac), a or a halorhodopsin (HPHR). The halorhodopsin may be a halorhodopsin from Natronomonas (NpHR). In some embodiments, the channelrhodopsin is channelrhodopsin-2 (ChR2), ChR2 (H134R), C1V1 (t/t), ChIEF, ChETA, C1V1 (t/t), ChrimsonR, VChR1, Chronos, iChloC, SwiChRca, Phobos, Aurora, or CheRiff. The tissue may comprise a group of compounds that causes genetic modification to the tissue after absorbing the emitted light. The group of compounds may comprise a CRISPR compound and a Cas9 compound. In some embodiments, the ultrasound signal is a focused ultrasound signal (FUS). The ultrasound signal may have a frequency ranging from 150 kHz to 15 MHz. The ultrasound signal may be repeated at a rate ranging from 0.2 repetitions per second to 5 repetitions per second. In some embodiments, the ultrasound signal has a spatial peak pulsed average intensity (ISPPA) at a target neuron ranging from 1 W/cm² to 100 W/cm². In some embodiments, the spatial peak pulsed average intensity at a target neuron ranges from 5 W/cm² to 15 W/cm². In some embodiments, the time interval between the application of the ultrasound signal and the emission of light from the photoexcited mechanoluminescent particle is 9 ms or less. The time interval between the photoexcitation of the mechanoluminescent particles and the application of ultrasound may range from 1 second to 60 minutes. In some embodiments, the subject is a mammal (e.g., a rat, a mouse, a monkey, rabbit, pig, or a human). The human may have a neurological disease or neurological injury. The neurological disease may be epilepsy, Alzheimer's disease (AD), or Parkinson's disease (PD). The neurological injury may be spinal cord injury.

Yet another aspect of the present disclosure relates to a system for contacting a tissue of a subject with light, the system comprising: an ultrasound device configured to apply an ultrasound signal to a nanoparticle or liposome described above or herein in proximity to the tissue, thereby causing the mechanoluminescent particle to emit light that contacts the tissue. In some embodiments, the ultrasound signal is a focused ultrasound signal.

Another aspect of the present disclosure relates to a kit comprising the liposome or nanoparticle described above or herein in a container. In some embodiments, the container is a container for syringe loading or a syringe.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range.

Where the stated range includes one or both of the limits, ranges excluding either or both of those included limits are also included in the invention.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “subject,” “host,” “patient,” and “individual” are used interchangeably herein to refer to any mammalian subject for whom therapy is desired, particularly humans. Other subjects may include cattle, dogs, cats, guinea pigs, rabbits, rats, mice, horses, and so on.

The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

The terms “cell,” and “cells,” and “cell population,” used interchangeably, intend one or more mammalian cells. The term includes progeny of a cell or cell population. Those skilled in the art will recognize that “cells” include progeny of a single cell, and there are variations between the progeny and its original parent cell due to natural, accidental, or deliberate mutation or change.

The terms “in operable combination”, “in operable order”, and “operably linked” refer to a linkage wherein the components so described are in a relationship permitting them to function in their intended manner, for example, a linkage of nucleic acid sequences in such a manner that a nucleic acid molecule capable of directing the transcription of a given gene or the synthesis of desired protein molecule, or a linkage of amino acid sequences in such a manner so that a fusion protein is produced.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

Scheme 1. Schematic of FUS triggered blue light emission from Lipo@IR780/L012. (a) After i.v. injection, Lipo@IR780/L012 nanoparticles circulate in blood and emit blue light to the surrounding neurons for brain modulation under FUS irradiation, diagram created using Biorender.com; (b) the mechanism of light emission of Lipo@IR780/L012 nanoparticles under FUS irradiation.

FIGS. 1a-d: The nanoparticle performance of Lipo@IR780/L012. (a) Illustration of IR780 and L012 loaded liposome, Lipo@IR780/L012; (b) size distribution of blank liposome, Lipo@IR780 and Lipo@IR780/L012 determined via DLS; (c) TEM image of Lipo@IR780/L012; (d) the size stability tests of Lipo@IR780/L012 with/without 10% FBS or under the FUS irradiation, determined by DLS.

FIGS. 2a-g. FUS induced ROS generation of Lipo@IR780 nanoparticles. (a) the reaction mechanism of ROS probes in presence of ROS, (i) DPBF specifically reacts with ¹O₂ to generate DBB, (ii) SA will react in the presence of ·OH to generate dihydroxybenzoic acid; (b) time dependent UV-Vis degradation spectrum of DPBF indicating ¹O₂ was generated via Lipo@IR780 under FUS (1.5 MHz, peak pressure 6.2 MPa); (c) quantification analysis of DPBF decomposition in presence of Lipo@IR780 with and without FUS irradiation (n>3 per group); (d) time dependent UV-Vis degradation spectrum of SA indicating ·OH was generated via Lipo@IR780 under FUS (1.5 MHz, peak pressure 6.2 MPa) (n>3 per group); (e) quantification analysis of SA decomposition in presence of Lipo@IR 780 with and without FUS irradiation (n>3 per group); (f) quantification analysis of DPBF decomposition in presence of Lipo@IR780/L012 with and without FUS irradiation (n>3 per group), indicating no ¹O₂ residues escaped from liposomes; (g) quantification analysis of SA decomposition in presence of Lipo@IR780/L012 with and without FUS irradiation (n>3 per group), indicating no ·OH residues escaped from liposomes. All plots show mean±SEM unless otherwise mentioned.

FIGS. 3a-i. FUS triggered light emission of Lipo@IR780/L012. (a) Mechanoluminescence spectrum of Lipo@IR780/L012 nanoparticles (black) overlaid with ChR2 absorption spectrum (red dot curve) and CheRiff absorption spectrum (blue dot curve); (b) illustration of the FUS induced light emission of Lipo@IR780/L012 nanoparticles and signal processing; (c) blue light was generated from Lipo@IR780/L012 nanoparticles under FUS irradiation (1.5 MHz, pulse 100 ms on, 900 ms off, 1 Hz, 6.2 MPa): (i) the photography of Lipo@IR780/L012 nanoparticles when the FUS was off and (ii) on; (iii) 470 nm blue light emission from Lipo@IR780/L012 nanoparticles under the repetitive FUS irradiation. (d) quantification analysis of light density indicating the light emission of Lipo@IR780/L012 nanoparticles increases linearly with FUS peak pressure; (e) Lipo@IR780/L012 nanoparticles exhibited high sensitivity to FUS irradiation frequencies, which had no influence on light emission (n=4 per group, one-way Analysis of Variance (ANOVA)); (1.5 MHz, 6.2 MPa); (f) Lipo@IR780/L012 nanoparticles exhibited high sensitivity to FUS, and pulse interval had no influence on light emission (n=4 per group, one-way ANOVA); (g) the blue light emission from Lipo@IR780/L012 nanoparticles upon FUS irradiation under 10 mm pork skin (1.5 MHz, pulse 100 ms on, 900 ms off, 1 Hz, 6.2 MPa); (h) quantification analysis of light density at different pork skin depth (1.5 MHz, pulse 100 ms on, 900 ms off, 1 Hz, 6.2 MPa) under FUS irradiation; (i) the light density of Lipo@IR780/L012 nanoparticles decayed with continuous FUS irradiation. All plots show mean±SEM unless otherwise mentioned. *P<0.05, **P<0.01, ***P<0.001, ****P<0.0001; ns, not significant.

FIGS. 4a-f. The in vitro sono-optogenetic stimulation and biosafety tests of Lipo@IR780/L012. (a) Illustration of FUS triggered CheRiff channels opening due to 470 nm blue light emission from Lipo@IR780/L012 nanoparticles. The flow of Ca²⁺ into the cells binds with jREGCO1a proteins to enhance the red fluorescence signal. (b) fluorescence images of CheRiff expressed spiking HEK cells with and without sonoluminescence irradiation from Lipo@IR780/L012 nanoparticles: (i) ultrasound off, (ii) ultrasound on; (c) fluorescence signal recording from CheRiff expressed spiking HEK cells under the following conditions: (i) no FUS, no Lipo@IR780/L012 nanoparticles; (ii) with FUS (1.5 MHz, puls 100 ms on, 900 ms off, 1 Hz, 6.2 MPa), no Lipo@IR780/L012 nanoparticles; (iii) no FUS, with Lipo@IR780/L012 nanoparticles; (iv) with FUS (1.5 MHz, puls 100 ms on, 900 ms off, 1 Hz, 6.2 MPa), with Lipo@IR780/L012 nanoparticles; (d) spike probability of CheRiff expressed spiking HEK cells under the different conditions (n=4 per group, one-way ANOVA). (e) cell viability tests of Lipo@IR780/L012 nanoparticles in HEK cells with and without FUS irradiation (n=5 per group); (f) hemolysis tests of Lipo@IR780/L012 nanoparticles (n=3 per group); All plots show mean±SEM unless otherwise mentioned. *P<0.05, **P<0.01, ***P<0.001, ****P<0.0001; ns, not significant

FIGS. 5a-f. The in vivo sono-optogenetic motor cortex stimulation. (a) Schematic of in vivo noninvasive sono-optogenetic brain stimulation, (i) the mouse was fixed in a stereotaxic frame and deeply anesthetized with 2.5% isoflurane, then Lipo@IR780/L012 nanoparticles were injected through tail vein. FUS transducer was in direct contact with the scalp of the mouse during brain stimulation, (ii) photograph of the in vivo sono-optogenetics, (iii) motor cortex zone was irradiated via FUS; (b) the limbs' response to FUS was recorded via camera, and analyzed via DeepLabCut, (i) photograph of limbs' response to FUS, different color dots were marked on joints to track the movement, (ii) kinematic joint angle changes of hip-knee (θ) and knee-feet (φ) response to FUS were tracked and calculated through DeepLabCut. (c) Time-resolved right limb's hip-knee response and (d) knee-feet response to FUS, (i) no FUS, no Lipo@IR780/L012 nanoparticles; (ii) with FUS (1.5 MHz, puls 100 ms on, 900 ms off, 1 Hz, 6.2 MPa), no Lipo@IR780/L012 nanoparticles; (iii) no FUS, with Lipo@IR780/L012 nanoparticles; (iv) with FUS (1.5 MHz, puls 100 ms on, 900 ms off, 1 Hz, 6.2 MPa), with Lipo@IR780/L012 nanoparticles; (e) Statistical analysis of the hip-knee and (f) knee-feet angle changes in different groups of subjects (n=4 per group, one-way ANOVA) in response to FUS irradiation. All plots show mean±SEM unless otherwise mentioned. *P<0.05, **P<0.01, ***P<0.001, ****P<0.0001; ns, not significant.

FIGS. 6a-d. Characterizations of various liposomes. (a) TEM image of blank liposome; (b) the concentration calibration curve of IR 780 in water; (c) the concentration calibration curve of L012 in water; (d) the zeta potential of Lipo@IR780/L012 nanoparticles determined via DLS.

FIGS. 7a-f. The ROS detection under the FUS irradiation. (a) time dependent UV-Vis degradation spectrum of DPBF indicating there was no ¹O₂ generation via Lipo@IR780 nanoparticles without FUS irradiation; (b) quantification analysis of DPBF decomposition in presence of Lipo@IR780 under the different FUS peak pressure; (c) time dependent UV-Vis degradation spectrum of SA indicating ·OH was not generated via Lipo@IR780 in absence of FUS (1.5 MHz, peak pressure 6.2 MPa); (d) the quantification analysis of SA decomposition in presence of Lipo@IR780 under the different FUS peak pressure; (e) time dependent UV-Vis degradation spectrum of SA indicating there were no OH residues when Lipo@IR780/L012 nanoparticles were treated via FUS (1.5 MHz, peak pressure 6.2 MPa); (f) quantification analysis of SA decomposition in presence of Lipo@IR780/L012 with/without FUS irradiation.

FIG. 8. The calibration curve of peak pressure.

FIGS. 9a-d. 470 nm blue light emission from Lipo@IR780/L012 nanoparticles under the FUS irradiation with different peak pressure (1.5 MHz, pulse 100 ms on, 900 ms off, 1 Hz). (a) 2.2 MPa; (b) 3.2 MPa; (c) 4.2 MPa; (d) 5.2 MPa.

FIGS. 10a-d. 470 nm blue light emission from Lipo@IR780/L012 nanoparticles under different frequency FUS pulse irradiation (1.5 MHz, 6.2 MPa). (a) 1 Hz (pulse 100 ms on, 900 ms off); (b) 2 Hz (pulse 100 ms on, 400 ms off); (c) 4 Hz (pulse 100 ms on, 150 ms off); (d) 8 Hz (pulse 50 ms on, 75 ms off).

FIGS. 11a-d. 470 nm blue light emission from Lipo@IR780/L012 nanoparticles under different pulse FUS irradiation (1.5 MHz, 6.2 MPa). (a) 100 ms (pulse 100 ms on, 1000 ms off); (b) 300 ms (pulse 300 ms on, 1000 ms off); (c) 500 ms (pulse 500 ms on, 1000 ms off); (d) 1000 ms (pulse 1000 ms on, 1000 ms off).

FIGS. 12a-d. 470 nm blue light emission from Lipo@IR780/L012 nanoparticles in different depth pork skin under different pulse FUS irradiation (1.5 MHz, 6.2 MPa, ultrasound pulse 100 ms on, 900 ms off). (a) there was no pork skin; (b) the pork skin depth was 3 mm; (c) the pork skin depth was 5 mm; (d) the pork skin depth was 15 mm.

FIGS. 13a-f. DeepLabCut framework in extracting kinematics data. (a) Images were segmented, down-sampled and extracted into batches of 20 images per video through the use of k-means algorithm; (b) Position of the hip, knee, and joints of the mice were determined by initially labeling 400 images manually for both lower extremities of the mice; (c) 380 of the labeled images (95%) was used for training the neural network model and 20 of the labeled images (5%) were used for verification and evaluation; (d) Training of the neural network model using ResNet50 was implemented for 500,000 epochs to ensure convergence of results. (e) The predicted pose was evaluated with the validation set to determine the maximum error in prediction of the (x,y) coordinates in terms of pixels; (f) Extracted (x,y) coordinates of the pose enables calculation of the segment orientation (joint angle) of interest yielding general movement. Differentiation of segment orientation with respect to time was followed to determine twitch movement as a result of ultrasound stimulation.

FIGS. 14a-b. Time-resolved left limb's hip-knee response. (a) and knee-feet response (b) to FUS in first mouse group, (i) no FUS, no Lipo@IR780/L012 nanoparticles; (ii) with FUS (1.5 MHz, puls 100 ms on, 900 ms off, 1 Hz, 6.2 MPa), no Lipo@IR780/L012 nanoparticles; (iii) no FUS, with Lipo@IR780/L012 nanoparticles; (iv) with FUS (1.5 MHz, puls 100 ms on, 900 ms off, 1 Hz, 6.2 MPa), with Lipo@IR780/L012 nanoparticles.

FIGS. 15a-h. FUS-activated nanotransducers act as a wireless light source for spatiotemporal neuromodulation. (a) Schematic of the neural activation through FUS triggered blue light emission from Lipo@IR780/L012/CaO₂ mechanoluminescent liposomes at focus; (b) Mechanism of FUS triggered light emission from self-amplifying mechanoluminescent nanoparticles. In this scheme, the ultrasound energy is absorbed through sonosensitizer IR780 to generate free radicals in the liposomes, and the ultrasound-induced mechanical force would also cause the perturbation of polyethylene glycol (PEG) 200 coating at the CaO₂ surface, thus enlarging the reaction with H₂O to generate H₂O₂ and to increase the pH in the lumen due to the generation of Ca(OH)₂. Accelerated free radicals and H₂O₂ production react with L012 to generate blue light, and the increased pH would improve the quantum yield of L012, thus achieving self-amplifying blue light emission; (c) XRD analysis of PEG 200 coated CaO₂ nanoparticles; (d) TEM images of PEG 200 coated CaO₂ nanoparticles; (e) XRD analysis of PEG 200 coated CaO₂ nanoparticles after FUS stimulation; (f) TEM image of Lipo@IR780/L012/CaO₂ liposomes; (g) Dynamic light scattering (DLS) tests of blank and payload liposomes in solution; (h) stability evaluation of payload liposomes in serum mimic solution tested by DLS.

FIGS. 16a-f. FUS triggered free radical generation and consumption by L012. (a) UV-Vis spectra of DPBF under the ultrasound irradiation (1.5 MHz, 1.55 MPa) over time, indicating the efficient generation of ¹O₂; (b) The quantitative analysis of DPBF decomposition with or without ultrasound irradiation (n>3 per group) in different nanoparticle solutions; (c) UV-Vis spectra of SA under the ultrasound irradiation (1.5 MHz, 1.55 MPa) over time; (d) The quantification analysis of SA decomposition with or without ultrasound irradiation (n>3 per group) in different nanoparticle solutions; The quantification analysis of (e) DPBF decomposition and (f) SA decomposition at the similar irradiation conditions after loading L012 over time, these results showed an absence of free radical residues in Lipo@IR780/L012/CaO₂ liposomes under the FUS irradiation.

FIGS. 17a-l. FUS triggered blue light emission and neuronal activation. (a) Schematic of the blue light emission from mechanoluminescence solution under the ultrasound irradiation; (b) photons were generated from Lipo@IR780/L012/CaO₂ liposomes under repetitive FUS irradiation (1.5 MHz, 1.55 MPa, pulse 50 ms on, 950 ms off); (c The latency time between ultrasound excitation and photon emission from Lipo@IR780/L012/CaO₂ liposomes at different ultrasound irradiation frequencies; (d) quantification analysis of light intensity from Lipo@IR780/L012 and Lipo@IR780/L012/CaO₂ liposomes under the similar ultrasound irradiation (1.5 MHz, 1.55 MPa, pulse 50 ms on, 950 ms off); (e) normalized ultrasound energy transmission efficiency in pork skin (1.5 MHz, 1.55 MPa); (f) quantification analysis of light intensity from Lipo@IR780/L012 and Lipo@IR780/L012/CaO₂ liposomes under the similar ultrasound irradiation (1.5 MHz, 1.55 MPa, pulse 50 ms on, 950 ms off) at different tissue depths; (g) Mechanoluminescence spectra of Lipo@IR780/L012 and Lipo@IR780/L012/CaO₂ liposomes, where the emission spectrum of the lipsomes is overlaid with the ChR2 opsin absorption spectrum (green dot curve); (h) Illustration of a ChR2 expressing neuron under ultrasound irradiation activating in presence of Lipo@IR780/L012/CaO₂ nanoparticles. The ChR2 opsin channel could be activated under blue light emission. The Ca2+imaging with JRGECO1a could be used to image the neuronal activation; (i) Fluorescent images of primary neurons expressing hSyn::ChR2-EYFP and hSyn::JRGECO1a-mCherry, scale bar: 20 μm; (j) JRGECO1a fluorescence signal recording of ChR2 expressing neurons in different experimental conditions, (i) FUS −, LipoCaO₂ −; (ii) FUS +, LipoCaO₂ −; (iii) FUS −, LipoCaO₂ +; (iv) FUS +, LipoCaO₂ +, FUS stimulation (1.5 MHz, 1.55 MPa, pulse 100 ms on 900 ms off); (k) Statistical analysis of JRGECO1a signal changes in different groups (n=3 per group, two-way ANOVA and multiple comparisons test); (l) Spike probability of ChR2 expressing primary neurons under the different conditions (n=3 per group, two-way ANOVA and multiple comparisons test). All plots show mean±SEM unless otherwise mentioned. *P<0.05, **P<0.01, ***P<0.001, ****P<0.0001; ns, not significant.

FIGS. 18a-g. In vivo sono-optogenetics for spatiotemporal motor cortex modulation. (a) Schematic of the remote motor cortex activation of sono-optogenetics for controlled limb motion. Lipo@IR780/L012/CaO₂ liposomes were injected into the M2 area in the right hemisphere. After 24 h, a FUS transducer with a focus on the motor cortex area was used to treat the mouse, the limb motion was recorded via camera and analyzed with DeeplabCut; (b) The blue light emission from mechanoluminescent liposomes under the FUS irradiation (1.5 MHz, 1.55 MPa, pulse 100 ms on 900 ms off); (c) Time-resolved left limb's motion and (d) right limb's motion in different experimental conditions, FUS −, LipoCaO₂−; FUS+, LipoCaO₂ −; FUS−, LipoCaO₂+ and FUS+, LipoCaO₂ +; (e) Statistical analysis of the right and left limbs' motions in different groups of subjects (n=5 per group, two-way ANOVA and multiple comparisons test) in response to FUS irradiation. (f) Confocal images of the right motor cortex region under different experimental conditions. Increased c-Fos signals triggered by FUS were only observed in the presence of both ChR2 opsins and mechanoluminescent liposomes, scale bar: 20 μm. (g) Statistical analysis of c-Fos signal densities under different experimental conditions at the M2 motor cortex region (n=4 per group, two-way ANOVA, and multiple comparisons test). All plots show mean±SEM unless otherwise mentioned. *P<0.05, **P<0.01, ***P<0.001, ****P<0.0001; ns, not significant.

FIGS. 19a-i. In vivo sono-optogenetics for spatiotemporal mouse VTA modulation. (a) Schematic of the remote VTA neuron activation of sono-optogenetics for lever press tests. Once the mouse presses the lever trigger, a FUS pulse is given (1.5 MHz, 1.55 MPa, pulse 100 ms); (b) The blue light emission from mechanoluminescence liposomes under the FUS irradiation (1.5 MHz, 1.55 MPa, pulse 100 ms on 900 ms off); (c) The mouse-lever-press curve at the pre-stimulus session, where the FUS generator was off to obtain the lever press baseline, (d) during FUS stimulation (or no FUS stimulation) epoch, where FUS is triggered on via the action of the mouse and (e) at post-stimulus epoch (FUS generator is off) under the different experimental conditions. (n=4 per group, 1.5 MHz, 1.55 MPa, pulse 100 ms); (f) Time courses of the total lever presses in each epoch for the mouse under the different experimental conditions (n=4 per group; 1.5 MHz, 1.55 MPa, pulse 100 ms; two-way ANOVA and multiple comparisons test); (g) Statistical analysis of mouse lever presses at all epochs (n=4 per group; two-way ANOVA and multiple comparisons test); (h) Confocal images of the VTA region under the different experimental conditions. Remarkable c-Fos signals triggered by FUS were only observed in the presence of both ChR2 opsins and mechanoluminescent liposomes, scale bar: 20 μm. (i) Statistical analysis of c-Fos signal densities under the different experimental conditions at the VTA region (n=4 per group, two-way ANOVA, and multiple comparisons test). All plots show mean±SEM unless otherwise mentioned. *P<0.05, **P<0.01, ***P<0.001, ****P<0.0001; ns, not significant.

FIGS. 20a-b. The fluorescence spectra of L012 (a) at different pH and (b) at different H2O2 concentrations.

FIG. 21. The XRD spectrum of CaO2 stored at solution for 24 h after loading into the LNP.

FIGS. 22a-f. (a) Time dependent UV-vis spectra of DPBF in Lipo@IR780/CaO2 liposomes without ultrasound irradiation at different time points, indicating no any generation of ¹O₂; (c) Time dependent UV-Vis spectra of SA in Lipo@IR780/CaO2 liposomes without the ultrasound irradiation at different points; (c) The quantification analysis of DPBF decomposition Lipo@IR780/CaO2 liposomes with ultrasound irradiation (n>3 per group) at different FUS peak pressure; (d) The quantification analysis of SA decomposition with ultrasound irradiation (n>3 per group) at different FUS peak pressure; (e) Time dependent UV-vis spectra of DPBF in Lipo@IR780/L012/CaO2 liposomes with ultrasound irradiation at different time points; (f) Time dependent UV-Vis spectra of SA in Lipo@IR780/L012/CaO2 liposomes without the ultrasound irradiation at different points.

FIGS. 23a-e. 470 nm blue light emission from Lipo@IR780/L012/CaO2 liposomes under different frequency FUS pulse irradiation (1.5 MHz, 1.55 MPa). (a) 1 Hz (pulse 100 ms on, 900 ms off); (b) 2 Hz (pulse 100 ms on, 400 ms off); (c) 4 Hz (pulse 100 ms on, 150 ms off); (d) 5 Hz (pulse 100 ms on, 100 ms off); (d) 10 Hz (pulse 50 ms on, 50 ms off).

FIGS. 24a-d. 470 nm blue light emission from Lipo@IR780/L012/CaO2 liposomes under the FUS irradiation with different peak pressure (1.5 MHz, pulse 100 ms on, 900 ms off, 1 Hz). (a) 0.51 MPa; (b) 0.89 MPa; (c) 1.08 MPa; (d) 1.40 MPa.

FIGS. 25a-c. 470 nm blue light emission from Lipo@IR780/L012/CaO2 liposomes in different depth pork skin under different pulse FUS irradiation (1.5 MHz, 1.5 MPa, pulse 100 ms on, 900 ms off). (a) there was no pork skin; (b) the pork skin depth was 3 mm; (c) the pork skin depth was 5 mm; (d) the pork skin depth was 8 mm; (e) the pork skin depth was 10 mm.

FIG. 26. The heatmap of FUS peak pressure at mouse motor cortex region, where 1.55 MPa primary FUS peak pressure was used (1.5 MHz, pulse 100 ms on 900 ms off).

FIG. 27. Confocal images of the left motor cortex region under the different experimental conditions, FUS −, LipoCaO₂ −; FUS +, LipoCaO₂ −; FUS −, LipoCaO₂ + and FUS +, LipoCaO₂ +; Scale bar: 20 μm.

FIGS. 28a-c. The photographs of lever press testing systems, including (a) trigger and holder; (b) Microcontroller Unit (MCU) system, and (c) FUS generator.

FIG. 29. The heatmap of FUS peak pressure at the mouse VTA region, where 1.55 MPa primary FUS peak pressure was used (1.5 MHz, pulse 100 ms on 900 ms off).

FIG. 30. H&E staining of brains treated at different conditions after 7 days (Saline and Lipo@IR780/L012/CaO2 liposomes with FUS stimulation), Scale bar: 100 μm.

FIGS. 31a-b. The biosafety evaluation through microglia activation marker Iba1 immunostaining. (a) The Iba1 fluorescence images of mouse brain after treatment at different conditions after 7 days; (b) Statical analysis of Iba1 intensity.

FIGS. 32a-b. The biosafety evaluation through neuron apoptosis marker Caspase-3 immunostaining. (a) The Caspase-3 fluorescence images of mouse brain after treatment at different conditions after 7 days; (b) Statical analysis of Caspase-3 intensity.

### DESCRIPTION OF ILLUSTRATIVE EMBODIMENTS

Organic nanoparticles and liposomes are provided herein that can be used for generating light in the brain of a mammalian subject (e.g., for use in optogentic methods).

Biocompatible mechanoluminescence systems are provided that include organic lipid vehicles (e.g., liposomes), sonosensitizer (e.g., IR780), and a chemiluminescence compound (e.g., L012) to achieve noninvasive optogenetic stimulation of neural activity under focused ultrasound, without the need for charging of the nanoparticles. In some embodiments, ultrasound energy is noninvasively transmitted to deep brain tissue and sensed via the sonosensitizer (e.g., IR780), generating ROS that triggers the chemiluminescence compound (e.g., L012), resulting in blue light emission. In some embodiments, the emitted light can be detected in methods for imaging a tissue. Data is provided herein showing that the liposomes and nanoparticles can be used to generate light for stimulation of receptors (e.g., opsins) in optogenetic methods in vivo.

Self-amplifying liposomal nanotransducers triggered by FUS to emit light for deep brain sono-optogenetics are also provided herein. Ultrasound energy can be noninvasively delivered to the deep brain via pressure waves with high transmission efficiency and subsequently sensed by a sonosensitizer (e.g., IR780) and a sono-amplifier (e.g., CaO₂) to produce spatiotemporal blue light via a controlled cascade reaction in liposomes. In vitro and in vivo results support the idea that ChR2-expressing neurons can be spatiotemporally controlled via irradiation by sono-mechanoluminescence, thus achieving temporal neuron activation at deeper regions in the brain (e.g., the motor cortex and VTA in mice) for behavioral modulation. Excellent biosafety and biocompatibility data were observed and support the use of sono-optogenetic systems provided herein for minimally invasive, targeted deep brain modulation for large animals or clinical applications.

### I. DEFINITIONS

The terms “applying” and “administering” are used interchangeably to refer to causing a subject to receive a treatment. For example, applying an ultrasound signal means generating an ultrasound signal and directing it into a region of the subject's body. As another example, administering a particle to the subject means moving the particle inside the body of the subject.

The terms “active agent,” “active pharmaceutical ingredient,” “pharmacologically active agent,” and “drug” are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “treatment,” “treating,” and the like, refer to obtaining a desired pharmacologic and/or physiologic effect, such as reduction of the effects of a neurological disease. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having; (b) inhibiting progression of a disease; and (c) reducing a symptom of the disease or causing regression of the disease (e.g., reduction in neurological effects or functioning of a neurological disease).

A “therapeutically effective amount”, a “therapeutically effective dose” or “therapeutic dose” is an amount sufficient to effect desired clinical results (e.g., achieve therapeutic efficacy, achieve a desired therapeutic response). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., neurodegenerative disease, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including intravenous, buccal, rectal, parenteral, intraperitoneal, intradermal, intracheal, intramuscular, subcutaneous, and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “sonosensitizer” refers to a compound that can generate a free radical such as a reactive oxygen species (ROS) in response to the application of ultrasound stimulation (e.g., focused ultrasound or FUS). Preferably, the sonosensitizer generates ROS in response to physical stimulation by ultrasound stimulation such as FUS. It is anticipated that a wide variety of chemical sonosensitizers can be used in embodiments of the present disclosure.

The term “chemiluminescent” means that a nanoparticle or liposome can luminesce in response to a compound (e.g., a reactive oxygen species) produced by a sonosensitizer. The chemiluminescent emission of light can include the emission of a single photon of light, or of two or more photons of light, such as 5 photons or more or 100 photons or more. The chemiluminescent emission of light can also include the emission of light of an intensity that is detectable by a light detector.

The term “sono-amplifier” refers to a compound that can generate a free radical such as preferably a singlet oxygen (¹O₂) and/or a hydroxyl radical (·OH). In some preferred embodiments, the sono-amplifier is a peroxide such as, e.g., calcium peroxide (CaO₂).

### I. NANOPARTICLES AND LIPOSOMES

In some aspects, the present disclosure provides organic nanoparticles that can be used to generate light in the brain based on stimulation using ultrasound. Organic nanoparticle light sources using liposomes for non-invasive deep brain optogenetic stimulation under FUS. In contrast to previous inorganic nanoparticles, organic nanoparticles and liposomes are provided (e.g., liposomes containing IR780 and L012) that display biocompatibility and minimum toxicity to the cells even at higher concentrations (e.g., even at up to 200 μg/mL).

In some aspects, nanoparticles and liposomes that comprise a sonosensitizer and a chemiluminescent compound are provided. Preferably, due to the application of ultrasound the sonosensitizer can generate a reaction product such as a free radical or reactive oxygen species that can stimulate the chemiluminescent compound to generate light. The liposomes may be unilamellar liposomes, multilamellar liposomes, or multivesicular liposomes. In some embodiments, the liposomes are unilamellar liposomes that may, e.g., be produced via film hydration methods.

Attributing to its natural constituents, liposomes are effectively metabolized in the body. Liposomes were the first nanodrugs in FDA clinical trials and have been extensively applied in nanomedicines since the first liposomal formulation was approved by FDA in mid-1990s (Bobo et. al., 2016; Tyrrell et. al., 1976; Allen et. al., 19950. More recently, the liposome based mRNA vaccine developed by BioNTech/Pfizer and Moderna was clinically applied against COVID-19 (Schoenmaker et. al., 2021).

The liposomes may comprise a variety of lipid components. In some preferred embodiments, the liposomes contain one or more an aqueous internal chambers. The liposomes are preferably biodegradable. The liposomes may comprise phospholipid, 1,2-distearoryl-sn-glycero-3-phosphatidyl choline (DSPC), sphingomyelin, phosphatidylcholine (e.g., egg phosphatidylcholine or soy phosphatidylcholine), monosialoganglioside, cholesterol, polyethylene glycol (PEG), PEG-succinyl cysteine (PEG-SC), poly (lactic-co-glycolic acid) (PLGA), dioleoylphosphatidylethanolamine (DOPE), 1,2-dipalmitoyl-sn-glycero-3-phosphocholine (DPPC), cholesterol, 1,2-distearoyl-sn-glycero-3-phosphoethanolamine-N-[amino(polyethylene glycol)] (DSPE-PEG), choline phosphate. In some embodiments, the liposomes may comprise polyethylene glycol (PEG) or be stealth liposomes. In some embodiments, the following liposomes can be used for drug delivery, bioimaging, light induced catalyst, etc.

The liposomes or nanoparticles may be a variety of sizes. For example, in some embodiments, the liposomes are about 10-1000 nm, 20-750 nm, 50-500 nm, 25-250 nm, 50-300 nm, 25, 50, 75, 100, 125, 150, 175, 200, 250, 275, 300, 350, 400, 500, 600, 750 nm, or any ranger derivable therein. In some preferred embodiments, the liposomes or nanoparticles are 100-200 nm.

The liposomes may comprise three primary constituents: a chemiluminescent compound (e.g., L012), a sonosensitizer (e.g., IR-780) and a lipid vehicle. In some embodiments, L012 and IR-780 are loaded into the lipids to act as nanoscopic light sources under the FUS irradiation. Specifically, the sonosensitizer IR-780 generates free radicals by transferring ultrasound energy to nearby oxygen or water molecules through acoustic cavitation (Choi et. al., 2020; Zhang et. al., 2019). The chemiluminescent L012 is then activated by free radicals, producing light which may result in activating opsins expressed by specific types of neurons for controlling animal behaviors or treating diseases (Scheme 1).

The liposomes or nanoparticles comprise both a sonosensitizer and a chemiluminescent compound. Stimulation with ultrasound can cause the sonosensitizer to stimulate the chemiluminescent compound to generate light. In some embodiments the liposomes or nanoparticles contain both the sonosensitizer and the chemiluminescent compound. Nonetheless, it is anticipated that in some embodiments

The organic nanoparticles or liposomes can be administered to a subject via a variety of administration routes. For example, the nanoparticles or liposomes can be administered parenterally, intravenously, intracerebrally, subcutaneously, or intranasally.

A variety of dosages of nanoparticles or liposomes may be administered to a mammalian subject. For example, the nanoparticles or liposomes may be administered to the subject in an amount of about 5-10 mg/kg, 0.5, 1, 2, 3, 4, 5, 6, 7, 8, 9, 10, 12, or 15 mg/kg, or any range derivable therein.

### A. Sonosensitizers

A variety of sonosensitizers may be included in organic nanoparticles or liposomes provided herein. The sonosensitizer may generate free radicals such as reactive oxygen species (ROS) or hydroxide radical. As shown in the below examples, it has been observed that stimulation of a sonosensitizer in a nanoparticle or liposome with ultrasound stimulation (e.g., FUS) can be used to cause a chemiluminescent compound inside of or in the vicinity of the nanoparticle or liposome to emit light, without causing significant toxicity or cell death to the nearby tissues.

Several classes of sonosensitizers exist that can be used. For example, the sonosensitizer may be an organic sonosensitizer such porphyrins, phthalocyanines, xanthenes, indocyanines, natural products, and quinolones etc. (e.g., Xing et al., Coordination Chemistry Reviews 445(15), 2021). The core of a porphyrin is a tetrapyrrole in which the four rings of the pyrrole type are linked together by methine carbon atoms. Phthalocyanine is an intensely blue-green-colored aromatic macrocyclic compound that is widely used in dyeing and the first phthalocyanine (Pc) was synthesized accidentally in 1907 as an unidentified blue compound when o-cyanobenzamide was heated at high temperature and that substance is currently known to be the metal-free phthalocyanine. Xanthene derivatives are a class of heterocyclic compounds that have been observed in some bioactive natural products. Select sonosensitizers are shown below in Table 1.

TABLE 1
Example Sonosensitizers

| | |
|---|---|
| A | 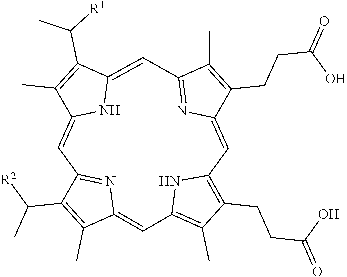 |
| | HMME: R¹,R² = OCH [symbol] •OH or OH [symbol] OCH₃ |
| | 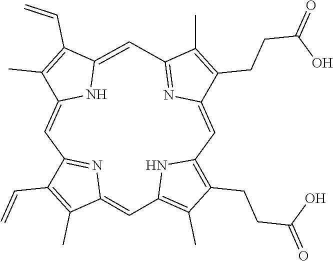 [SMILES: C=CC1=C(C)c2cc3[nH]c(cc4nc(cc5[nH]c(cc1n2)c(C)c5C=C)C(C)=C4CCC(=O)O)c(CCC(=O)O)c3C] |
| | Protoporphyrin (PpIX) |
| | 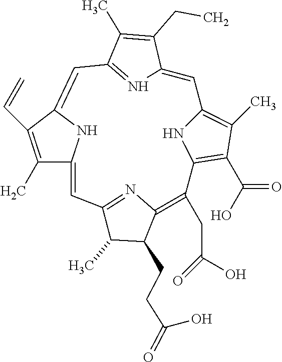 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C] |
| | Chlorin e6 (Ce6) |
| B | 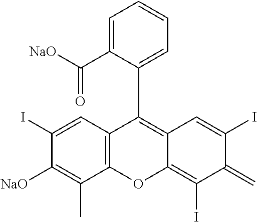 |
| | Erythosin B (EB) |
| | 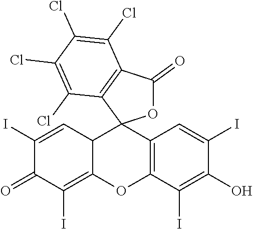 [SMILES: O=C1C(I)=CC2C(=C1I)Oc1c(cc(I)c(O)c1I)C21OC(=O)c2c(Cl)c(Cl)c(Cl)c(Cl)c21] |
| | Rose bengal (RB) |
| | 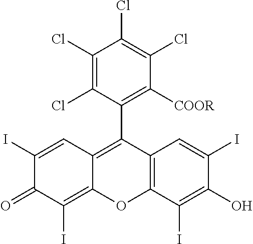 |
| | RBD2 R = (CH₂)₇COOH |
| | RBD3 R = CHCOOH(CH [symbol] ) [symbol] CH [symbol] |
| C | 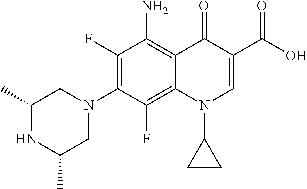 [SMILES: C[C@@H]1CN(c2c(F)c(N)c3c(=O)c(C(=O)O)cn(C4CC4)c3c2F)C[C@H](C)N1] |
| | Sparfloxacin (SPFX) |
| | 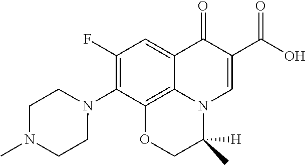 [SMILES: C[C@H]1COc2c(N3CCN(C)CC3)c(F)cc3c(=O)c(C(=O)O)cn1c23] |
| | Levofloxacin (LVFX) |
| | 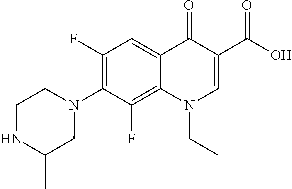 [SMILES: CCn1cc(C(=O)O)c(=O)c2cc(F)c(N3CCNC(C)C3)c(F)c21] |
| | Lomefloxacin (LFLX) |
| D | 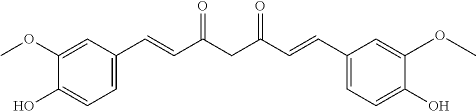 |
| | Curcumin |
| | 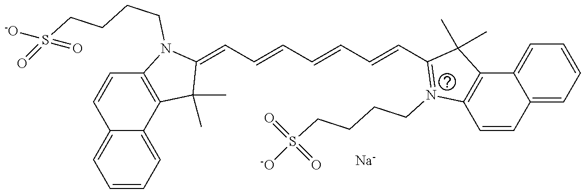 |
| | Indocyanine green (ICG) |
| | 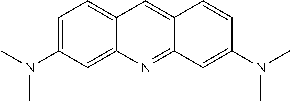 |
| | Acridine orange (AO) |
| | 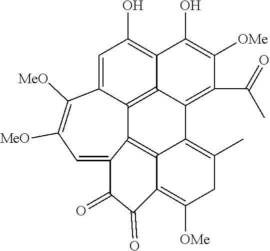 [SMILES: COC1=C(OC)c2cc(O)c3c(O)c(OC)c(C(C)=O)c4c5c6c(c(=O)c(=O)c(c6c2c34)=C1)=C(OC)CC=5C] |
| | Hypocrellin B |
| | 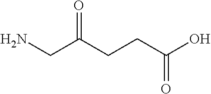 |
| | 5-aminolevulinic acid (5-ALA) |

[symbol]

indicates data missing or illegible when filed

Chemical structures of porphyrin-based sonosensitizers (A), xanthene-based sonosensitizers (B), non-steroidal anti-inflammatory drug-based sonosensitizers (C), and other sonosensitizers (D) are provided above. In some embodiments, the sonosensitizer is DCPH-P-Na (I), hematoporphyrin, zinc protoporphyrin, methylene blue, TiO₂, or Chlorin e6.

Sonosensitizers that can be included in a nanoparticle (e.g., lipid nanoparticle) or liposomes of the present disclosure includes, e.g., IR-780, DCPH-P-Na (I), hematoporphyrin, zinc protoporphyrin, methylene blue, TiO2, and/or Chlorin e6. Although in some embodiments a liposome or nanoparticle (e.g., lipid nanoparticle) contains a single type of sonosensitizer, in some embodiments the liposome or nanoparticle contains two or more types of sonosensitizers. The sonosensitizer preferably generates free radicals in response to ultrasound administration.

Sonosensitizers have previously been used in cancer therapies to kill cells due to the generation of ROS near cancerous cells. Sonodynamic therapy (SDT), which involves a combination of low-intensity ultrasound (US) and a chemical sonosensitizer, has emerged as a promising minimally invasive and selective approach for the treatment of deep tumors. US not only increases cell membrane permeability, thereby enhancing the cellular uptake efficiency of chemical sonosensitizers, but also excites chemical sonosensitizers in deep tissues to generate reactive oxygen species (ROS), which in turn kill cancer cells.

In some embodiments the sonosensitizer is IR780. The sonosensitizer IR780 has been reported to generate ROS under FUS stimulation due to acoustic cavitation and has been extensively developed for cancer sonodynamic therapy (Choi et. al., 2020; Zhang et. al., 2019; Baker et. al., 2001; Meng et. al., 2021). Singlet oxygen (¹O₂) and hydroxyl radical (·OH) are the main species of ROS generated in sonochemistry (Chang et. al., 2019).

### B. Sono-Amplifiers

Liposomes and nanoparticles provided herein may in come preferred embodiments include a sono-amplifier such as a peroxide. Self-amplifying sono-mechanoluminescence nanotransducers may utilize a sono-amplifier from within the liposomes to generate additional free radicals and hence increase the sensitivity and response to mechanoluminescent stimulation. Synchronized blue light with low responsive latency can be generated under the FUS stimulation through a self-amplifying cascade reaction in these liposomes. This sono-optogenetic system can be used to achieve deep brain photon delivery to brain tissues (e.g., the motor cortex and/or ventral tegmental area in mice), and these composition can be used to activate ChR2-expressing neurons for remote and minimally invasive behavioral control. Preferably, the sono-amplifier can generate free radicals such as oxygen free radicals when the surface of the liposome is either perturbed or disrupted by mechanical or sound stimulation or when a sonosensitizer in the nanoparticle or liposome generates free radicals.

The sono-amplifier may be a peroxide. In some preferred embodiments, the peroxide is calcium peroxide (CaO₂). The calcium peroxide may be present in the liposomes in an amount of about 1-10 (wt.) %, or about 1, 2, 3, 4, 5, 6, 7, 8, 9, 10 (wt.) % or any range derivable therein. The sono-amplifier may be present in the liposomes in an amount of about 1-10 (wt.) %, or about 1, 2, 3, 4, 5, 6, 7, 8, 9, 10 (wt.) % or any range derivable therein. (wt.) % refers to percent by weight (also called weight/weight percent).

C. Polyethylene glycol (PEG)

Liposomes and nanoparticles may contain a polyethylene glycol (PEG) on the surface or membrane of the liposomes or nanoparticle. For example, the PEG may beneficially provide additional membrane integrity for liposomes or nanoparticles that contain a sono-amplifier, thus reducing the chances that the sono-amplifier will generate free radicals prior to mechanical or luminescent stimulation of the liposomes or nanoparticles. It is also anticipated that including PEG on the surface of the liposome or nanoparticle may also favorably affect biocompatibility and/or pharmacokinetics of the liposomes or nanoparticles.

A variety of PEG can be included in the liposomes or nanoparticles. For example, in some preferred embodiments, the PEG has a molecular weight of 100-10,000, more preferably 100-5000, even more preferably 100-1000, 100-500, 100, 150, 200, 250, 300, 360, 400, 450, 500 Daltons (Da), or any range derivable therein (e.g., PEG-100, PEG-150, PEG-200, PEG-250, PEG-300, PEG-360, PEG-400, PEG-450, PEG-500). For example, in some embodiments the PEG is PEG-200. The PEG (e.g., PEG100-400, PEG200, etc.) may be present in an amount of about 5-20 (wt.) %, or about 5, 6, 7, 8, 9, 10, 11,12, 13, 14, 15, 16, 17, 18, 19, 20, 21, 22, 23, 24, 25 (wt.) % or any range derivable therein. (wt.) % refers to percent by weight (also called weight/weight percent). It is further anticipated that other biocompatible polymers can be included in the liposomes or nanoparticles, such as, e.g., glycerol. In some embodiments the nanoparticles are PEGylated liposomes or “stealth” liposomes. It is anticipated that including a PEG-containing lipids with molar weights of 1900-5000 Da may prolong circulation time in a mammalian subject. The PEGylation of the liposome can be used to achieve various coverage of the surface of the liposome; for example: <5 mol. % PEG may result in less than 100% coverage of the liposome, about 5-15 mol. % PEG may result in about 100% coverage of the liposome (in a mushroom-or brush-like shape), and greater than about 15% PEG may result in about 100% coverage of the liposome (in a brush-like shape). In some embodiments, the PEG is included in the liposome in an amount of about 0.1-5 mol. %, or about 0.1, 0.25, 0.5, 0.75, 1, 1.5, 2, 2.5, 3, 3.5, 4, 4.5, 5 mol. %, or any range derivable therein. mol. % refers to mole percent.

PEGylation of liposomes or nanoparticles can be performed via several methods known in the art, including addition of PEG-lipids to lipid composition before liposome formation (pre-insertion method) or mixing of PEG-lipids and liposomal dispersion (post-insertion method) e.g., as described in Nosova et al. (2019). Both the length and coverage density of PEG influence the liposomal PEGylation efficiency. Very short PEG molecules cannot prevent protein absorption and increase the blood circulation time, while very long PEG chains lead to significant decline in transfection activity.

### D. Chemiluminescent Compounds

Chemiluminescent compounds (also referred to as chemiluminescence compounds) as used herein refers to compounds that can generate light in response to free radicals (e.g., ROS) that can be produced by a sonosensitizer. In some preferred embodiments, a nanoparticle (e.g., a lipid nanoparticle) or a liposome of the present disclosure comprises a chemiluminescent compound that produces light in response to a singlet oxygen (¹O₂) or hydroxyl radical (·OH).

It is anticipated that a variety of chemiluminescent compounds can be used. For example, chemiluminescent compounds that may be used include L012, dioxetane derivatives, luminol, isoluminol, imidazopyrazinone derivatives, lophine derivatives and acridinium derivatives. In some embodiments, the chemiluminescent compound is L012.

The chemiluminescent compound may emit a range of wavelengths of light in response to stimulation by a free radical or ROS. For example, the chemiluminescent compound may emit a blue wavelength of about 430-550 nm. The wavelength of the light emitted may range from 250 nm to 650 nm, such as from 300 nm to 550 nm or from 350 nm to 450 nm. In some cases, the emitted photon has a wavelength ranging from 350 nm to 2000 nm, such as from 400 nm to 1700 nm, from 400 nm to 1300 nm, from 400 nm to 1000 nm, from 400 nm 800 nm, from 400 nm to 650 nm, or from 450 nm to 500 nm. In some cases, 80% or more of the emitted photons have such wavelengths, such as 90% or more or 95% or more. As used herein, the term “Stokes shift” refers to the difference between the absorption and emission wavelengths, and the Stokes shift can range in some embodiments from 20 nm to 100 nm, such as from 60 nm to 80 nm. In some embodiments, 80% or more of the wavelength of light emitted by the nanoparticles or liposomes is from about 400-700 nm, 400, 450, 500, 550, 600, 650, 700 nm, or any range derivable therein.

The light emitted by the chemiluminescent compound can be utilized in a variety of methods. Contacting a tissue inside a subject with light can be used in applications including optogenetics, photodynamic therapy, selective gene editing, and fluorescent imaging. In optogenetics, a neuron that includes a photosensitive protein can be contacted with light, thereby modulating the neuron. Thus, optogenetics allows for the study of neural circuits by specifically activating or deactivating particular neurons.

### II. ULTRASOUND

Ultrasound may be applied to a nanoparticle or liposomes of the present disclosure that contains a sonosensitizer and a chemiluminescent compound, in order to stimulate the sonosensitizer to generate a free radical such as ROS, which then stimulates the chemiluminescent compound to emit light. The terms “ultrasound signal” and “ultrasound” are used interchangeably to refer to sound waves with frequencies that are above what a human can hear, e.g., above 20 kHz. Sound waves are physical vibrations that propagate through a medium, e.g., through air, water, bone, and muscle. In some cases the ultrasound has a frequency ranging from 20 kHz to 100 MHz, such as from 100 kHz to 15 MHz, or from 500 kHz to 5 MHz. The ultrasound signal can be a focused ultrasound signal (FUS) or an unfocused ultrasound signal. In focused ultrasound, the ultrasound emitting device is typically configured such that the highest intensity ultrasound signal is located not only at a particular angle relative to the device, but also at a particular distance away from the ultrasound emitter part of the ultrasound emitting device. For instance, the highest intensity ultrasound signal can be located between 10 mm and 150 mm away from the end of the ultrasound emitter, e.g., 10 mm to 150 mm below the skin, such as between 30 mm and 75 mm. A medical professional can change the settings on the ultrasound emitting device, thereby changing the desired depth of highest ultrasound intensity. This can allow for lower intensity ultrasound applied to a more superficial location, such as the prefrontal cortex of the brain, while also providing for high intensity ultrasound at an interior location, such as Broca' s area of the brain, which is located underneath the prefrontal cortex. In contrast, in unfocused ultrasound the highest intensity is at or near the surface of the skin or tissue, with ultrasound intensity decreasing with increasing distance from the emitter. One of skill can modify the ultrasound or FUS signal to stimulate particular tissues within a subject such as a mammalian subject. In some embodiments, the ultrasound or FUS is applied to the brain, spinal cord, peripheral nerves, or other tissue in the subject.

The ultrasound is applied to a nanoparticle or liposome in proximity to the tissue of the subject. As used herein, “in proximity to” means that at least one of the photons emitted by the nanoparticle or liposome will contact the tissue. For example, the tissue can be a group of neuron cells in the auditory complex of the brain, and the particle can be located inside a blood vessel adjacent to the auditory complex. A photon emitted by the particle may pass through the liquid of the blood vessel, pass through the wall of the blood vessel, and then enter a neuron of the auditory complex. In such an embodiment, the photoexcited mechanoluminescent particle was in proximity to the auditory complex tissue since the emitted photon contacted a cell of the target tissue. As described above, photons can be absorbed by body tissue, wherein the degree of absorption depends on the wavelength of light, the type of tissue, and the distance of tissue the photon is passing through. As such, the distance referred to by the term “in proximity to” depends on certain aspects of the method, and is not limited to a particular range. However, in some cases the particle is 10 mm or less from the tissue, such as 1 mm or less, 100 pm or less, or 10 pm or less. The term “in proximity to” also includes cases wherein the particle is within the tissue that is to be contacted with light. For example, the particle can be within a blood vessel inside the auditory complex, or it can be within a blood vessel inside a cancerous tumor of the liver.

It is anticipated that a nanoparticle or liposome of the present disclosure may emit different wavelengths of light depending on the particular chemiluminescent compound that is stimulated by or activated by ultrasound stimulation of the sonosensitizer. The photoexcitation photon may for example have a blue wavelength of about 430-550 nm. The wavelength of the light emitted may range from 250 nm to 650 nm, such as from 300 nm to 550 nm or from 350 nm to 450 nm. In some cases, the emitted photon has a wavelength ranging from 350 nm to 2000 nm, such as from 400 nm to 1700 nm, from 400 nm to 1300 nm, from 400 nm to 1000 nm, from 400 nm 800 nm, from 400 nm to 650 nm, or from 450 nm to 500 nm. In some cases, 80% or more of the emitted photons have such wavelengths, such as 90% or more or 95% or more. As used herein, the term “Stokes shift” refers to the difference between the absorption and emission wavelengths, and the Stokes shift can range in some embodiments from 20 nm to 100 nm, such as from 60 nm to 80 nm. In some embodiments, 80% or more of the wavelength of light emitted by the nanoparticles or liposomes is from about 400-700 nm, 400, 450, 500, 550, 600, 650, 700 nm, or any range derivable therein.

The term “tissue” refers to any region of the subject's body. For example, the tissue can be a brain tissue, e.g. the auditory complex, the prefrontal cortex, or the hippocampus, a muscle tissue, a region of bone, and a liver tissue. In cases wherein the tissue is a brain tissue, the term “brain tissue” includes neurons, glia cells, extracellular fluid, and other components of the brain. In some cases the tissue is a cancerous tumor of any part of the body.

The amount of time between administering the nanoparticles or liposomes to the subject and the application of light can be any suitable length. In some cases, such a time is 180 minutes or less, 120 minutes or less, 60 minutes or less, 30 minutes or less, 10 minutes or less, of 5 minutes or less.

An ultrasound can be applied for the desired amount of time, for example from ranges from about 1 second to about 30 minutes, such as from 1 second to 10 minutes, or from 1 second to 1 minute. The nanoparticles or liposomes may disperse in the blood of body of the subject after administration, so it may be desirable to administer ultrasound to a region for a period of time sufficient to cause the nanoparticle or liposomes to generate light. In some preferred embodiments, the ultrasound is not applied to a tissue for so long that significant toxicity to cloal tissues occurs from ROS generation by the sonosensitizer.

The emission of light from the photoexcited mechanoluminescent particle can be triggered by any suitable mechanical stimulus, e.g. by ultrasound. In some cases, the ultrasound is focused ultrasound. In some cases, the ultrasound signal has a frequency ranging from 150 kHz to 15 MHz, such as from 300 kHz to 5 MHz, or from 600 kHz to 2 MHz. The ultrasound signal can be repeated, i.e. wherein the signal is emitted for a time and then not emitted for a time, at any suitable repetition. In some cases, the ultrasound signal is repeated at a rate ranging from 0.05 repetitions per second to 20 repetitions per second, such as from 0.2 repetitions per second to 5 repetitions per second, or from 0.5 repetitions per second to 2 repetitions per second.

In some cases, the ultrasound signal has a spatial peak pulsed average intensity (ISPPA) at a target neuron ranging from 1 W/cm² to 100 W/cm², such as from 2 W/cm² to 50 W/cm², or from 5 W/cm² to 15 W/cm². As used herein, W/cm² refers to the units watts per centimeter square.

In some cases, the time interval between application of the ultrasound signal and the emission of light from the mechanoluminescent particle is 9 ms or less, such as 7 ms or less, 5 ms or less, or 3 ms or less. The subject can be, for example, a human, a primate, a rat, a mouse, a horse, a dog, or a cat.

### III. OPTOGENETIC METHODS

Optogenetics is a method that employs light to modulate tissue, such as neurons, that have been genetically modified to express light-sensitive proteins. In some cases, the neurons are neurons in the brain. Thus, by stimulating these brain neurons with light, brain processes and brain regions can be studied. In some cases, the photosensitive proteins are ion channels located in a cell membrane.

Brain studies with optogenetics traditionally employed, for example, fiber optics implanted into the brain to provide the excitatory light. However, such implantation causes various complications associated with the surgery and use, along with disadvantages such as infection risk and damage to brain tissue resulting from the surgery and implantation.

Any suitable photosensitive protein can be employed, such as channelrhodopsin-2 (ChR2), VChR1, iC++, ChRmine, or halorhodopsin (HPHR), e.g. a halorhodopsin from Natronomonas (NpHR). In some cases, the method also includes genetically modifying the neuron to express the photosensitive protein, e.g. with CRISPR gene editing.

The method can result, based on configuration, in the hyperpolarization or depolarization of the target neuron or neurons. Hyperpolarization includes partial hyperpolarization and complete hyperpolarization, whereas depolarization includes partial depolarization and complete depolarization.

### IV. FLUORESCENT IMAGING

Compositions and methods provided herein can allow for light emission deep within the body. The methods can be used in a deep-tissue photodynamic therapy. Such methods, which utilize a nanoparticle or liposome as disclosed herein, are distinct from functional ultrasound imaging that purely relies on ultrasound to directly image a part of the subject.

For example, the nanoparticle or liposome can produce light due to application of ultrasound in an internal region of the subject, such as the intestines of the patient. The emission of light from the particles (e.g., fluorescent light) can be measured in order to image the region. In some cases, the device measuring the fluorescent light is outside the patient, and in other cases it is inside the patient. For example, a fluorescent light measuring device can be inserted into the gastrointestinal tract, e.g. the intestines, of the patient, and can thereby measure the objects or features between the ultrasound stimulated mechanoluminescent particles and the detection device. In such cases, the mechanoluminescent particles can be administered to the subject in any suitable area. For example, if the intestines are to be imaged, the mechanoluminescent particles can be administered in the leg, and optionally photoexcited in the leg, where after they travel through veins to an intestine region, at which they are contacted with ultrasound signal.

### V. GENE EDITING

In some aspects, light generation via the nanoparticles and liposomes provided herein can be utilized in a gene editing method (e.g., deep-tissue gene editing mediated by ultrasound). These methods, which employ the nanoparticles or liposomes provided herein, are distinct from nonspecific gene editing that purely relies on systemically delivered CRISPR-Cas9 for editing the genome without spatiotemporal precision in the body.

As such, in some cases the tissue comprises a group of compounds that causes genetic modification to the tissue after absorbing the emitted light. In such cases, the method can be referred to as a method of selectively genetically modifying tissue by selectively applying an ultrasound signal to the tissue, i.e. surrounding tissues that do not receive the ultrasound signal or that do not comprise photoexcited liposomes or photoexcited nanoparticles are not genetically modified because they do not receive the light emitted by the nanoparticles or liposomes. The method can also be referred to as light-inducible gene editing. The gene editing can be referred to as spatially-selective gene editing since the gene editing will only occur in locations where the emitted light can penetrate.

For example, the photoexcited mechanoluminescent particle can be contacted with ultrasound in an internal region of the subject, such as the liver or the brain of the patient. In turn, the emission of light from the particles can be used to control the function of photoswitchable Cas9 in order to activate the CRISPR-Cas9 system for localized gene editing. In this case, the mechanoluminescent particles can be administered to the subject via intravenous injection. For example, if the genome in the liver is to be edited, the mechanoluminescent particles can be administered in the leg, and optionally photoexcited in the leg, where after they travel through veins to the liver, at which they are contacted with ultrasound signal to produce localized light emission and gene editing. Examples of such a photoswitchable genome editing methods are described by Moroz-Omori et al (ACS Central Science, 2020, 6, 5, 695, doi:10.1021/acscentrasci.9b01093) and Zhou et al (ACS Chemical Biology, 2018, 13, 2, 443, doi:10.1021/acschembio.7b00603), which are incorporated herein by reference.

### VI. PHARMACEUTICAL COMPOSITIONS

In some embodiments, the nanoparticles (e.g., lipid nanoparticles) or liposomes of the present disclosure are included in a pharmaceutical composition. Pharmaceutical compositions of the present invention comprise an effective amount of one or more compounds of the present disclosure, e.g., a mechanoluminescent nanoparticle or mechanoluminescent liposome, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains a mechanoluminescent nanoparticle or mechanoluminescent liposome as described herein or additional active ingredient will be known to those of skill in the art in light of the present disclosure, as exemplified by Remington: The Science and Practice of Pharmacy, 21^{st} Ed., Lippincott Williams and Wilkins, 2005, incorporated herein by reference. Moreover, for animal (e.g., human) administration, it will be understood that preparations should typically meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington—23rd Edition, October 2020, incorporated herein by reference). Except insofar as any conventional carrier is incompatible with the active ingredient, its use in the pharmaceutical compositions is contemplated.

The mechanoluminescent nanoparticle or mechanoluminescent liposome may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), via injection, infusion, continuous infusion, or localized perfusion bathing target cells directly.

### VII. SYSTEMS

Provided are systems for contacting a tissue of a subject with light, wherein each system comprises: an ultrasound device configured to apply an ultrasound signal to a mechanoluminescent nanoparticle or mechanoluminescent liposome as described herein (wherein the nanoparticle or liposome comprises a sonosensitizer and a chemiluminescent compound) in proximity to the tissue, thereby causing the nanoparticle or liposome to emit light that contacts the tissue. The ultrasound emitting device can have any of the features or properties described above, e.g. it can have a frequency ranging from 150 kHz to 15 MHz and it can be focused ultrasound.

In some cases, the system further includes an apparatus comprising a nucleic acid comprising a nucleotide sequence encoding the photosensitive protein, e.g. the photosensitive protein that can be used for optogenetics, as described herein. For instance, the apparatus can be a syringe that contains an aqueous liquid comprising the nucleic acid.

### VIII. KITS

Provided are kits that can be employed to perform the methods described herein. In some cases, the kit includes two or more of: a nanoparticle or liposome comprising a sonosensitizer and a chemiluminescent compound; a device for administering the nanoparticles or liposomes to a subject; a device for emitting light onto an external surface of a subject such that a plurality of the nanoparticles or liposomes in the subject are photoexcited; and a device for emitting ultrasound. In some cases, the kit includes the nanoparticles or liposomes and the device for emitting ultrasound. In some cases, the kit further includes a device for emitting light onto an external surface of a subject such that a mechanoluminescent nanoparticle or mechanoluminescent liposome in the subject is photoexcited, a device for administering the nanoparticles or liposomes to a subject, or both.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Ultrasound Triggered Liposome Light Source for Noninvasive Optogenetics

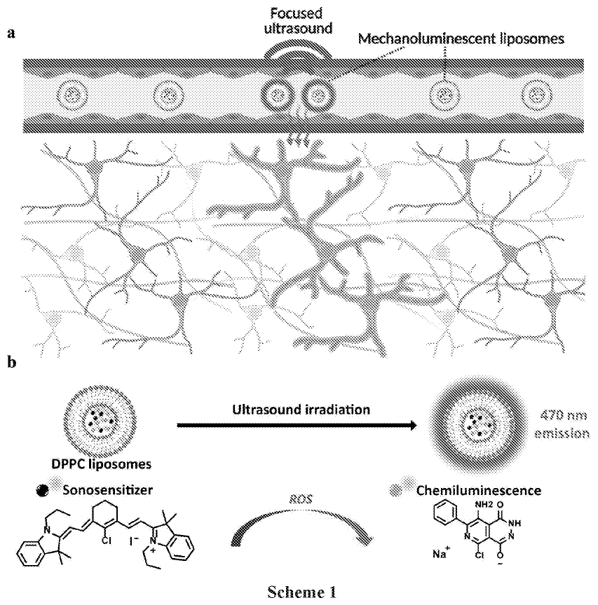

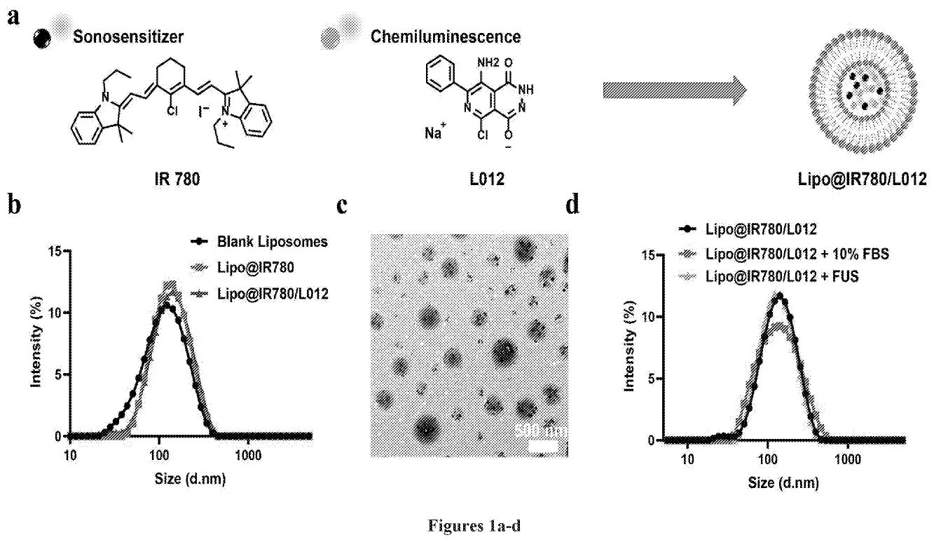

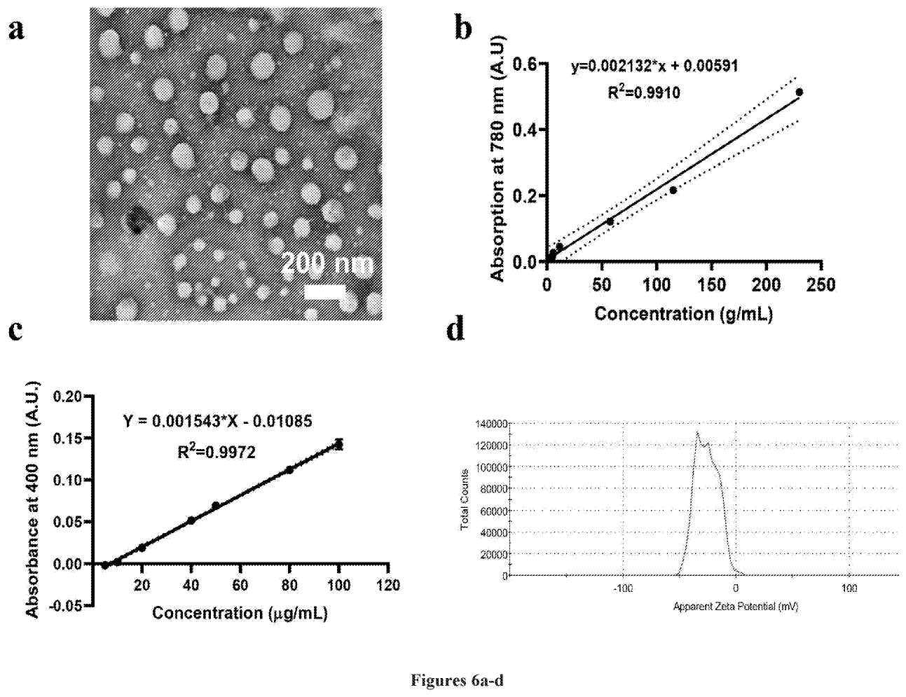

To prepare the FUS triggered nanoscopic light source, liposomes were first prepared. The unilamellar vesicles (FIG. 6a) were prepared through a thin film hydration strategy (Wang et. al., 2021). Then, L012 and IR 780 were loaded into the vehicles to fabricate the FUS triggered nano light source (Lipo@IR780/L012) (FIG. 1a). IR780 loaded liposomes (Lipo@IR780) exhibited negligible size differences compared with blank liposomes, as shown in FIG. 1b and Table 1. Transmission electron microscopy (TEM) showed a uniform spherical shape of Lipo@IR780/L012 (FIG. 1c) compared with the typical liposome morphology of blank lipid vehicles (FIG. 6a) due to the integration of IR780/L012. We evaluated the stability of Lipo@IR780/L012 nanoparticles via dynamical light scattering (DLS) in body fluid mimic solution and FUS irradiation. There were no obvious size changes after incubation in 10% fetal bovine serum containing solution or FUS irradiation (FIG. 1d), indicating excellent stability of Lipo@IR780/L012 nanoparticles in blood circulation and under FUS. The drug loading content (DLC) of IR780 (˜4.6 wt. %) and L012 (˜4.3 wt. %) was measured by the UV-Vis spectrum according to the calibration curves (FIGS. 6b and 6c) and indicated the efficient drug loading capacity of the lipid vehicles. The Lipo@IR780/L012 nanoparticles with an average diameter of about 120 nm (FIG. 1b, 1d and Table 2) and negative surface zeta potential (FIG. 6f and Table 2) were good for circumventing rapid clearance via reticuloendothelial system, liver and kidney after intravenous administration (Wang and Liu, 2021; Tsoi et. al., 2016).

TABLE 2
The details of various liposomes determined via DLS and UV-Vis.

| | | Size | | Zeta | DLC (wt %) | DLC (wt %) |
|---|---|---|---|---|---|---|
| entries | Nanoparticles | (d · nm) | PDI | potential (mv) | of IR780 | of L012 |
| 1 | Blank Lipo | 105.0 ± 3.7 | 0.228 | −28.0 ± 1.3 | N/A | N/A |
| 2 | Lipo@IR780 | 119.3 ± 0.2 | 0.183 | −24.1 ± 0.5 | 4.63 ± 0.59 | N/A |
| 3 | Lipo@IR780/L012 | 120.9 ± 0.5 | 0.189 | −25.8 ± 0.7 | 4.61 ± 0.66 | 4.30 ± 0.44 |
| 4 | Lipo@IR780/L012 + 10% FBS | 119.7 ± 0.8 | 0.196 | N/A | N/A | N/A |
| 5 | Lipo@IR780/L012 + FUS | 120.2 ± 0.2 | 0.188 | N/A | N/A | N/A |

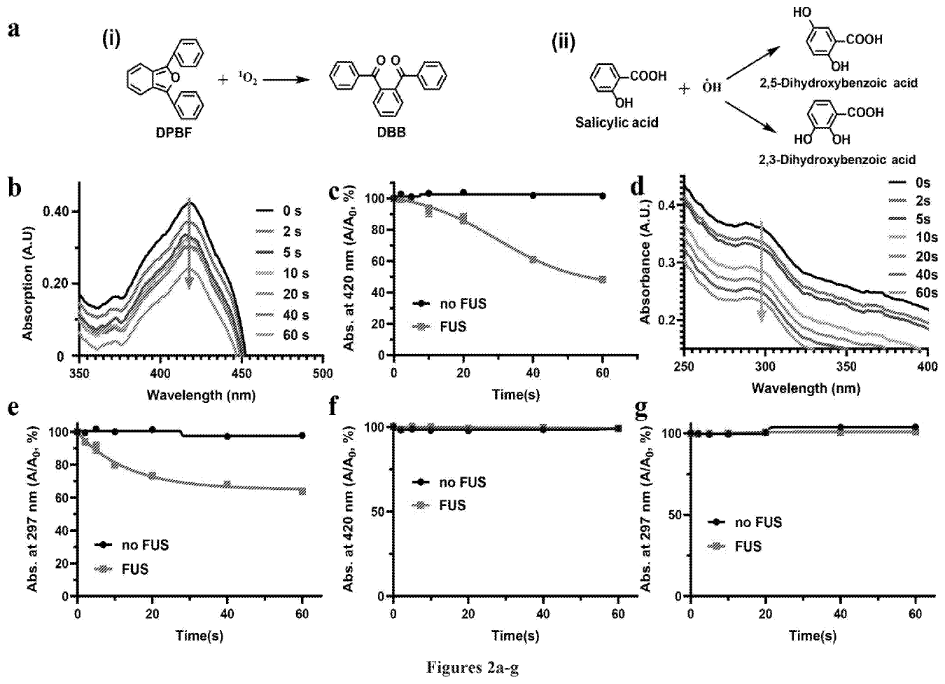

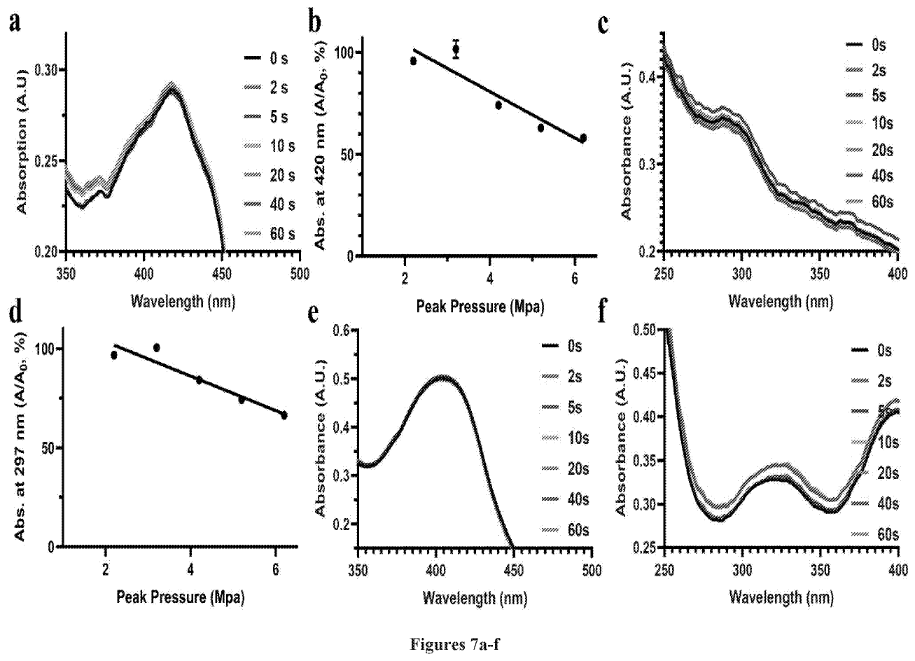

As the primary trigger of the Lipo@IR780/L012 nano light source, ROS are crucial in activating the system through a rapid reaction with L012. Thus, the types of generated ROS were investigated in Lipo@IR780 nanoparticles. 1,3-diphenylisobenzofuran (DPBF) and salicylic acid (SA) probes were adopted to specifically detect the generation of ¹O₂ and ·OH under FUS irradiation (FIG. 2a). DPBF possesses highly specific reactivity towards ¹O₂, forming 1,2-dibenzoylbenzene (DBB) as shown in FIG. 2a-I (Gomes et. al., 2005; Wang et. al., 2018; Entradas et. al., 2020). Lipo@IR780 containing DPBF solution was irradiated under different durations of FUS (1.5 MHz, 6.2 MPa). As shown in FIG. 2b, the characteristic UV-vis absorption peak of DPBF at 420 nm dramatically decreased with the extension of FUS time due to the decomposition of DPBF in presence of ¹O₂, while no obvious changes were seen without FUS irradiation (FIG. 7a). The quantification determined that around 52% DPBF was decomposed after 60 s FUS irradiation, but there was no DPBF consumption without FUS irradiation (FIG. 2c). Notably, the generation of ¹O₂ was an FUS power dependent process, which was positively related to FUS peak pressure (FIG. 7b) when peak pressure was above 2.2 MPa (no ¹O₂ generation below 2.2 MPa). Similarly, SA was used to track the generation of ·OH from Lipo@IR780. SA specifically scavenges ·OH to form 2,3-dihidroxybenzoic acid and 2,5-dihydroxibenzoic acid (FIG. 2a-ii). We did not observe obvious UV-Vis intensity changes of Lipo@IR780 at 297 nm without FUS irradiation (FIG. 2f and FIG. 7c). However, the UV-Vis intensity at 297 nm gradually decreased with the FUS irradiation time due to the decomposition of SA, and around 36% SA reacted with ·OH after 60 s irradiation (FIG. 2f). In addition, the generation of ·OH was also linearly dependent on FUS peak pressure (FIG. 7d). Although ROS plays essential roles in various biological processes, it would directly kill cells by oxidation at high levels (Wang and Yi, 2008; Mittler, 2017). Thus, we further evaluated the concentrations of ·OH and ¹O₂ after liposome encapsulation in Lipo@IR780/L012. The results showed that there were no ROS residues under FUS irradiation in Lipo@IR780/L012 (FIG. 2f-2g, and FIG. 7e-7f), since the generated ROS were rapidly consumed by L012 to emit blue light.

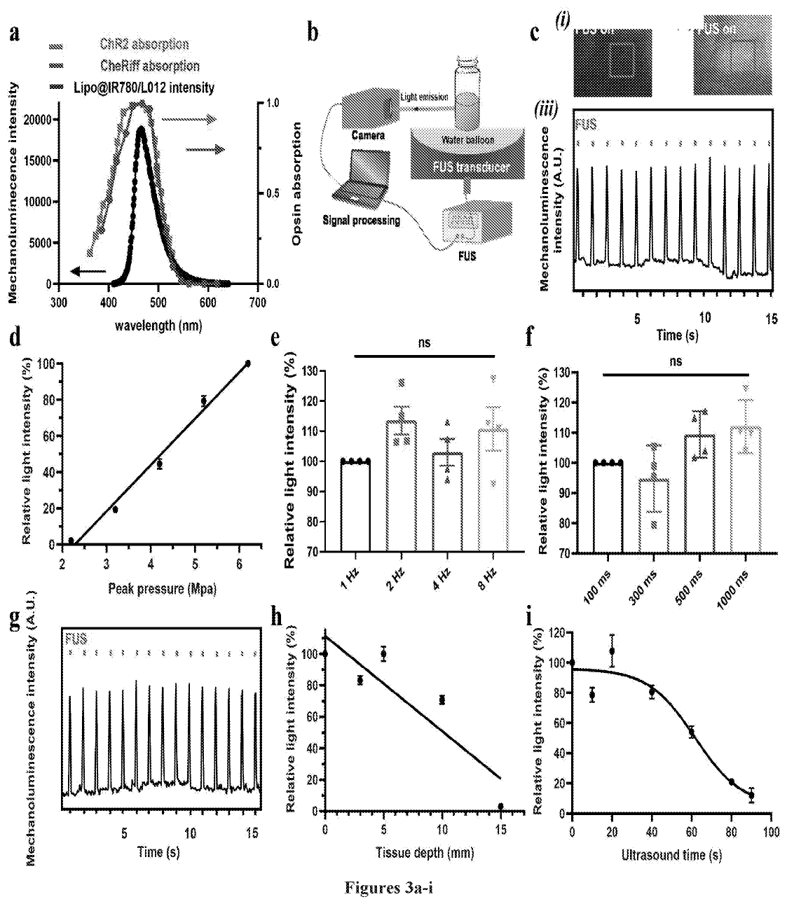

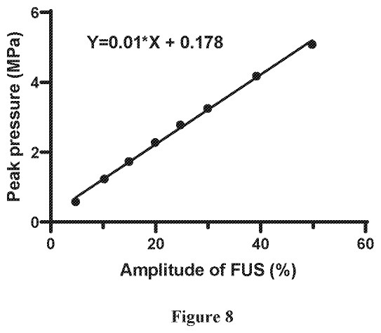

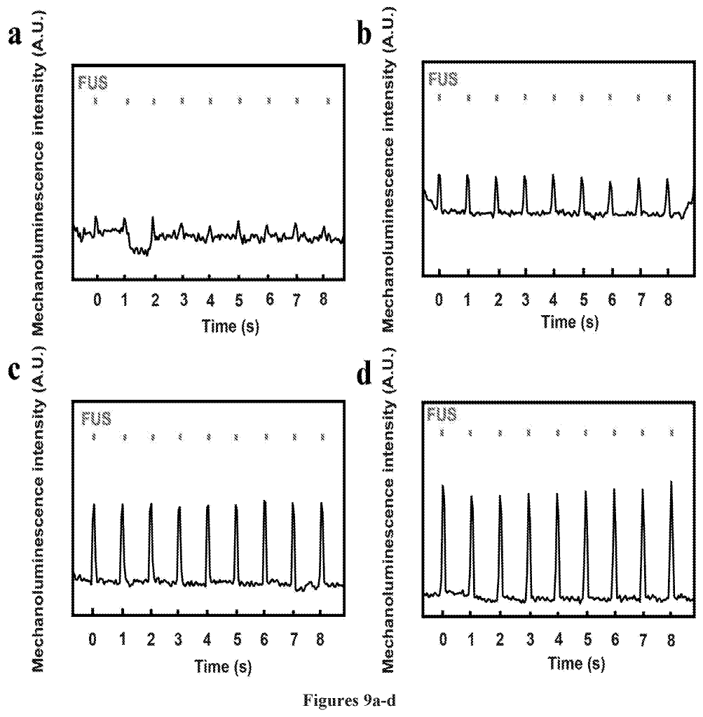

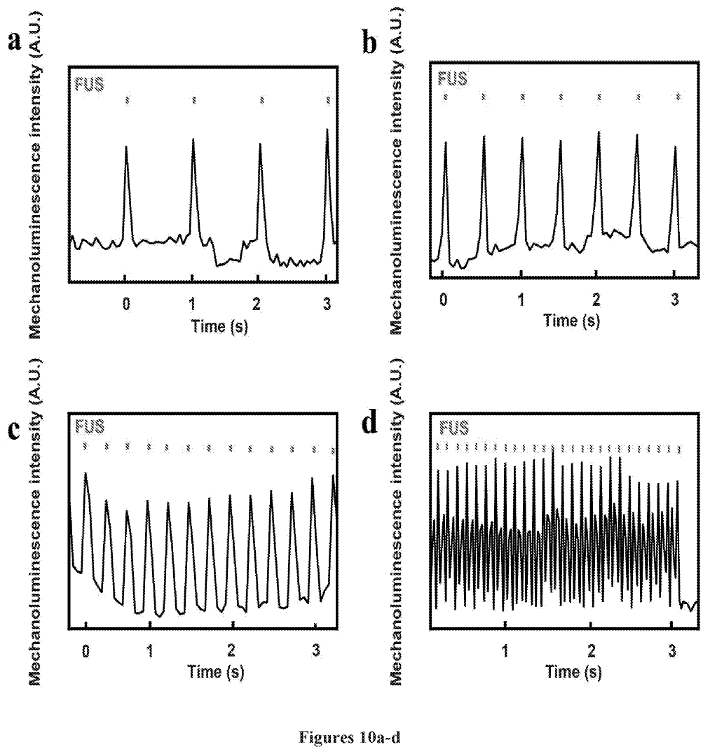

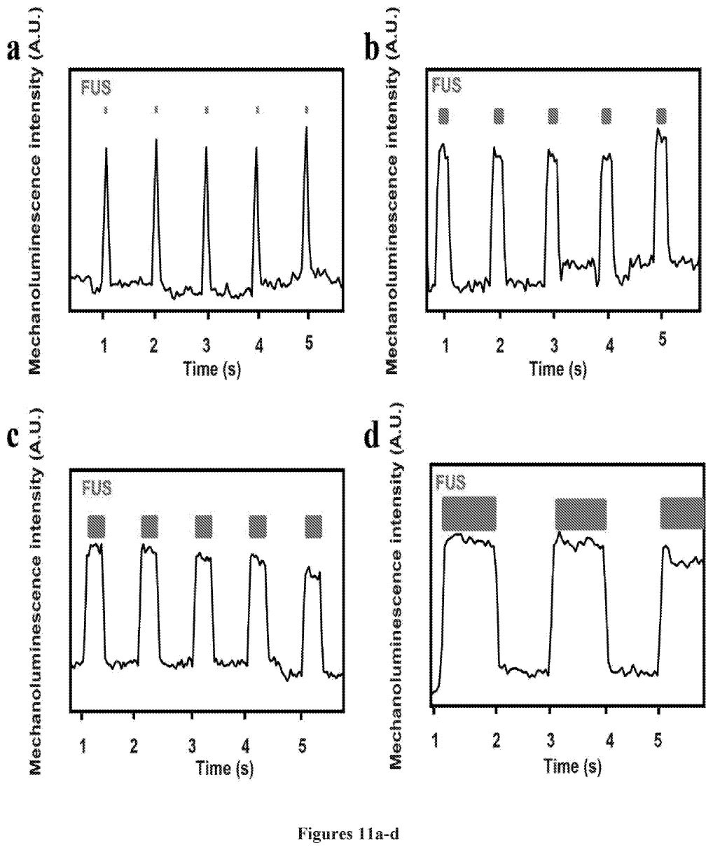

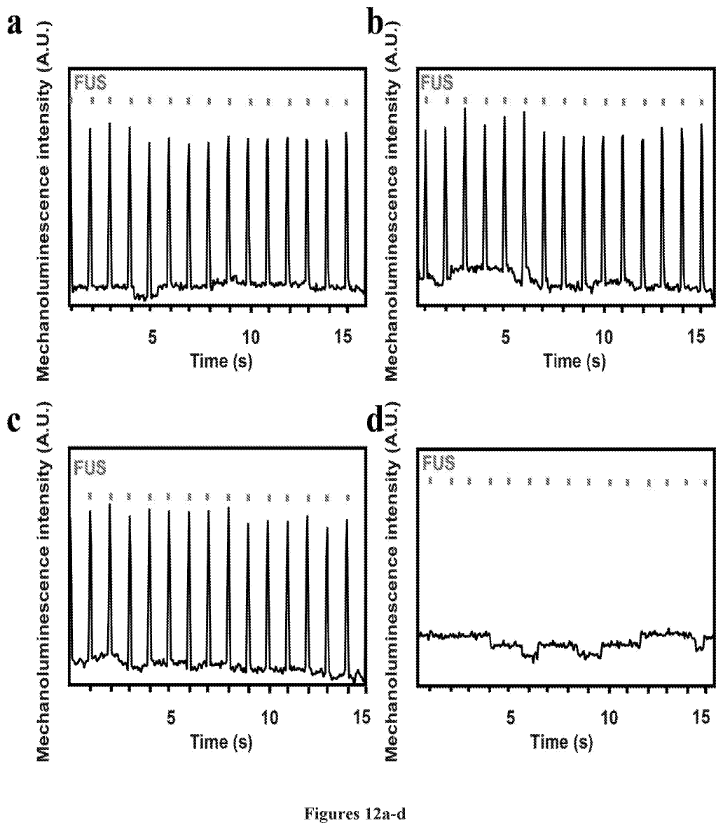

Next, the mechanoluminescence performance of the Lipo@IR780/L012 system was investigated. The mechanoluminescence spectra of Lipo@IR780/L012 showed the maximal emission peak was around 470 nm, (FIG. 3a) which highly overlaps with the channelrhodopsin-2 (ChR2) absorption spectrum and suggests that Lipo@IR780/L012 is suitable to activate ChR2 for optogenetic stimulation (Zhang et. al., 2007). We also detected the light emission from Lipo@IR780/L012 through a photon processing system in real time (FIG. 3b). Time-resolved sonoluminescence spectra revealed sharply increased photon density was detected upon FUS irradiation (1.5 MHz, pulse 100 ms on 900 ms off, 6.2 MPa) from the baseline of Lipo@IR780/L012 nanoparticles (FIG. 3c), where the ultrasound peak pressure was calculated via amplitude calibration (FIG. 8). Of note, the photon density was positively and linearly correlated with peak pressure, and no photons were detected when peak pressure was below 2.2 MPa (FIG. 3d and FIG. 9) which is consistent with the FUS peak pressure dependent ROS generation process of Lipo@IR780/L012 nanoparticles (FIGS. 7b and 7d). This shows that the rate determining step of light emission from Lipo@IR780/L012 nanoparticles was ROS concentration, since the light emission intensity exhibited a positive relation with FUS peak pressure. High temporal resolution of light emission upon FUS irradiation are important for achieving precise control over specific neuronal activity through sono-optogenetics. Therefore, we further evaluated the FUS triggered photon emission at different irradiation frequencies (1.5 MHz, 6.2 MPa) (FIG. 10). The system could still exhibit high synchronism at 8 Hz irradiation and there were no photon density changes with the irradiation frequencies (FIG. 3e and FIG. 10). We also changed the irradiation pulses from 100 ms to 1000 ms and the nanoparticles showed excellent stability to output photons and there were no influences on light intensity (FIG. 3f and FIG. 11). Next, we evaluated the noninvasive activation of Lipo@IR780/L012 nanoparticles using pork skin to mimic the normal brain tissue. As shown in FIG. 3g and FIG. 12, remarkable emitted light intensity was achieved from Lipo@IR780/L012 nanoparticles even at a tissue depth around 10 mm. Specifically, the light intensity only decreased around 30% (FIG. 3h) at a depth of 10 mm compared with no tissue covered group but reduced more significantly at a tissue depth over 15 mm due to the dissipation and scattering of ultrasound energy into surrounding tissue (Holt and Roy, 2001). Moreover, continuous and effective light emission is crucial for long-lasting optogenetics stimulation. Therefore, we also evaluated the light emission half-time of Lipo@IR780/L012 nanoparticles under FUS irradiation. As shown in FIG. 3i, the decay half-time of light intensity was around 60 s (1.5 MHz, pulse 1000 ms on 1000 ms off, 6.2 MPa). The light intensity gradually decreased with irradiation time due to the continuous and irreversible consumption of L012 until it reached around 10% after 90 s irradiation. All these results demonstrate that the Lipo@IR780/L012 system demonstrated excellent reliability and synchronism for repeated, non-invasive sono-optogenetics stimulation.

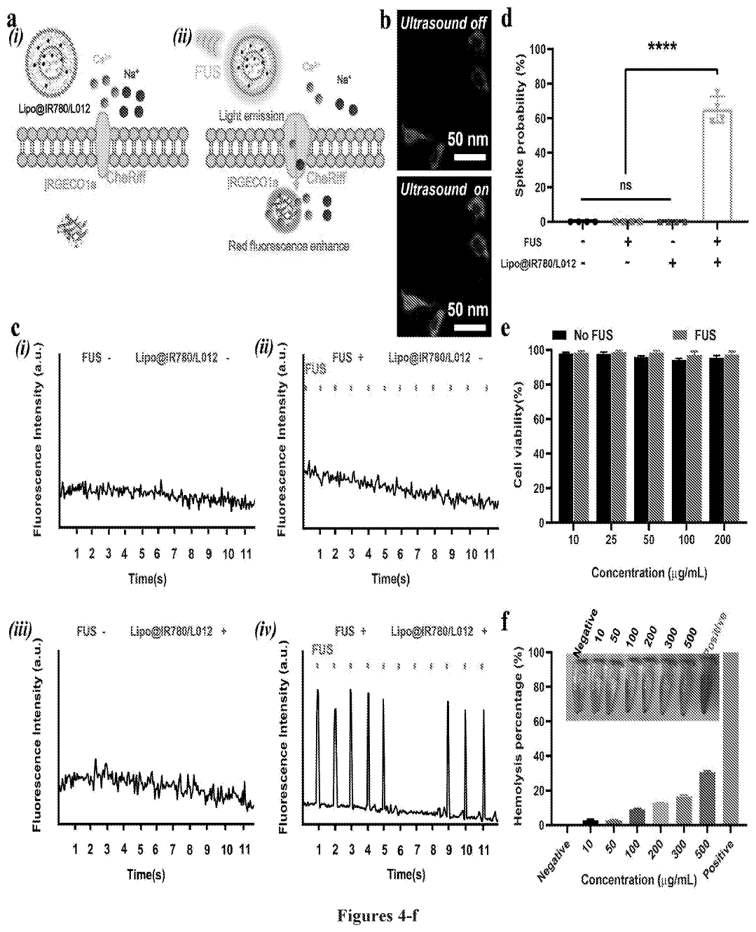

In order to evaluate the activation of opsins under repetitive FUS irradiation, the inventors utilized CheRiff-eGFP tet-on spiking HEK cells with constitutively expressed blue light-activated CheRiff actuator, voltage-gated sodium channel Na_{V}1.5, and inducibly (tet-on) expressed Kᵢᵣ2.1. The mechanoluminescence spectra of Lipo@IR780/L012 also highly overlapped with CheRiff opsin absorption spectra (FIG. 3a) (Hochbaum et. al., 2014). The spiking HEK cells were transfected with pGP-CMV-NES-jRGECO1a plasmids to express calcium ion (Ca²⁺) indicators, as shown in FIG. 4a. Once the opsins channels were activated under the irradiation of blue light, calcium ions would rapidly diffuse into the cells and then bind with the jRGECO1a calcium indicators for increased red fluorescence. Our experiments confirmed that the red fluorescence signal remarkably increased after the spiking HEK cells with Lipo@IR780/L012 nanoparticles were irradiated by ultrasound (FIG. 4b). Time-resolved red fluorescence signal revealed that the sharp increment was observed only in the Lipo@IR780/L012 nanoparticles (+) group with FUS irradiation, but no changes in all other control groups without ultrasound or nanoparticles (FIG. 4c), which demonstrates the spiking HEK cells only fired when the mechanoluminescence occurred. Of note, the red fluorescence signal gently decayed with the continuous irradiation due to the photobleaching of jRGECO1a calcium indicators. In addition, mechanoluminescence power would decrease with the continuous consumption of L012 under the FUS irradiation, thus influencing the spike probability. As shown in FIG. 4d, the spike probability was around 66% under the mechanoluminescence irradiation. We then evaluated the biosafety and biocompatibility of Lipo@IR780/L012 nanoparticles. As shown in FIG. 4e, the cell viability tests in human embryonic kidney 293 (HEK) cells determined that the minimum toxicity to the cells even when the Lipo@IR780/L012 nanoparticles concentration was up to 200 μg/mL, and there was no obvious toxicity to HEK cells after FUS stimulation due to the high stability of Lipo@IR780/L012 nanoparticles and minimum ROS residue leakage from the liposomes. The hemolysis assay shown in FIG. 4f also determined that the Lipo@IR780/L012 nanoparticles exhibited excellent biocompatibility, with only around 30% hemolysis occurred at a concentration around 500 μg/mL. These results indicate that the Lipo@IR780/L012 nanoparticles are safe enough for further in vivo application.

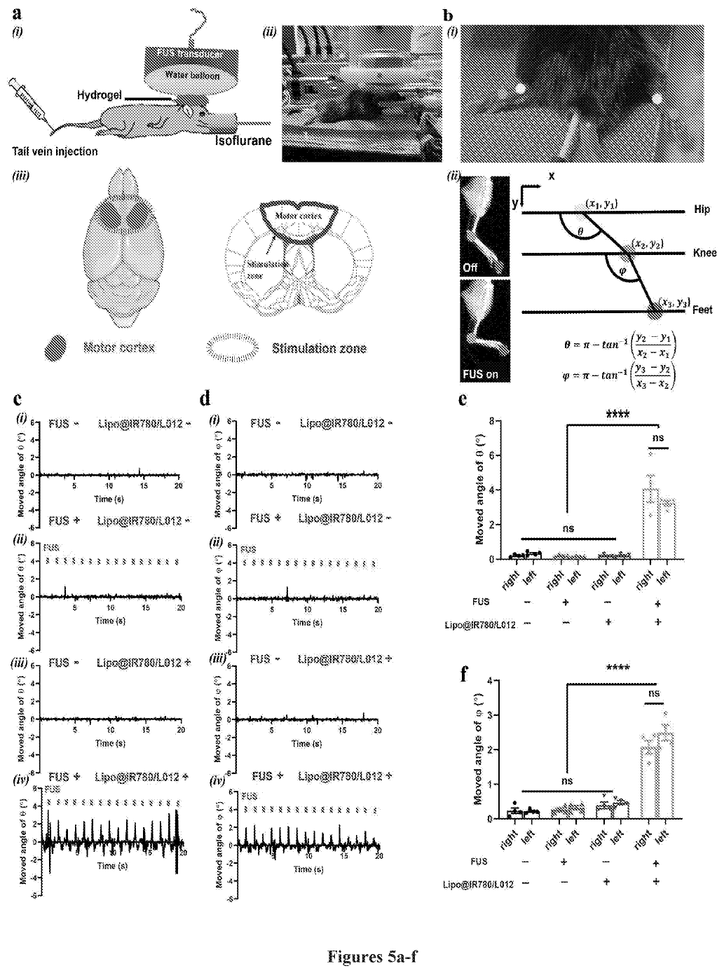

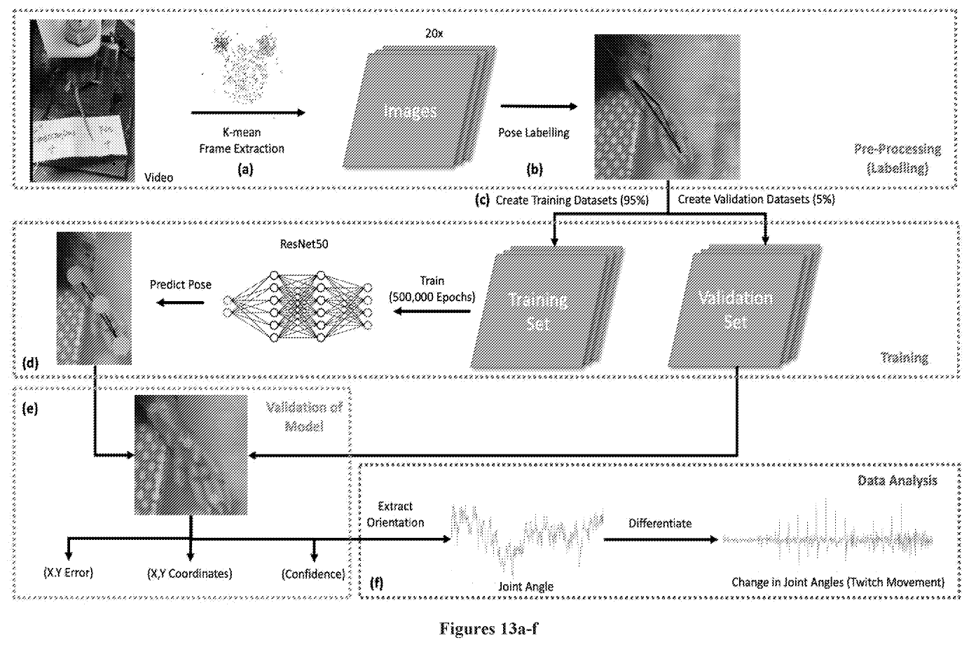

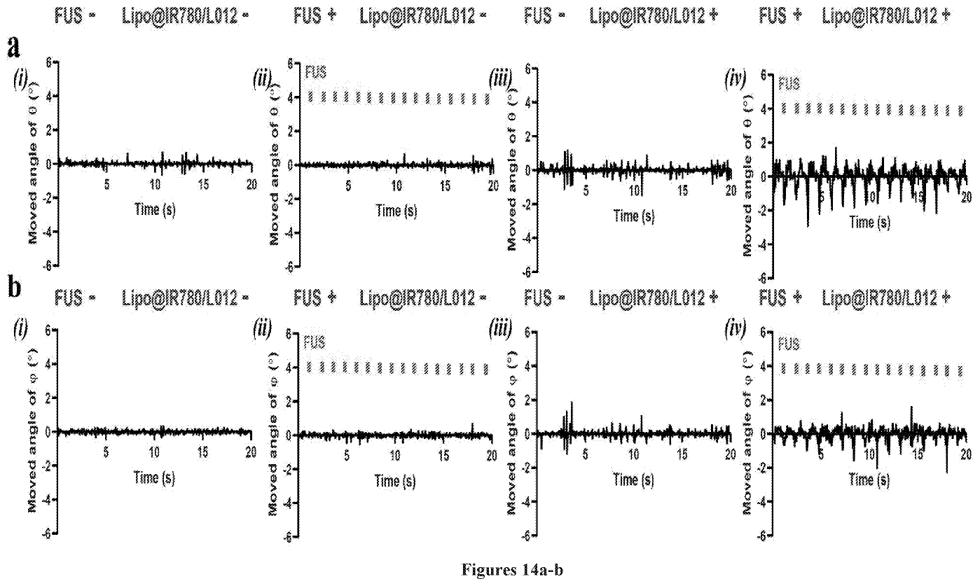

The in vitro tests demonstrated the opsins are effectively activated under the ultrasound irradiation with Lipo@IR780/L012 nanoparticles. Following this, we asked whether the Lipo@IR780/L012 nanoparticles allowed for noninvasive optogenetic brain stimulation in ChR2-expressing mice after tail vein administration under FUS irradiation. Thy 1-ChR2-YFP transgenic mice with ChR2 expressing neurons were used for in vivo sono-optogenetic stimulation. As shown in FIG. 5a, the mouse was head-fixed in a stereotaxic frame and anesthetized with 2.5% isoflurane. Then, Lipo@IR780/L012 nanoparticles at a concentration of 10 mg/mL were injected through the tail vein. The FUS transducer water balloon was placed in direct contact with the intact scalp of the mouse with filling ultrasound gel (FIG. 5a-i and 5a-ii). To visually evaluate sono-optogenetic brain stimulation, the motor cortex areas were irradiated (FIG. 5a-iii), since the motor cortex is responsible for controlling the execution of body movement, including the complex movements of the leg and fingers, allowing us to easily evaluate activation by tracking mouse movement (Sanes and Donoghue, 2000). After i.v. administration off 15 minutes, the isoflurane concentration was decreased to 0.5% to make sure the mice were in light anesthesia status in order to effectively observe its response to FUS irradiation (1.5 MHz, pulse 100 ms on 900 ms off, 6.2 MPa). Camera video was used to track the synchronized limbs' response under the sono-optogenetic stimulations, where the hip, knee and feet were marked with different color dots (FIG. 5b). Kinematic data were obtained by using DeepLabCut to quantify the joint angle (Hip to knee: θ, and knee to feet: φ, shown in FIG. 5b ii) changes under the ultrasound irradiation (FIG. 13). Our results revealed sharp changes in θ in the Lipo@IR780/L012 nanoparticles (+) group with FUS irradiation, but no change in all other groups in left and right limbs (FIG. 5c and FIG. 14). Similar results were also observed in knee to feet movement (φ), indicating the temporary and reversible activation of motor cortex neurons under the repetitive sonoluminescence irradiation. The quantitative analysis of θ (FIG. 5e) and φ (FIG. 5f) angle changes showed that there were statistically significant differences between Lipo@IR780/L012 nanoparticles (+)/FUS irradiation (+) groups and other control groups with Lipo@IR780/L012 nanoparticles (+)/FUS irradiation (−), Lipo@IR780/L012 nanoparticles (−)/FUS irradiation (+) and Lipo@IR780/L012 nanoparticles (−)/FUS irradiation (−). These results revealed that our FUS induced Lipo@IR780/L012 system can be used to achieve highly reliable noninvasive brain stimulation.

As shown above, a biocompatible mechanoluminescence system was developed using organic lipid vehicles, sonosensitizer IR780 and chemiluminescence L012 to achieve noninvasive optogenetic stimulation of neural activity under focused ultrasound, without the need for charging of the nanoparticles. Ultrasound energy is noninvasively transmitted to deep brain tissue and sensed via the sonosensitizer IR780, generating ROS that triggers the nearby chemiluminescence L012, resulting in blue light emission. These Lipo@IR780/L012 nanoparticles were able to emit blue light under FUS sufficient for activating the CheRiff expressed spiking HEK cells. Furthermore, in vivo experiments demonstrated that motor cortex neurons in Thy1-ChR2-YFP transgenic mice can be temporarily and reversibly activated under the repetitive FUS irradiation after i.v. injection of Lipo@IR780/L012, thus achieving noninvasive sono-optogenetic brain stimulation.

### Example 2

### Materials and Methods

The following materials and methods were used in the experiments performed in Example 1.

Materials: 1,2-dipalmitoyl-sn-glycero-3-phosphocholine (DPPC), cholesterol, 1,2-distearoyl-sn-glycero-3-phosphoethanolamine-N-[amino(polyethylene glycol)-2000) (DSPE-PEG2000), IR-780 iodide (IR-780), L012 sodium salt (L012), 1,3-Diphenylisobenzofuran (DPBF), salicylic acid (SA) were ordered from Sigma-Aldrich. pGP-CMV-NES-jRGECO1a was a gift from Douglas Kim & GENIE Project (Addgene plasmid # 61563; http://n2t.net/addgene: 61563; RRID: Addgene 61563). CheRiff_eGFP tet-on spiking HEK cells were purchased from ATCC.

Characterization: FEI TECNAI G2 F20 X-TWIN Transmission Electron Microscope (TEM) was used to evaluate the morphology of liposomes. The hydrodynamic diameter and surface potential of liposomes were evaluated through dynamic light scattering (DLS, Zetasizer Nano-ZS from Malvern Instruments). The UV-vis spectrum was measured via Eppendorf Biospectrometer. The sonoluminescence spectrum was evaluated through Fluorolog3 Fluorimeter. The focused ultrasound (FUS) equipment was purchased from Image Guided Therapy. The fluorescence images and videos were recorded by Leica DMi8 fluorescence microscope.

Preparation of liposomes. Thin film hydration strategy was used to prepare the liposomes (Li et. al., 2022; Jiang et. al., 2021). In brief, 40 mg DPPC, 10 mg cholesterol, 4 mg DSPE-PEG₂₀₀₀ and 3 mg IR 780 were dissolved in 10 mL mixed solution (chloroform: methanol, 5/1, v/v). The solvents were removed via vacuum distillation at 50° C. for 1 h to make thin film in 100 mL flask. Then, 5 mL (NH₄)₂SO₄ solution (200 mM) was added to dissolve the film. The mixture was sonicated at 60° C. water bath for 10 min to make sure the film was totally dispersed in solution, and extruded five times through 0.22 μm polycarbonate filter to homogenize the primary liposomes solution. Firstly, to prepare IR780 loaded liposomes (Lipo@IR780), the above solution was dialyzed against PBS (pH 7.4, 1 L) for 6 h to obtain Lipo@IR780, where the PBS was replaced each 2 h. In addition, to prepare IR780 and L012 coloaded liposomes (Lipo@IR780/L012), 5 mL Lipo@IR780 solution was incubated with L012 (1 mg/mL) at 60°° C. for 30 min. Then, the mixture was dialyzed against 1 L PBS (pH 8.5) for 4 h to remove uploaded L012, where the PBS was replaced each 2 h. 1 mL Lipo@IR780 and 1 mL Lipo@IR780/L012 was lyophilized to calculate the drug loading content of IR780/L012 through UV-Vis calibration curve (FIGS. 6d and 6e). 1 mg/mL liposome solution was used to prepare TEM samples and for the DLS tests, including the hydrodynamic size and zeta potential.

Stability tests of liposomes. 10% FBS solution was used to mimic the body fluid. Lipo@IR780/L012 liposomes (1 mg/mL) were mixed with 10% FBS solution, after incubated at 37° C. for 4 h, the solution was tested via DLS to evaluate the stability. In addition, to evaluate the stability of Lipo@IR780/L012 under the FUS stimulation, 1 mg/mL Lipo@IR780/L012 solution was treated under the FUS irradiation (1.5 MHz, 6.2 MPa) for 60 s, and then tested the size via DLS.

Detection of the generation of ¹O₂. 1 mL Lipo@IR780 (1 mg/mL) or Lipo@IR780/L012 (1 mg/mL) with 30 μL 1 mg/mL DPBF (dissolved in methanol) were mixed. The UV-Vis characteristic absorption peak of DPBF at 420 nm was used to track the generation of ¹O₂. The mixture was treated with or without FUS irradiation (1.5 MHz, 6.2 MPa), and extracted out 10 μL for UV-Vis spectrum tests. The absorbance change of DPBF at 420 nm was used to quantify the generation of ¹O₂. Similarly, in order to quantify the generation of ¹O₂ under the different FUS powder density, we treated the mixture at different amplitudes (amplitude from 0˜6.2 MPa at 1.5 MHz).

Detection of the generation of ·OH. 1 mL Lipo@IR780 (1 mg/mL) or Lipo@IR780/L012 (1 mg/mL) with 50 μL 1 mg/mL salicylic acid (SA) dissolved in methanol were mixed. SA is a typical probe for detection of ·OH. The UV-Vis characteristic absorption peak of SA at 297 nm was used to track the generation of ·OH. The mixture was treated with or without FUS irradiation (1.5 MHz, 6.2 MPa), and extracted out 10 μL for UV-Vis spectrum tests at fixed time. The absorbance change of SA at 297 nm was used to quantify the generation of ·OH. Similarly, in order to quantify the generation of ·OH under the different FUS powder density, we treated the mixture at different amplitudes (amplitude from 0˜6.2 MPa at 1.5 MHz).

Mechanoluminescence spectrum of Lipo@IR780/L012. 30% hydrogen peroxide was used to mimic the reactive oxygen species (ROS), where 1 mL Lipo@IR780/L012 (10 mg/mL) was mixed with 30% hydrogen peroxide (0.1 mL), and the mechanoluminescence spectrum was measured via Fluorolog3 Fluorimeter. ChR2 and ChiReff absorption spectra were referred from previous work (Zhang et. al., 2007; Hochbaum et. al., 2014), and extracted via GetDataGraphDigitizer software.

FUS triggered blue light emission from Lipo@IR780/L012 solution. 1 mL Lipo@IR780/L012 solution (10 mg/mL) was added into a 2 mL vial. The vial was fixed with iron support and touched with the water balloon of the FUS transducer (1.5 MHz). To avoid air bubbles, ultrasound gel was used to fill the gaps between the vial and water balloon. First, FUS was applied with a repetition frequency of 1 Hz with FUS pulse of (100 ms on, 900 ms off), and the amplitude was changed from 2.2 MPa to 6.2 MPa to evaluate the influence of FUS power on light emission. Then, we fixed the FUS amplitude at 6.2 MPa, and change the FUS pulse time from 100 ms on, 300 ms on, 500 ms on to 1000 ms on, all with 1000 ms off. Finally, we also changed the FUS repetition frequency at 1 Hz (50 ms on, 950 ms off), 2 Hz (50 ms on, 450 ms off), 4Hz (50 ms on, 200 ms off) and 8 Hz (50 ms on, 75 ms off) at 6.2 MPa. The blue light emission was recorded through a monochrome CMOS camera (CS165MU1/M—Zelux® 1.6 MP Monochrome CMOS Camera, Throlabs). All the parameters were fixed to record the video, and the data were analyzed by Image J software.

Deep tissue penetration evaluation of Lipo@IR780/L012 solution. Pork skin was used to mimic the normal tissue. Different depth pork skin (3 mm, 5 mm, 10 mm and 15 mm) was firstly placed in the water balloon with ultrasound gel covered, respectively. The vial loaded 1 mL Lipo@IR780/L012 solution (10 mg/mL) was then placed in the pork skin filling with ultrasound gel. After totally removing the bubbles and gaps, FUS was used to irradiate the nanoparticle solution (1.5 MHz, FUS pulse 100 ms on, 900 ms off, 6.2 MPa). The emission was recorded through a monochrome CMOS camera for further analysis.

Blue light emission half-time evaluation of Lipo@IR780/L012 solution. 2 mL vial with 1 mL Lipo@IR780/L012 solution (10 mg/mL) was placed in the water balloon. After totally removing the bubbles and gaps, FUS was used to irradiate the nanoparticle solution (1.5 MHz, pulse 1000 ms on, 1000 ms off, 6.2 MPa) until no light emission. The emission was recorded through a monochrome CMOS camera, and the data was analyzed by Image J software.

Hemolysis tests of Lipo@IR780/L012 nanoparticles. The experiments were conducted according to the previous method (Wang et. al., 2021). Briefly, 0.5 mL fresh blood was obtained from the mouse heart and added into 10 mL PBS. The mixture was centrifuged at 8000 rpm/min for 5 min and washed the pallets with fresh PBS three times to obtain the red blood cells (RBCs). After that, the RBCs were resuspended with 2 mL PBS. 1 mL Lipo@IR780/L012 nanoparticles solution with different concentrations was mixed with 20 μL RBCs solution, and incubated at 37° C. for 2 h. Of note, 20 μL RBCs solution was added into 1 mL DI water as positive control and 1 mL PBS as negative control. Then, the mixture was centrifuged at 12000 rpm/min for 5 min. The supernatant was collected to measure the absorption intensity at 541 nm (microplate reader, BioTek Synergy H1). The hemolysis percentage was calculated according to the following equation:

$$Hemolysis percentage ( \% ) = Absorbance of samples / absorption of positive control \times 100 \%$$

Cell viability tests of Lipo@IR780/L012 nanoparticles. Human embryonic kidney 293 (HEK293T) cells were seeded into 96 well plates coated with 10 μg/mL Poly-L-Ornithine solution. After the confluency was around 90%, complete medium containing Lipo@IR780/L012 nanoparticles at various concentrations were added into the plate. Moreover, we also evaluated the cell viability of FUS treated Lipo@IR780/L012 nanoparticles. The Lipo@IR780/L012 solution was treated via FUS (1.5 MHz, 6.2 MPa) for 60 s, after that, the Lipo@IR780/L012 was added to complete medium with different concentrations and added into the plates. After incubation for 24 h, 10 μL of Cell-Titer blue reagent (Promega Corporation) was added into each well. Cells were incubated for another 4 h for fluorescence absorption tests cia microplate reader (BioTek Synergy H1, 560ex/590em nm). The cell viability was calculated according to the following equation (Wang et. al., 2016; Wang et. al., 2017).

$$Cell viability ( \% ) = flourescence intensity of sample / flourescence intensity of control \times 100 \%$$

In vitro sono-optogenetic. CheRiff_eGFP tet-on spiking HEK cells with constitutive expressed blue light-actived channel rhodopsin actuator, voltage-gated sodium channel Nav1.5, and inducibly (tet-on) express Kir2.1 were purchased from American Type Culture Collection (ATCC). The cells were cultured in Dulbecco's Minimal Essential Medium (DMEM) with 10% FBS and 2 mM L-glutamine, and seed into the 24 well plates coated with 10 μg/mL Poly-L-Ornithine solution. After the confluency was around 90%, the medium was replaced of 0.5 mL fresh DMEM, and 50 μL Reduced Serum Medium (opti-MEM™) containing 2 μL Lipofectamine 2000 and 1 μg pGP-CMV-NES-jRGECO1a plasmids was added into the cells. After incubation for 4 h, the medium was replaced with complete DMEM. The cells were incubated for another 2 days for effective gene transfection. Then, the vial filled with 1 mL Lipo@IR780/L012 solution (10 mg/mL) was fixed over the cells, and the FUS (1.5 MHz, pulse 100 ms FUS on, 900 ms FUS off, 6.2 MPa) was used to treat the solution to generate blue light. The red fluorescence signals were recorded via Leica DMi8 fluorescence microscope. The fluorescence signal was analyzed by Image J software.

### In Vivo Sono-optogenetic Stimulation With Lipo@IR780/L012 Nanoparticles.

Thy1-ChR2-YFP transgenic mice (20-26 g; 4 weeks old; Jackson laboratory) were used in our study. All procedures designed according to the National Institute of Health Guide for the Care and Use of Laboratory Animals, approved by the Institutional Animal Care and Use Committee (IACUC) at the University of Texas at Austin (Protocol ID AUP-2021-00086), and were supported via the Animal Resources Center at University of Texas at Austin. The mice were anesthetized with 2.5% isoflurane, and cut the hair on the head of mice by hair clipper. After that, the mice were fixed in a stereotaxic frame, and placed in a 37° C. heating pad to maintain body temperature. The eyes of the mouse were protected via coating vet ointment. 0.2 mL Lipo@IR780/L012 (10 mg/mL in PBS) was administered through the tail vein.

After injection of Lipo@IR780/L012 nanoparticles, ultrasound gel was placed on the mouse head at first, and then the FUS transducer (1.5 MHz, Image Guided Therapy, France) with 5 mm water balloon was placed on the top of the gel. To achieve effective FUS stimulation, we make sure there are no gaps and bubbles between the transducer and brain, and the gel was totally filled. The stereotaxic coordinates of the motor cortex were anteroposterior (AP) ±1.0 mm, mediolateral (ML) +0.5 mm and dorsoventral (DV) −0.5 mm (Nieuwenhuis et. al., 2021). Then, the isoflurane concentration was adjusted to 0.5%. To guarantee the mouse was in light anesthesia status before conducting the FUS stimulation, we pinched the toes of the mouse to check the response. The FUS stimulation could be started until we observe the body movement when pinching the toes of the mouse. FUS was conducted with repetition frequency of 1 Hz (100 ms on, 900 ms off) and pressure of 6.2 MPa, while the motions of the limbs were recorded through video camera during the FUS stimulation.

Animal behavior tests data processing. Recorded videos of the mice response to ultrasound stimulations were synchronized for quantification of joint angle to during sono-optogenetic stimulation across groups with none, either, or both Lipo@IR780/L012 nanoparticles and ultrasound pulses. Kinematic data were obtained by using DeepLabCut (a markerless pose estimation algorithm based on transfer learning with neural networks for 2D and 3D videos). Images were segmented, down-sampled and extracted into batches of 20 images per video through the use of k-means algorithm as recommended for behavioral study for postures in kinematic data of extremities as opposed to uniform temporal sampling, which is more suitable for global movement of mice within an environment (Nath et. al., 2019). Position of the hip, knee, and joints of the mice were determined by initially labeling 400 images manually for both left and right legs (FIG. 13). 380 of the labeled images (95%) was used for training the neural network model using the ResNet50 framework (commonly used pre-trained network for object recognition) and 20 of the labeled images (5%) were used for verification and evaluation at an iteration of 500,000 epochs. The predicted pose is evaluated with the validation set to determine the maximum error in prediction of the (x,y) coordinates in terms of pixels. Trained model error indicates an average 7.38 pixel and test error evaluated between predicted and labeled pose yielded an error of 9.99 pixel, which yields an error of ±0.603 degrees. Extraction of orientation between marked segments (i.e., hip-to-knee/knee-to-foot) were obtained through calculating the relative segments between the two coordinate points in relation to the horizontal axis of the image. The orientation of segments yielding joint angle was differentiated with respect to time to determine the change in joint angles for quantifying the efficacy of Lipo@IR780/L012 subjected to FUS in mice. This is done by segmenting the data points during which FUS occurs and taking the difference between the maximum (positive) and minimum (negative) peaks and averaged across the groups.

### Example 3

### Sono-Optogenetic Deep Brain Stimulation Via Self-Amplifying Liposomal Nanotransducer

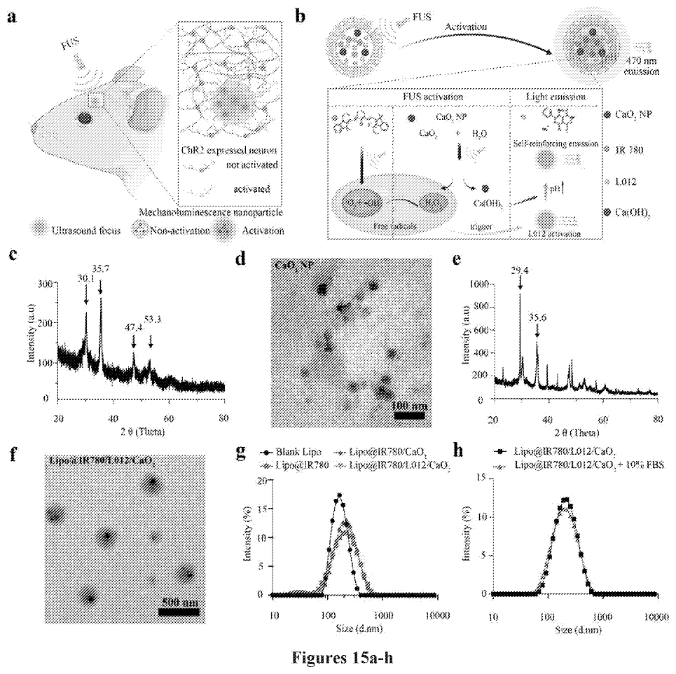

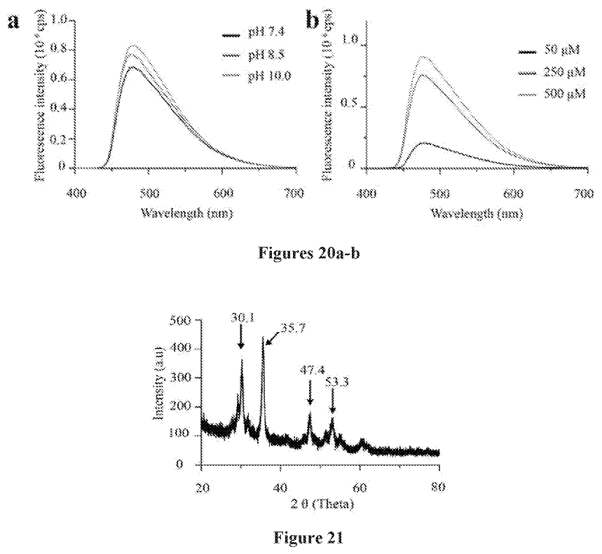

In the above Examples, ultrasound-triggered cascade reactions in Lipo@IR780/L012 liposomes achieved synchronized and stable blue light emission. While these methods were used to stimulate neurons in the brains of mice, the limited light emission intensity was not sufficient to achieve deep brain stimulation. In fact, the local free radical concentration and pH in liposomes both play crucial roles in light emission power. As shown in FIG. 20, the fluorescence intensity of L012 increased by around 30% when the pH changed from 7.4 to 10, and improved around four times when the H₂O₂ concentration was increased from 50 to 500 μM (Daiber et. al., 2004; Yamaguchi et. al., 2010). To achieve improved light emission intensity under ultrasound irradiation, sono-amplifier PEG₂₀₀ coated CaO₂ nanoparticles were first prepared by a calcium chloride (CaCl2)-hydrogen peroxide (H₂O₂) reaction in PEG₂₀₀ solution (Liu et. al., 2017). The specific peaks (2Θ=30.1°, 35.7°, 47.4°, and 53.3°) of CaO₂ are clearly shown in the X-ray diffraction spectrum (FIG. 15c), and the transmission electron microscope (TEM) results determined these nanoparticles have 18±12 nm diameters (FIG. 15d). X-ray diffraction results showed that the CaO₂ nanoparticles have high stability (FIG. 21) in solution but rapidly reacted with water to form H₂O₂ and Ca(OH)₂ (FIG. 15e, 2θ=29.4°, 35.6°) once exposed to ultrasound irradiation. The reaction between CaO₂ nanoparticles and water did not happen without ultrasound since the liposomes membrane and PEG coating layers protected CaO₂ nanoparticles. However, these protective layers are perturbed with ultrasound stimulation so that the water could diffuse in and react with CaO₂ nanoparticles. Our L012, IR780, and CaO₂-loaded liposomes were then prepared using a thin film hydration strategy (Wang et. al., 2021; Jiang et. al., 2021), and their TEM images are shown in FIG. 15f. The dynamic scattering tests determined that the liposome size slightly increased to 178 nm after payload loading (FIG. 15g and Table 3) compared with blank liposomes, and the negative surface zeta potential guaranteed the stability of liposomes in tissue fluid (FIG. 15 h and Table 3). The drug loading capacity of L012 and IR780 in the liposomes was 5.7 wt. % and 6.2 wt. %, respectively, determined via UV-Vis spectroscopy (Table 3). The L012/CaO₂ weight ratio of 1:5 was used in all the following experiments.

TABLE 3
The DLS tests of different liposome solutions.

| | | | | Zeta | DLC of | DLC of |
|---|---|---|---|---|---|---|
| | | Size | | potential | IR780 | L012 |
| entries | Nanoparticles | (d · nm) | PDI | (mv) | (wt %) | (wt %) |
| 1 | Blank Lipo | 166.9 ± 5.9 | 0.174 | −26.0 ± 0.2 | N/A | N/A |
| 2 | Lipo@IR780 | 168.8 ± 0.8 | 0.236 | −10.2 ± 0.1 | 5.70 ± 0.46 | N/A |
| 3 | Lipo@IR780/L012 | 173.3 ± 2.3 | 0.228 | −10.5 ± 0.4 | 5.70 ± 0.20 | 6.0 ± 0.5 |
| 4 | Lipo@IR780/CaO2 | 177.8 ± 1.4 | 0.210 | −11.2 ± 0.2 | 5.83 ± 0.61 | N/A |
| 5 | Lipo@IR780/CaO2/ | 175.9 ± 0.6 | 0.217 | −10.9 ± 0.2 | 5.73 ± 0.47 | 6.17 ± 0.31 |
| | L012 | | | | | |
| 6 | Lipo@IR780/CaO2/ | 177.9 ± 3.1 | 0.233 | N/A | N/A | N/A |
| | L012 + 10% FBS | | | | | |

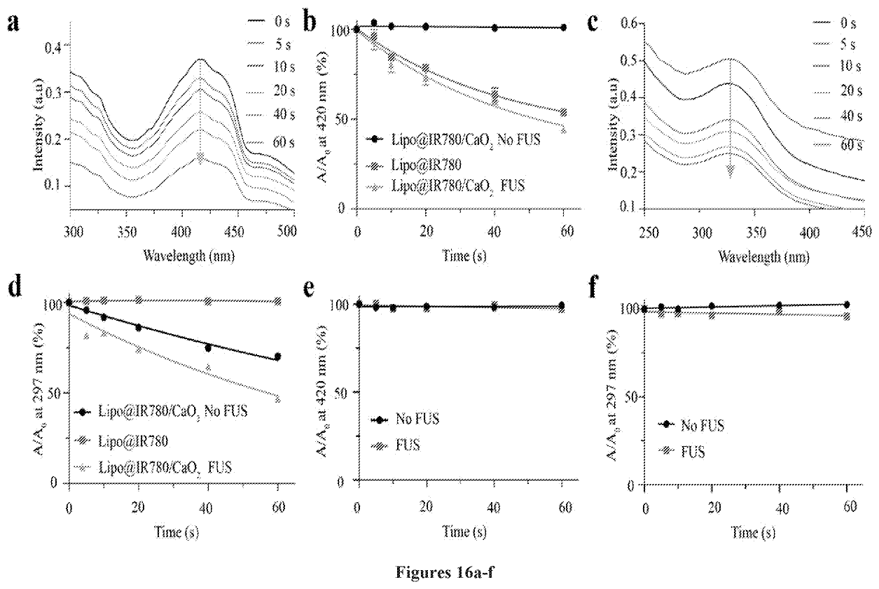

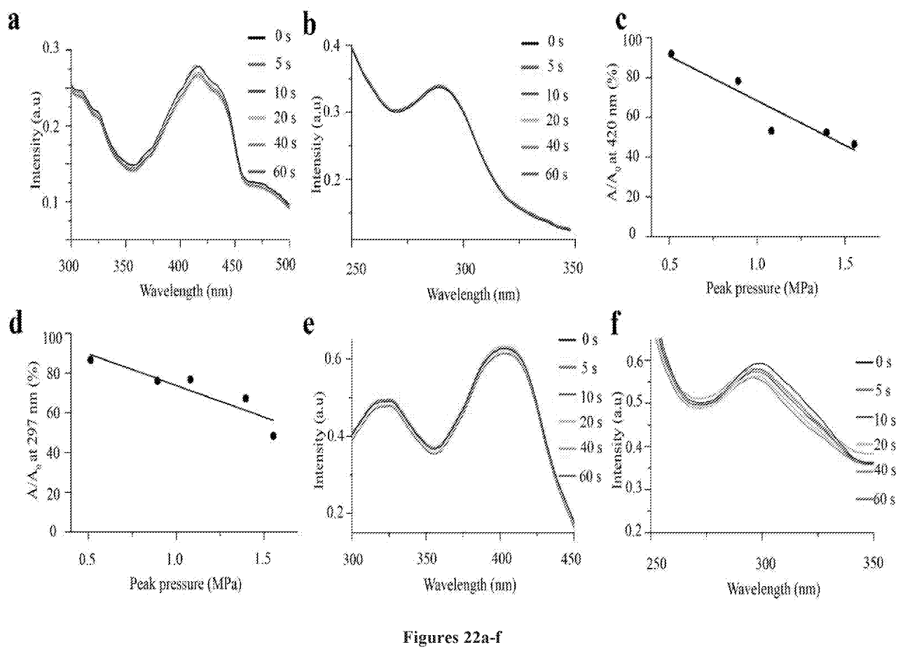

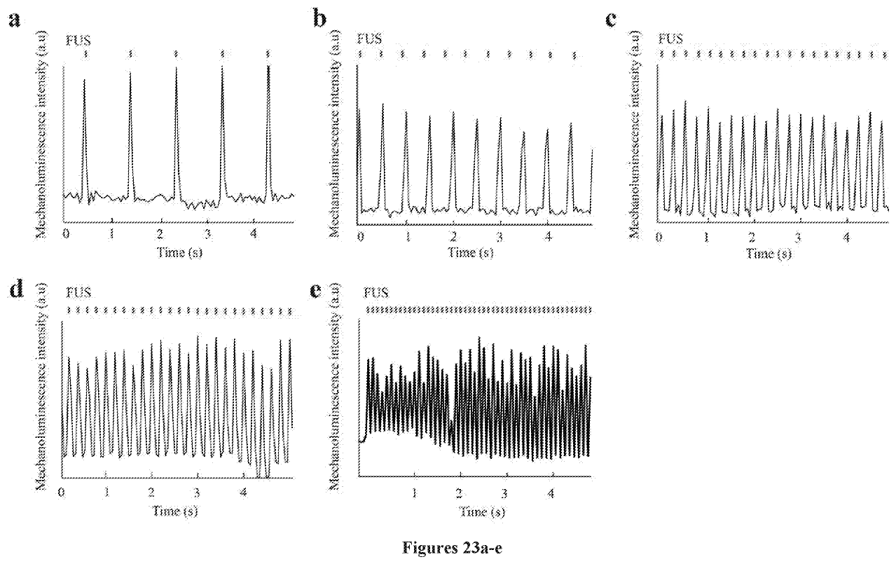

Ultrasound-triggered cascade reactions dominate the spatiotemporal light emission from these Lipo@IR780/L012/CaO₂ liposomes, where the generation of free radicals including singlet oxygen (¹O₂), hydroxyl radical (·OH) and H₂O₂ are first necessary to activate nearby L012 under the irradiation. Thus, we first evaluated the generation of these free radicals in Lipo@IR780/CaO₂ liposomes via different free radical probes. Singlet oxygen (¹O₂) and hydroxyl radical (·OH) were the main free radical species from IR780 under ultrasound irradiation (Wang et. al., 2023). 1,3-diphenylisobenzofuran (DPBF) was used to detect ¹O₂ generation due to its highly specific reactivity (Wang et. al., 2023; Wang et. al., 2018). As shown in FIG. 16a, the characteristic UV-Vis absorption peak of DPBF at 420 nm sharply decreased with the ultrasound irradiation time to form 1,2-dibenzoylbenzene (DBB). Still, no apparent changes were observed when ultrasound irradiation was off (FIG. 22a). The quantification determined that more than 10% DPBF was consumed via Lipo@IR780/CaO₂ liposomes (55.6%) in comparison to Lipo@IR780 liposomes (46.2%) after 60 s FUS irradiation but no changes without FUS irradiation (FIG. 16b). Then, we also evaluated the generation of ·OH and H₂O₂ by measuring the decomposition of salicylic acid (SA). The SA would rapidly react with ·OH and H₂O₂ to form 2,3-dihidroxybenzoic acid and 2,5-dihydroxibenzoic acid (Wang et. al., 2023; Jiménez-Pérez et. al., 2019; Gomes et. al., 2005). As shown in FIG. 16c and FIG. 22b, the characteristic UV-Vis absorption peak of SA at 297 nm dramatically decreased with FUS irradiation time, and no changes were observed without FUS. The quantification showed a decomposition of SA by more than 1.8 times by Lipo@IR780/CaO₂ liposomes (53.2%) in comparison to Lipo@IR780 liposomes (29.7%) after 60 s FUS irradiation. In fact, most of the generated H₂O₂ from CaO₂ nanoparticles could be rapidly detected and consumed via SA, and the remaining degraded to oxygen to improve the ¹O₂ production in the presence of IR780. This reaction model accounts for the significant increase in SA decomposition while DPBF consumption was only slightly increased during FUS stimulation of liposomes incorporating CaO₂. We next evaluated free radical production at different ultrasound powers. As shown in FIG. 22c and FIG. 22d, free radical concentration increased with ultrasound energy, with the Lipo@IR780/CaO₂ liposomes exhibiting higher ultrasound sensitivity and free radical production yield compared with Lipo@IR780. From Lipo@IR780 to Lipo@IR780/CaO₂, the ultrasound power threshold for activation decreased from 0.89 MPa to 0.51 MPa, and free radical yield almost doubled. Finally, L012 is a potent free radical scavenger, and we expected that the free radicals could be temporally quenched via L012 to produce light instead of leaking out to damage nearby cells. Thus, we also detected free radicals in Lipo@IR780/L012/CaO₂ liposomes. The results showed no free radical residues were observed under the FUS irradiation (FIG. 16e-f and FIG. 22e-f).

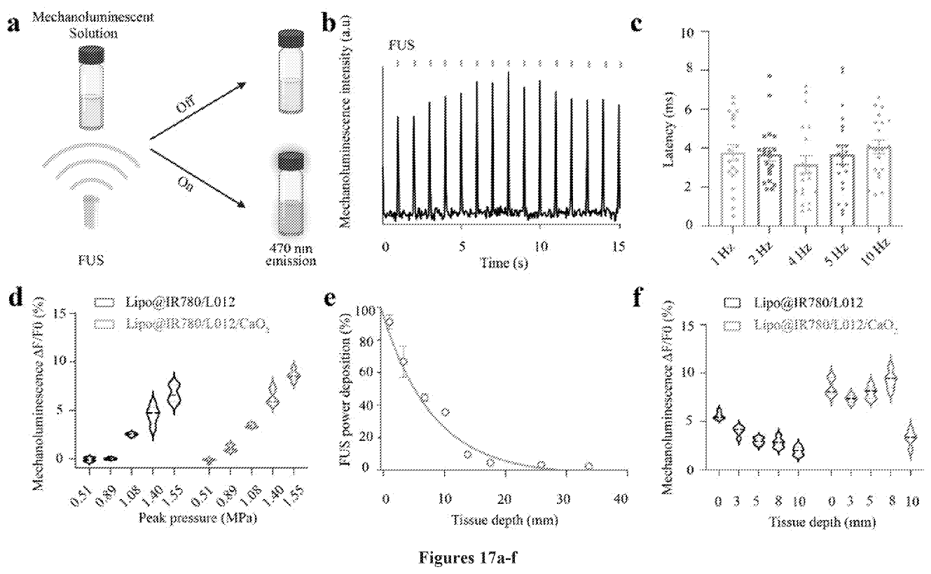

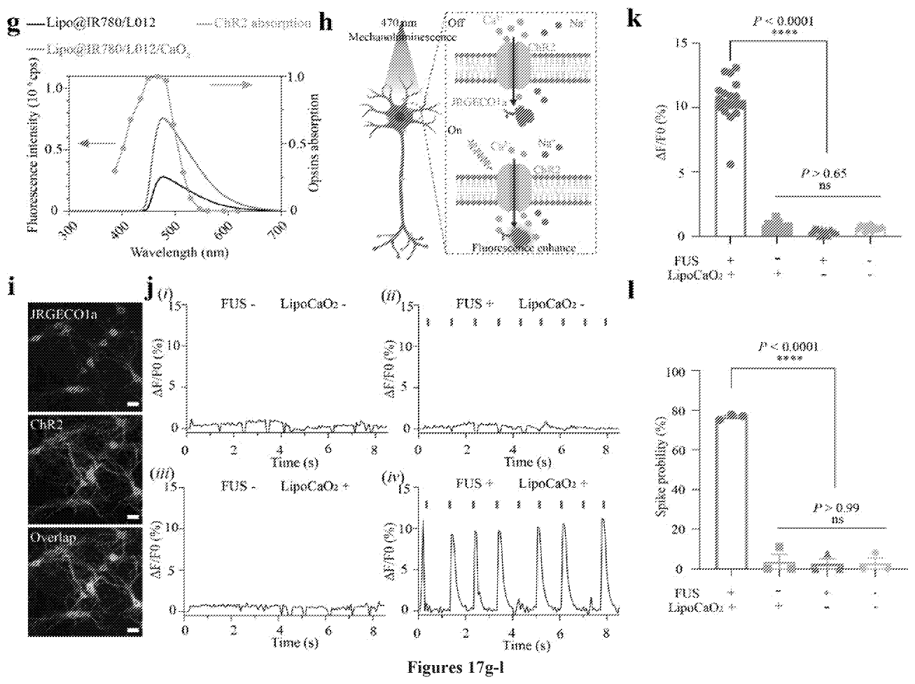

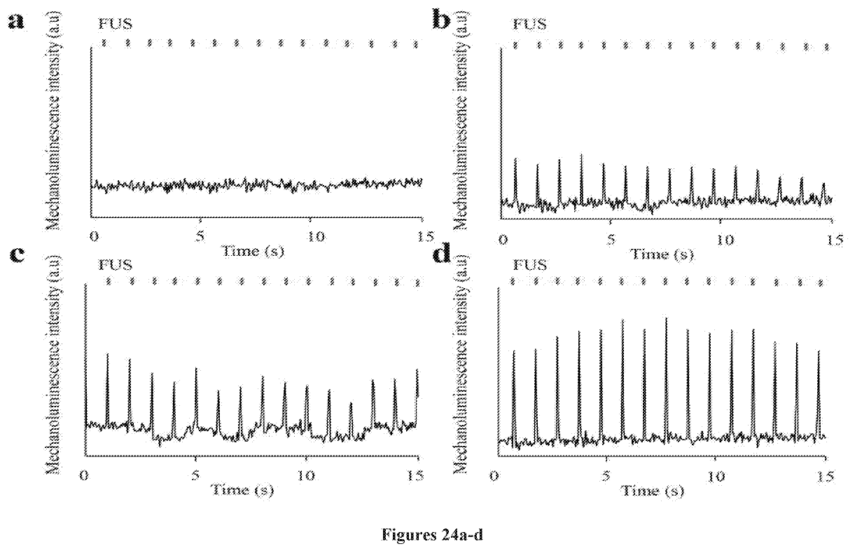

The inventors next investigated the ultrasound-triggered mechanoluminescence performance of Lipo@IR780/L012/CaO2 liposomes (FIG. 17a). The cascade reactions, including free radical generation and quenching, dominated this ultrasound-triggered mechanoluminescence. Free radicals could be generated and quenched for light emission within 5.5 μs and 28 μs, respectively, owing to the high reaction rate constant (4.5×10⁻⁵ M⁻¹s⁻¹, 2.67×10⁻⁸ M⁻¹s⁻¹) (Duco et. al., 2016; Merényi et. al., 1990). Theoretically, light will be generated within 33.5 μs once the liposomes are stimulated via FUS. Time-resolved sono-mechanoluminescence spectra showed that synchronous photons were produced following FUS pulse, where the delay time of light emission was less than 4 ms, even at 10 Hz stimulation (FIGS. 17b, 3c and S4), which is shorter than the time-to-spike latency of approximately 10 ms for ChR2 neuron activation (Boyden et. al., 2005). In addition, we evaluated ultrasound power-dependent light emission, as shown in FIG. 17d and FIG. 24. Lipo@IR780/L012/CaO₂ liposomes exhibited higher photon productivity and ultrasound sensitivity. Next, we investigated ultrasound-triggered photon delivery performance in tissue. Ultrasound energy propagates through tissue as a traveling pressure wave, exponentially attenuating with tissue depth (Stauffer and Paulides, 2014; Vernon and Lewin, 2012). As shown in FIG. 17e, the ultrasound wave of 1.5 MHz could achieve a penetration of 20 mm, with 40% energy delivered even at a tissue depth of 10 mm. The energy transfer efficiency of ultrasound is orders of magnitude higher than both visible and NIR light (Chen et. al., 2018; Chen et. al., 2021; Finlayson et. al., 2022). Moreover, the Lipo@IR780/L012/CaO₂ liposomes exhibited higher ultrasound-triggered photon productivity at a similar tissue depth compared with Lipo@IR780/L012 liposomes, and photon productivity has no evident decrease within a tissue depth of 8 mm (FIG. 17f and FIG. 25). These data demonstrated the potential for achieving remote and wireless photon delivery for minimally invasive brain modulation.

Next, opsin activation in primary neuron cultures under sono-mechanoluminescent irradiation was investigated. The mechanoluminescence spectra of Lipo@IR780/L012/CaO2 liposomes exhibited the maximal emission wavelength at around 470 nm, and the photon yield was about three times higher than Lipo@IR780/L012 liposomes (FIG. 17g). The light emission wavelength mainly overlapped with the channelrhodopsin-2 (ChR2) for optogenetic stimulation (Zhang et. al., 2007). JRGECO1a red calcium indicators were chosen to track neuron activity and minimize spectral overlap (FIG. 17h) (Vogt, 2016; Inoue et. al., 2019). Neurons transduced with AAV9-hSyn::ChR2-EYFP and AAV9-hSyn::NES-JRGECO1a.WPRE.SV40 (FIG. 17i) exhibited synchronized firing after the irradiation in the presence of ultrasound (FUS+) and Lipo@IR780/L012/CaO₂ liposomes (LipoCaO₂+) with around 80% spike probability, but no evident increase of calcium fluorescence was observed in all other control groups (FIG. 17j-l).

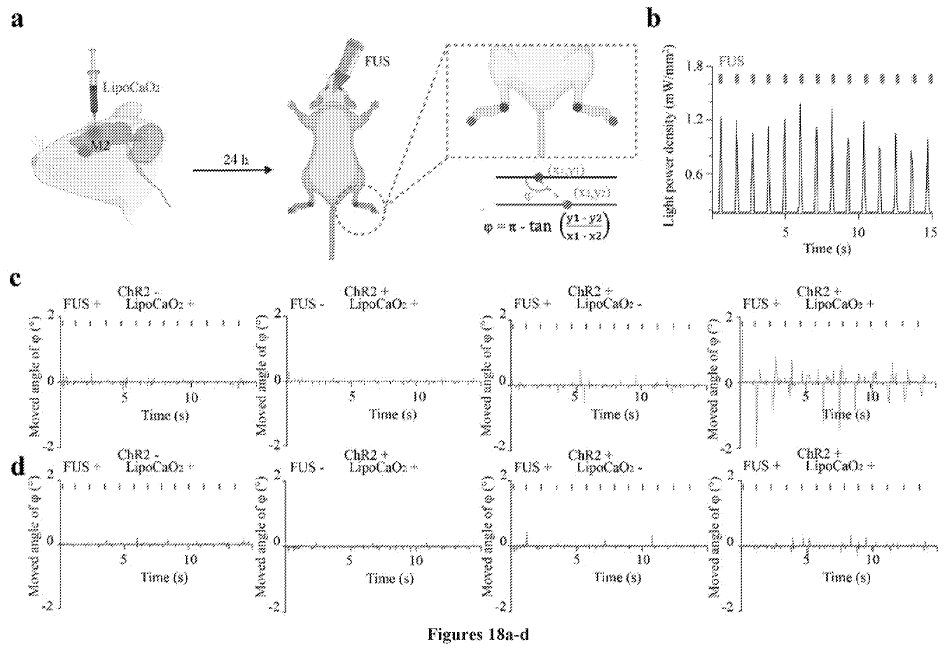

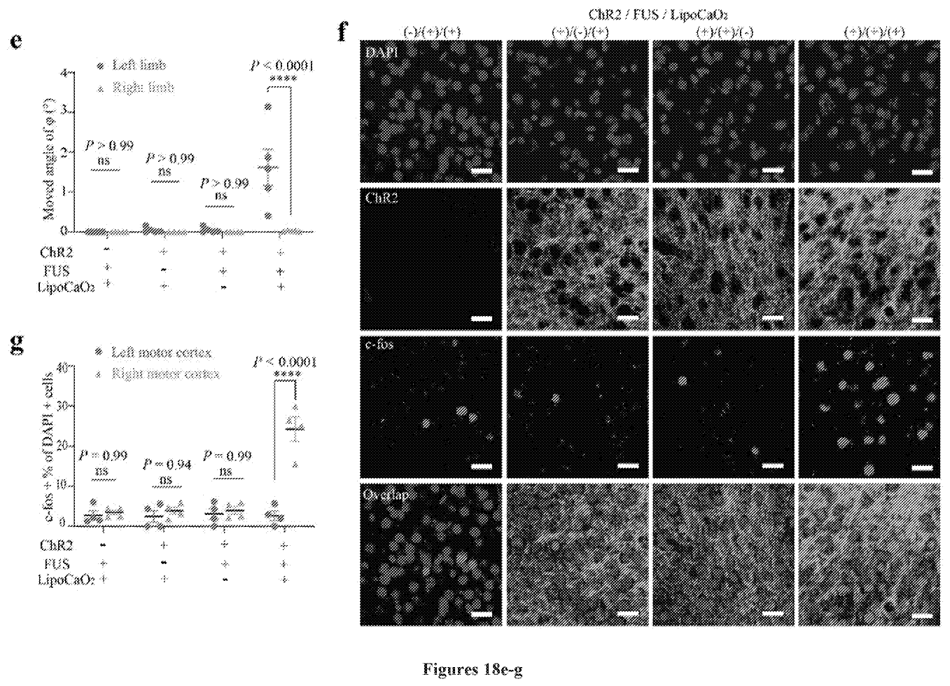

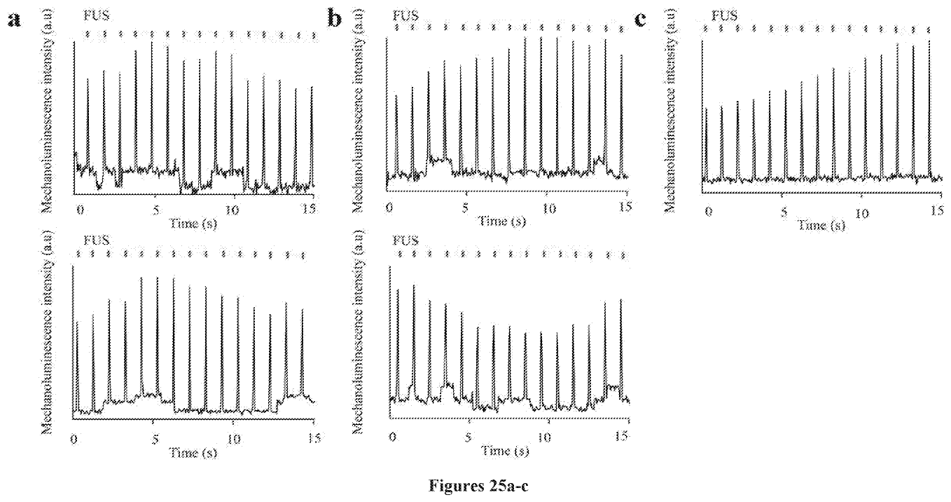

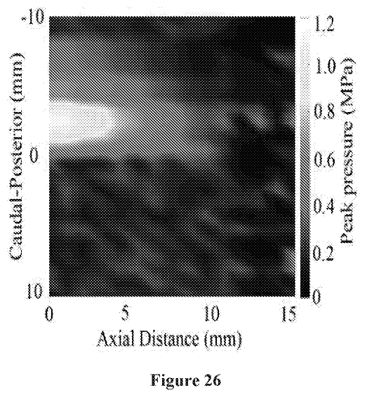

The inventors next tested sono-optogenetic neural activation in the mouse secondary motor cortex (M2) where optogenetic activation is expected to modulate limb motion. The Lipo@IR780/L012/CaO₂ liposomes were unilaterally injected into the right M2 of Thy1-ChR2-YFP transgenic mice. After 24 h, FUS was applied to the M2 region of the mouse brain (FIG. 18a). The normalized ultrasound energy heat map at the mouse motor cortex showed that around 1.15 MPa peak pressure was delivered to the M2 region when 1.55 MPa primary ultrasound energy was used (FIG. 26). The high energy transfer efficiency ensured that these liposomes could be effectively activated. As shown in FIG. 18b, the synchronous blue light with power intensity 1.21 mW/mm² was generated under the FUS stimulation, which should be sufficient to achieve more than 60% wild-type ChR2 spike probability (Chen et. al., 2021; Klapoetke et. al., 2014). Since the motor cortex is responsible for higher-order control of movement (Yang and Kwan, 2121), we tested in vivo sono-optogenetic stimulation in anesthetized subjects by video tracking of contralateral and ipsilateral limbs (FIG. 18a). As shown in FIG. 18c-d, DeepLabCut analysis determined that contralateral left limb motion was observed in Thy1-ChR2-YFP transgenic mice with FUS stimulation with liposome injection, while no ipsilateral limbs motion was observed. Limb motion was not activated in the absence of FUS stimulation or liposome injection, or in wild-type mice. We anticipate seeing some limb motion in wild-type mice with FUS stimulation of endogenous mechanosensitive ion channels and anticipate the absence such non-specific activity is a result of anesthesia (Kubanek et. al., 2018; Sato et. al., 2018; Blackmore et. al., 2019; Yu et. al., 2121). We next evaluated neuron activation in post-hoc tissue samples via expression of immediate early gene marker c-Fos. A dramatic increase in c-Fos signals was selectively observed in the right M2 region in subjects receiving both FUS stimulation and liposome injection (FIG. 18f-g and FIG. 27). These results suggested that sono-optogenetics with our novel liposome nanotransducers is sufficient to achieve effective, remote, and minimally invasive photon delivery in the motor cortex for neuron activation.

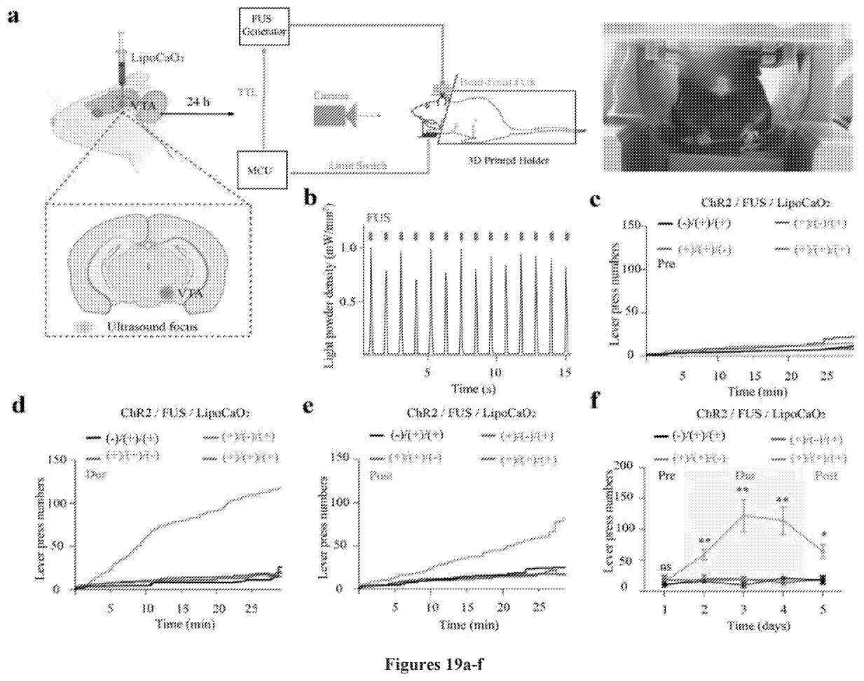

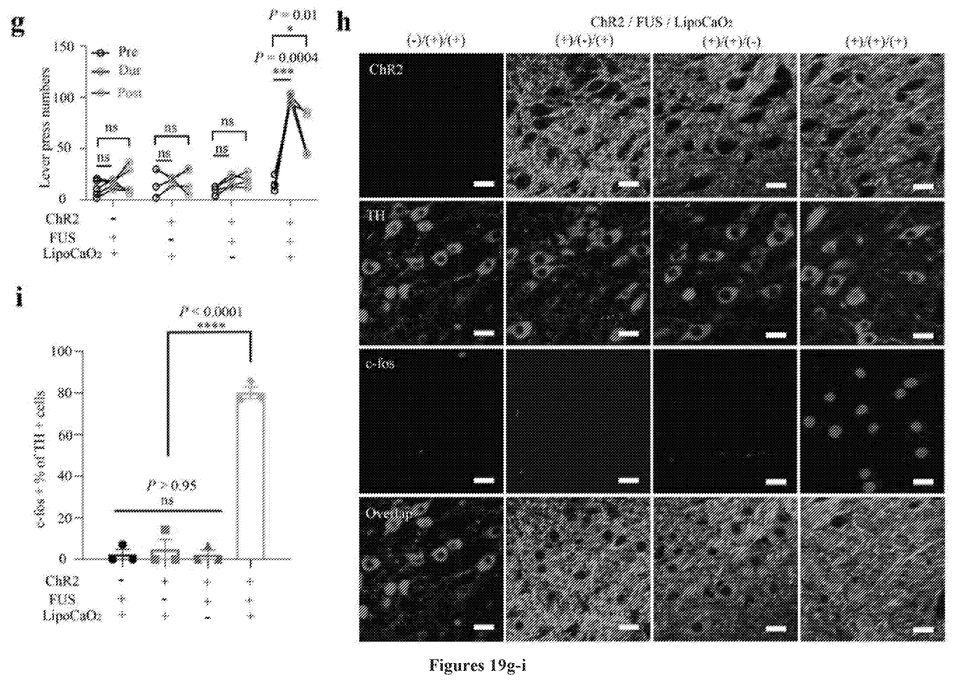

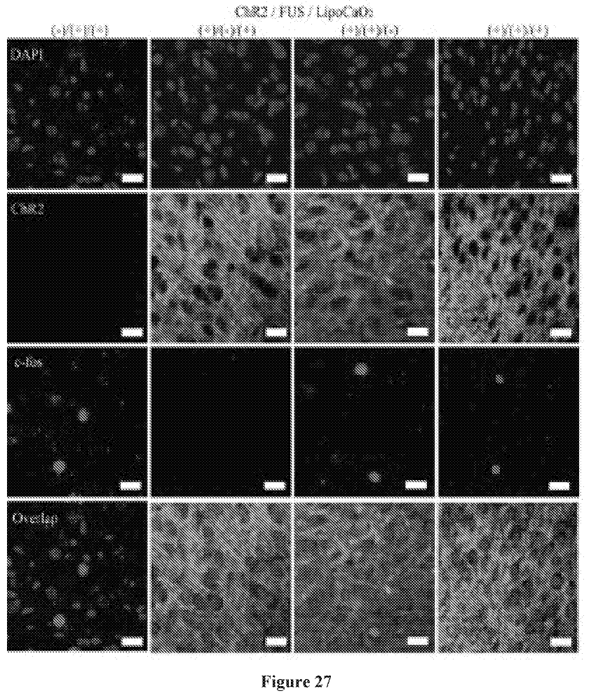

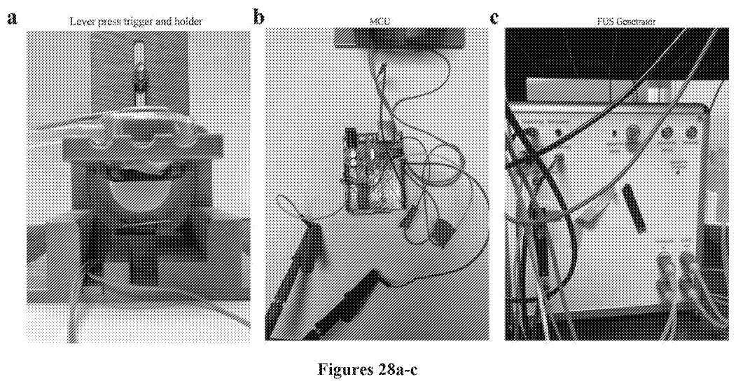

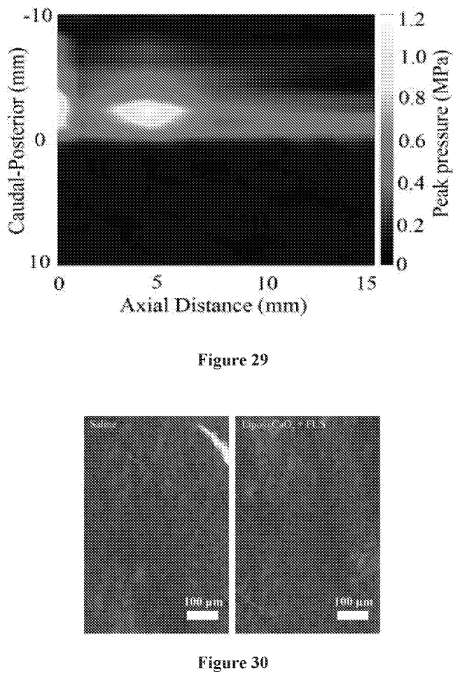

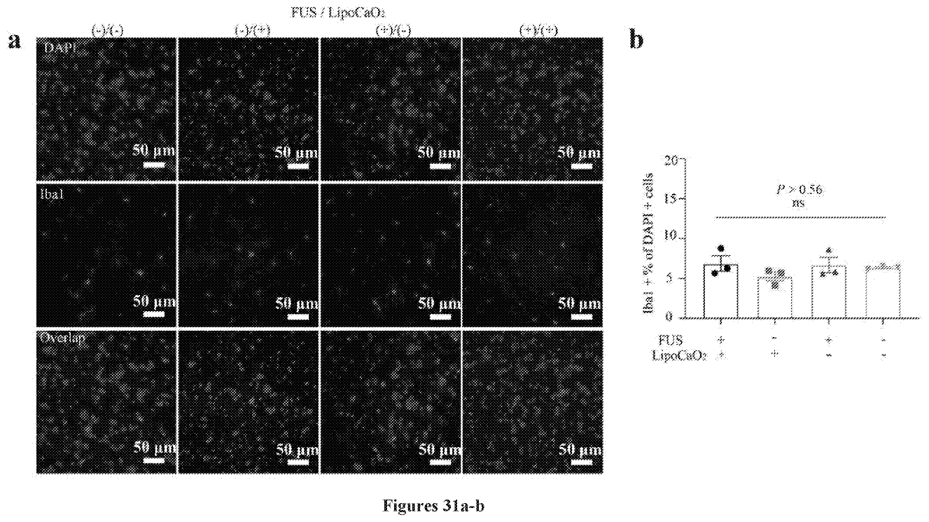

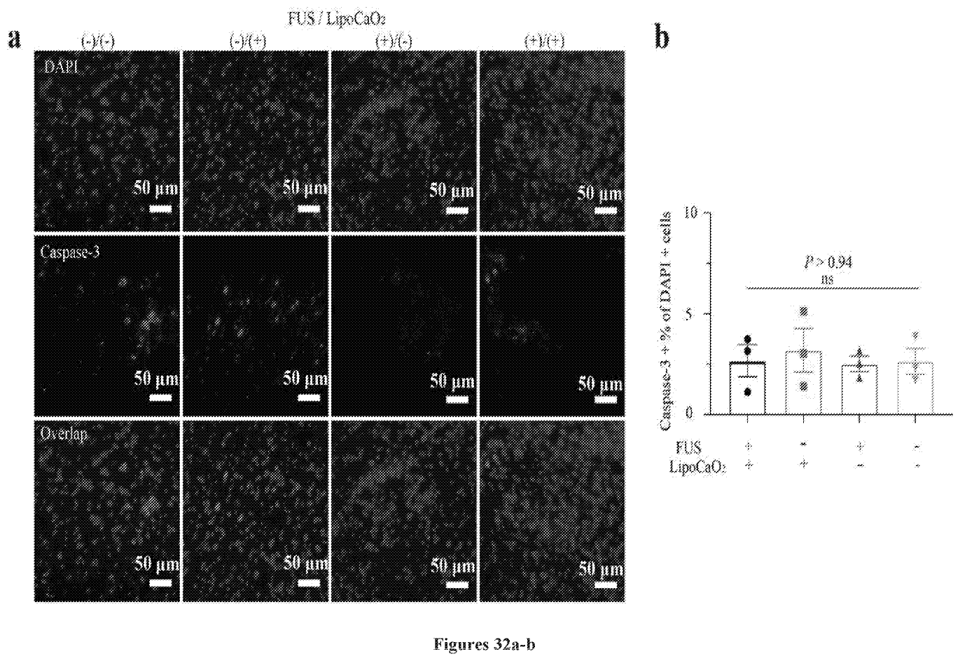

Finally, we investigated ultrasound-triggered deep photon delivery in the mouse Ventral Tegmental Area (VTA). The VTA is well known for regulating both motor behavior and reward learning via dopaminergic projections (Cai and Tong, 2022; Rao et. al., 2019). We chose to assay the function of our sono-optogenetic approach in a head-fixed, lever pressing paradigm, which allows the animal to activate the ultrasound trigger (FIG. 19a and FIG. 28). Before behavioral evaluation, we first assayed ultrasound-driven in vivo light emission at the VTA (1.5 MHz, 1.55 MPa, pulse 100 ms on, 900 ms off). Due to the high energy transfer efficiency of ultrasound in brain tissue, around 0.97 MPa of ultrasound energy from 1.55 MPa of primary ultrasound source was measured at the VTA, which is sufficient to activate liposomal light emission (FIG. 29). The time-resolved light emission spectra showed that the temporal blue light of 1.0 mW/mm² power density was detected under ultrasound stimulation (FIG. 19b), which is sufficient to activate ChR2-expressing neurons. Liposomes were unilaterally injected into the mouse VTA region and an ultrasound stimulation metal ring was affixed to the skull. 24 h after surgery, the mouse was placed in the 3D-printed holder allowing the animal to reach the ultrasound-triggering lever with the front limbs. Upon pressing the lever, the FUS transducer was programmed to generate one pulse (100 ms on, 1.5 MHz, 1.55 MPa). To systematically investigate the reward-seeking behaviors under sono-optogenetics, we tracked the mouse press number over 5 days, including prestimulus (Pre, the FUS pulse is always off) at day 1, during stimulation (Dur, a FUS pulse was generated once the mouse presses the lever) at day 2 to day 4, and poststimulus (Post, the FUS pulse is always off) at day 5. Mice were not observed to have an innate preference for lever pressing (FIG. 19c). However, the Thy1-ChR2-YFP transgenic mice administered both liposomes and FUS stimulation exhibited rapidly increased lever pressing rates with FUS (Dur), and this preference was preserved across trial days, as observed with continued lever pressing without FUS stimulation (Post) (FIG. 19d-g). We again evaluated expression of c-Fos (FIG. 19h-i) and observed a significant increase in c-Fos signal in the Thy1-ChR2/FUS/liposome condition, including in TH+ DA neurons. These results showcase the ability of our sono-optogenetic system to effectively deliver photons to the VTA, activate DA neurons, and achieve remote and minimally invasive modulation of reward learning behaviors. Finally, we investigated the in vivo biosafety and biocompatibility of this system. Seven days after sono-optogenetic stimulation, the brain sections stained with hematoxylin and eosin (H&E) showed that liposomes did not result in notable cell toxicity (FIG. 30). We also noted no difference across samples in expression of glial activation (Iba1; FIG. 31) or neuron apoptosis (Caspase-3; Figure S13).

### Example 4

### Experimental Methods

The following materials and methods were used in the experiments performed in Example 3.

Chemicals: Soybean phosphatidylcholine (DSPC), 1,2-dipalmitoyl-sn-glycero-3-phosphocholine (DPPC) and L012 sodium salt (L012), were purchased from MedChemExpress. cholesterol, 1,2-distearoyl-sn-glycero-3-phosphoethanolamine-N-[amino(polyethylene glycol)-2000) (DSPE-PEG₂₀₀₀), IR-780 iodide (IR-780), 1,3-Diphenylisobenzofuran (DPBF), salicylic acid (SA), sodium hydroxide (NaOH), 30 wt. % hydrogen peroxide aqueous solution (H₂O₂), calcium chloride (CaCl₂), polyethylene glycol 200 (PEG₂₀₀) were purchased from Sigma-Aldrich.

Characterization: The morphology of nanoparticles were investigated via FEI TECNAI G2 F20 X-TWIN Transmission Electron Microscope (TEM). The hydrodynamic size and surface zeta potential of nanoparticles were measured via dynamic light scattering (DLS, Zetasizer Nano-ZS from Malvern Instruments). The UV-vis spectrum was measured via Eppendorf Biospectrometer. The sono-mechanoluminescence spectrum was measured via Fluorolog3 Fluorimeter. The focused ultrasound (FUS) generator and transducer was ordered from Image Guided Therapy. The fluorescence images and videos were recorded by Leica DMi8 fluorescence microscope. Confocal images were obtained from the Zeiss LSM 710 confocal laser scanning microscope. The powder X-ray diffraction patterns were collected by a Panalytical X'Pert powder diffractometer equipped with a Cu sealed tube (λ=1.54184 Å) at 40 kV and 40 mA over the 2θ range of 5-40°.

The fluorescence spectrum tests of L012 at different pH and H₂O₂ concentration. the quantum yield of the L012 solution at different Ph was investigated. 1 mL L012 solution (0.5 mg/mL) with pH 7.4, 8.5, and 10.0 was mixed with H₂O₂, where the final concentration of H₂O₂ was 250 μM. The fluorescence spectrum was recorded via Fluorolog3 Fluorometer. In addition, we also investigated the quantum yield of the L012 solution with different H₂O₂ concentrations at pH 7.4. 1 mL L012 solution (0.5 mg/mL) with different H₂O₂ concentrations (50, 250, and 500 μM) at pH 7.4 was prepared at first, and the fluorescence spectrum was also recorded via Fluorolog3 Fluorometer.

Preparation of CaO₂ nanoparticles. The CaO₂ nanoparticles were prepared via CaCl₂-H₂O₂ reaction following the previous method (Liu et. al., 2017). In brief, 1 mL CaCl₂ solution (0.1 g/mL in distilled water) and 0.5 mL ammonia solution (1M) were added into 80 mL PEG200 with a stirring speed of 1000 rpm in the flask. After stirring for 30 min, the CaCl₂ solution was totally dispersed into the PEG200 solution, and 0.5 mL 30 wt. % H₂O₂ solution was dropwise added to the mixture within 10 min. Then, the mixture was stirred for another 6 h at room temperature to obtain a colorless solution and then transfer to the sonication bath. 1 M NaOH solution was dropwise added into the mixture to adjust the pH value to 11.5. In this process, we could observe that the clear solution became slightly white. The mixture was centrifuged at speed of 15000 rpm/min, and the precipitate was washed with 0.1 M NaOH solution, distilled water, and ethanol to remove excessive PEG₂₀₀. The PEG₂₀₀ coated CaO₂ nanoparticles were stored in ethanol at 4° C. for future use. After removing the solution, the CaO₂ nanoparticles powder was used to detect the crystal structure through X-ray diffraction analysis. 0.1 mg/mL CaO₂ nanoparticles solution was used to prepare TEM samples for morphology tests.

Preparation of drug loaded lipids nanoparticles (liposomes). To prepare the liposomes, thin film hydration strategy was used (Wang et. al., 2021; Jiang et. al., 2021). Briefly, 16 mg DSPC, 0.8 mg DPPC, 2.4 mg cholesterol, 0.6 mg DSPE-PEG2000 1.2 mg IR780, and 0.75 mL CaO₂ ethanol solution (10 mg/mL) was dissolved in 4 mL chloroform and 1 mL methanol mixture. The solution was removed after rotary evaporation, and a thin lipid film was obtained. 3 mL L012 solution (0.5 mg/mL) was added into the lipid film, and treated in a sonication bath for 1 min at 60° C. The mixture solution was extruded via a 0.45 um filter to obtain uniform lipid suspension. Then the suspension was centrifuged at 12000 rpm/min for 20 min, the precipitate was resuspended via 2 mL distilled water. 0.5 mg/mL liposomes solution was used to do the TEM and DLS tests. The calcium concentration was measured via inductively coupled plasma-atomic emission spectrometry. In addition, the obtained liposomes solution was dried via freezing dry to obtain powder and stored with foil coverage at 4° C. for future use.

The generation of ¹O₂ under the FUS stimulation in liposomes. The IR780 loaded liposomes (Lipo@IR780), IR780/CaO₂ loaded liposomes (Lipo@IR780/CaO₂) and IR780/CaO₂/L012 laded liposomes (Lipo@IR780/CaO₂/L012) were used to evaluate the free radicals generation. 1 mL liposomes solution (1 mg/mL) was mixed with 30 μL 1 mg/mL DPBF methanol solution in a dark room. Then, the mixture was continuously irradiated with or without FUS (1.5 MHz, 1.55 MPa), and 10 μL of the solution was extracted at different time points for UV-vis spectrum tests. The characteristic absorption peak of DPBF at 420 nm was real-time detected to quantify the generation of ¹O₂. In addition, we also measured the UV-vis spectrum of the mixture after treating 60 s under different FUS power (from 0 to 1.55 MPa), where the ultrasound power was measured via hydrophone (Onda Corporation, HGL-0200).

The generation of ·OH and H₂O₂ under the FUS stimulation in liposomes. Similar to ¹O₂ detection, 1 mL liposomes solution (1 mg/mL) was mixed with 50 μL 1 mg/mL SA methanol solution in a dark room. SA could effectively react with ·OH and H₂O₂, we therefore could track the consumption of SA to quantify the generation of ·OH and H₂O₂. The UV-vis characteristic absorption peak of SA is at 297 nm. After irradiation with or without FUS (1.5 MHz, 1.55 MPa), 10 μL of the liposomes mixture was extracted at different time points for UV-vis spectrum tests. Moreover, we also tested the UV-vis absorption of the liposomes mixture after treating for 60 s under different FUS power (from 0 to 1.55 MPa).

FUS triggered blue light emission from Lipo@IR780/L012 and Lipo@IR780/CaO₂/L012 liposomes. 1 mL liposomes solution (5 mg/mL) was added into a 2 mL glass vial, and fixed on the top with a FUS transducer (1.5 MHz, Image Guided Therapy). The gap between the glass vial and transducer was filled with ultrasound gel, and a video camera (CS165MU1/M-Zelux® 1.6 MP Monochrome CMOS Camera, Thorlabs) was placed in front of the glass vial to record the light emission in the dark room. We first tested the light emission at a repetition frequency of 1 Hz (pulse 50 ms on 950 ms off) with different ultrasound power, from 0 to 1.55 MPa. Then, we also evaluated the light emission at different irradiation frequencies at 2 Hz (50 ms FUS on, 450 ms FUS off), 4 Hz (50 ms FUS on, 200 ms FUS off), and 5 Hz (50 ms FUS on, 150 ms FUS off) and 10 Hz (50 ms FUS on, 50 ms FUS off) at peak pressure 1.55 MPa. All the parameters were fixed to record the video, and the data were analyzed by ImageJ software. To investigate the delay time between the FUS stimulation and light emission from liposomes, we simultaneously recorded the FUS pulse LED indicator light and mechanoluminescence light, and the time gap between these two emissions is the latency.

The evaluation of FUS power transmission efficiency in tissue. In this test, we use pork skin to mimic the normal tissue. Different depth pork skin was placed on the top of the transducer with ultrasound gel filling. Then, the ultrasound hydrophone (Onda Corporation, HGL-0200) was tightly placed behind the pork skin with ultrasound gel filling. The ultrasound power was fixed, and the peak pressure behind the different depths of pork skin was recorded via a hydrophone, thus calculating the ultrasound transmission efficiency.

The light emission from Lipo@IR780/L012 and Lipo@IR780/CaO₂/L012 liposomes at deep tissue. We also recorded the light emission from liposomes under the FUS irradiation at different depths of pork skin. Similarly, the pork skin with different depths was placed on the top of the transducer, and the liposomes solution loaded in 2 mL glass vial was placed behind the pork skin with ultrasound gel filling. FUS (1.55 MPa, pulse 50 ms on 950 ms off) was used to treat the solution, and the light emission was recorded via CS165MU1/M-Zelux® 1.6 MP Monochrome CMOS Camera.

The sono-mechanoluminescence spectrum tests of Lipo@IR780/L012 and Lipo@IR780/CaO₂/L012 liposomes. 2 mL liposomes solution (the equivalent concentration of L012 is 1 mg/mL) was added into a plastic cuvette and insert the fluorescence spectrometer (Fluorolog3 Fluorometer), the FUS transducer was placed on the side of the plastic cuvette, and the gap was filled with ultrasound gel. The liposomes solution was treated at peak pressure 1.55 MPa with pulse 3 s on 5 s off, and the sono-mechanoluminescence was recorded.ChR2 and ChiReff absorption spectra were referred from previous work (Zhang et. al., 2007; Hochbaum et. al., 2014), and extracted via GetDataGraphDigitizer software.

In vitro sono-optogenetics tests. We evaluated the sono-mechanoluminescence triggered firing ChR2 expressing primary neurons. Primary cortical neurons were used in our tests. Briefly, the pregnant C57BL/6 mouse (20-26 g; 8 weeks old; Jackson Laboratory) was sacrificed when the pups were 15.5 days old, and these pups' brains were used to prepare the primary cortical neurons. The 24 well plates were coated with poly-1-ornithine (0.2 mg/mL) at 37° C. for 2 h, and washed with PBS several times to remove excessive poly-1-ornithine, then warmed at 37° C. cell incubator before use. The dissociated cortical neurons were plated into the plate with suitable cell density, and cultured in neurobasal medium with 10% B27, glutamine, penicillin, and streptomycin. After incubating for 2 days at 37° C. under 7% CO₂, the glial inhibitor 5-fluoro-2′-deoxyuridine (0.1 mM) was added. After 4 days of incubation, 0.5 μL pAAV-hSyn-hChR2 (H134R)-EYFP and 0.5 μL pAAV.Syn.NES-JRGECO1a.WPRE.SV40 were added to infect the neurons. After another 7 days of incubation, the ChR2 opsins and JRGECO1a calcium indicator were successfully expressed in the neurons for calcium imaging tests. Similar to spiking HEK cells calcium imaging tests, the vail filling with 2 mL Lipo@IR780/CaO₂/L012 liposomes solution with 1 mg/mL equivalent concentration of L012 was fixed over the cells, and the FUS irradiation (1.55MPa, pulse 100 ms on 900 ms off) was given to activate the system for light generation. The jRGECO1a red fluorescence signals were collected and recorded via Leica DMi8 fluorescence microscope. The spiking fluorescence data were analyzed by ImageJ software.

Ultrasound Heatmap determination in mice brain. CB57BL/6 wild type mice (20-26 g; 8 weeks old, Jackson Laboratory) were sacrificed via intraperitoneal injection of ketamine. The head was then removed surgically with the skin intact and stored in 1% paraformaldehyde (PFA) for 48 hours at 4° C. Upon usage, the head was removed from the PFA solution and cleansed with distilled water for 2 minutes. Increments of 1 mm from posterior to anterior measurements of the width (Medial-Lateral) and depth (Cranial-Caudal) were performed prior to dissection and heatmap measurements. A hydrophone (ONDA HGL200, Onda Corporations) was mounted to a custom-made 3-axis system and connected to a preamplifier towards an oscilloscope for measurement. During measurement, the hydrophone was placed in contact to the caudal section of the head while the commercial transducer (25 mm OD@1.5 MHz FUS, Image Guided Therapy System) was in contact with the cranial section. The placement of the transducer differs for motor cortex and VTA stimulation and was adjusted manually in accordance with the behavioral experimental procedures. For both contacts, ultrasound gel was applied to ensure maximum contact and FUS transmission (Ultrasound Transmission Gel 100, Aquasonic) To begin measuring the ultrasound heatmap, surgical scissors were used to remove sections of the skull and brain incrementally from the caudal sections. Per each removal, a reiteration of measurement in dimension was performed in conjunction with the placement of the hydrophone The hydrophone moved from anterior to posterior in increments of 1 mm and FUS pulse with peak pressure 1.55 MPa was sonicated. The measured voltage signal from the hydrophone was then converted to pressure via a calibration curve provided by the datasheet from Onda Corporations. A 10 mm by 15 mm acoustic distribution of the FUS were obtained in the sagittal plane of the mouse head with skin and skull intact. The data was then processed via linear interpolation in MATLAB for higher resolution representation of the acoustic field shown in FIGS. 26 and S29.

Vertebrate animal subjects. Thyl-ChR2-YFP transgenic mice (20-26 g; 4 weeks old; Jackson laboratory) and C57BL/6 wild type mice (20-26 g; 8 weeks old; Jackson Laboratory) were used in our study. All procedures were designed according to the National Institute of Health Guide for the Care and Use of Laboratory Animals, approved by the Institutional Animal Care and Use Committee at the University of Texas at Austin, and were supported via the Animal Resources Center at the University of Texas at Austin.

In vivo Stereotaxic injection of liposomes at the motor cortex. Thy1-ChR2-YFP transgenic mice and wild type mice were used. Before the surgery, all tools were autoclaved. The mice were anesthetized by an isoflurane anesthesia machine (Vaporizer Sales & Service Inc) with 2.5% concentration and the head was fixed in a stereotaxic frame (Kopf Stereotaxic Instruments). The head hair was shaved and the head skin was cleaned three times with isopropanol and iodophor, respectively. Then, meloxicam (5 mg/kg) and Ethiqa (3.25 mg/kg) were subcutaneously injected before surgery, and the eyes were protected from the ophthalmic ointment. The isoflurane concentration decreased to 1.5% when the surgery was conducted. 2 μL liposomes solution (100 mg/mL dissolved in sterilized PBS) was unilaterally injected into the right motor cortex, with the coordinates relative to bregma: anteroposterior (AP) +1.0 mm, mediolateral (ML) +0.50 mm, and dorsoventral (DV) −0.5 mm.

In vivo sono-optogenetic tests for motor cortex modulation. After 24 h recovery, the mice were used to do the motor cortex behavior tests. The mice were anesthetized with 2.5% isoflurane, and the head was fixed in a stereotaxic frame, where the 37° C. heating pad was placed under the mouse body to maintain temperature. The eyes were protected from the ophthalmic ointment. Then, the FUS transducer was placed on the top of mice head with ultrasound gel filling to cover the whole motor cortex region, the transducer coordinates relative to bregma: anteroposterior (AP) 0.0 mm, mediolateral (ML) +0.50 mm, and dorsoventral (DV) −0.5 mm. After that, the isoflurane concentration decreased to 0.5% to make sure the mice were in light anesthesia before stimulation. FUS pulse (1.55 MPa, 1.5 MHz, 100 ms on 900 ms off) was given to control the limb motion, and the motions were recorded via video camera. The mice limb motion data were analyzed via DeepLabCut according to the previous method (Wang et. al., 2023).

In vivo Stereotaxic injection of liposomes at VTA for lever press tests. Thy 1-ChR2-YFP transgenic mice and wild type mice were used. Before the surgery, all tools were autoclaved. The mice were anesthetized by an isoflurane anesthesia machine (Vaporizer Sales & Service Inc) with 2.5% concentration and the head was fixed in a stereotaxic frame (Kopf Stereotaxic Instruments). The head hair was shaved and the head skin was cleaned three times with isopropanol and iodophor, respectively. Then, meloxicam (5 mg/kg) and Ethiqa (3.25 mg/kg) were subcutaneously injected before surgery, and the eyes were protected from the ophthalmic ointment. The isoflurane concentration decreased to 1.5% when the surgery is conducted. 2 μL liposomes solution (100 mg/mL dissolved in sterilized PBS) was unilaterally injected into the right motor cortex, with the coordinates relative to bregma: anteroposterior (AP) +3.08 mm, mediolateral (ML) +0.40 mm, and dorsoventral (DV) −5.0 mm. After that, the head plate (Model 13, Neurotar) was mounted in the head for lever press tests.

After 24 h recovery, the mice were placed in the 3D printed holder, and the front limbs were placed on the trigger. The FUS transducer was placed on the mouse head with hydrogel filling, and the coordinates relative to bregma: anteroposterior (AP) ±0 mm, mediolateral (ML) +0.40 mm, and dorsoventral (DV) −5.0 mm. The FUS pulse (1.55 MPa, 1.5 MHz, 100 ms on) will be given once the mice press trigger. In the lever press behavior tests, we separated three sessions, including prestimulus (Pre), during (Dur), and poststimulus (Post) session. In the prestimulus session (day 1, 30 min), the mice were placed to obtain the level press number baseline, where the FUS generator was off, and no FUS pulse was given when the trigger was pressed, and we only recorded the level press number in 30 min. In the Dur session (day 2-day 4, 30 min each test), the FUS generator is on, and the FUS pulse (1.55 MPa, 1.5 MHz, 100 ms on) was given when the trigger was pressed, the press number was recorded within 30 min. In Post session (day 5, 30 min), no FUS pulse was given when the trigger was pressed, and we only recorded the level press number in 30 min to study the addiction behaviors.

In vivo light intensity power tests in the motor cortex and VTA under the FUS stimulation. We also determined the in vivo light intensity according to the previous method. The wild type mice were anesthetized by an isoflurane anesthesia machine (Vaporizer Sales & Service Inc) with 2.5% concentration and the head was fixed in a stereotaxic frame (Kopf Stereotaxic Instruments). The head hair was shaved and the head skin was cleaned three times with isopropanol and iodophor, respectively. Then, meloxicam (5 mg/kg) and Ethiqa (3.25 mg/kg) were subcutaneously injected before surgery, and the eyes were protected from the ophthalmic ointment. Then two optical fibers (CFML12L05, Thorlabs) were inserted into the motor cortex (anteroposterior (AP) +1.0 mm, mediolateral (ML) +0.5 mm, dorsoventral (DV) −0.5 mm) or VTA (anteroposterior (AP) +3.08 mm, mediolateral (ML) +0.4 mm, dorsoventral (DV) −5.0 mm). One of the fibers was connected to a blue LED (LEDD1B-T-Cube LED Driver, Thorlabs), and another one was connected to FLIR Blackfly S BFS-U3-51S5M-C Camera. The photons density was recorded via the camera, and we recorded the photons density at different LED power densities to obtain a light density calibration curve at first.

Then, in order to test the light power density from liposomes at the motor cortex or VTA, the liposomes were firstly injected into the motor cortex (anteroposterior (AP) +1.0 mm, mediolateral (ML) +0.5 mm, dorsoventral (DV) −0.5 mm) or VTA (anteroposterior (AP) +3.08 mm, mediolateral (ML) +0.4 mm, dorsoventral (DV) −5.0 mm), and the optical fiber was implanted into the similar location. After 24 h recovery, the mice were anesthetized by an isoflurane anesthesia machine with 2.5% concentration and the head was fixed in a stereotaxic frame. The eyes were protected via coating vet ointment. The FUS transducer was placed on the top of mice head with ultrasound gel filling, where the focal length was set as 1 mm and 5 mm for motor cortex and VTA irradiation via adjusting the transducer water balloon. After that, the FUS pulse (1.55 MPa, 1.5 MHz, 100 ms on 900 ms off) was given, and the photons from the liposomes were collected and recorded via the camera with similar parameters. The relative light power density was obtained according to the calibration curve after correction.

Histology. (a) c-fos staining. Thyl-ChR2-YFP transgenic mice and wild type mice were first treated following the in vivo sono-optogenetics procedures. After 60-90 min, the mice were anesthetized via i.p. injection of ketamine, and perfusion was conducted with PBS and 4% paraformaldehyde. After that, the brain was extracted and stored in 4% paraformaldehyde overnight at 4° C. and sliced through a vibrating blade microtome (Leica VT1200). The brain slices with a depth of 60 μm were washed with 0.3% Triton-X PBS (TBS) solution, and then blocked with 5% bovine serum albumin TBS solution for 30 min at room temperature.

For motor cortex brain slices, after blocking, the solution was replaced by rabbit anti-c-Fos antibody (ab222699, Abcam)/0.3% Triton-X in PBS. The samples were incubated at 4° C. overnight and then washed with TBS solution three times. The mixture of TBS and secondary antibody goat anti-rabbit Alexa Fluor 594 (R37117, Fisher Scientific) and Hoechst 33342 (17535, ATT Bioquest) was added to incubate the slices for 1-2 hours at room temperature in a dark room. Finally, the slices were washed three times with TBS, and then mounted on the slides with mounting media (9990402, Fisher Scientific), and covered with a coverslip. The confocal images were obtained from Zeiss 710 laser scanning microscope.

For VTA brain slices, after blocking, the solution was replaced with mouse anti-c-Fos (E-8) antibody (sc-166940, Santa-Cruz)/rabbit anti-tyrosine hydroxylase antibody (AB152, Sigma-Aldrich)/0.3% Triton-X in PBS. The samples were incubated at 4° C. overnight and then washed with TBS solution three times. The mixture of TBS and secondary antibody goat anti-rabbit Alexa Fluor 594 (R37117, Fisher Scientific) and goat anti-mouse Alexa Fluor 405 (ab175660, Abcam) was added to incubate the slices for 1-2 hours at room temperature in the dark room. Finally, the slices were washed three times with TBS, and then mounted on the slides with mounting media, and covered with a coverslip. The confocal images were obtained from Zeiss 710 laser scanning microscope.

(b) Iba1 staining and Caspase-3 staining. The mice were first treated following the in vivo sono-optogenetics procedures. After 7 days, the mice were anesthetized via i.p. injection of ketamine, and perfusion was conducted with PBS and 4% paraformaldehyde. After that, the brain was extracted and stored in 4% paraformaldehyde overnight at 4° C., and sliced through a vibrating blade microtome. The brain slices with a depth of 60 μm were washed with TBS solution and then blocked with 5% bovine serum albumin TBS solution for 30 min at room temperature. Then, the blocking buffer was replaced by rabbit anti-Iba1 antibody (013-27691, Wako Chem)/TBS or rabbit anti-Cleaved Caspase-3 antibody (9661, Cell Signaling Tec.)/TBS. After incubated overnight at 4° C. fridge, the slices were washed with TBS solution three times, and fresh TBS solution with secondary antibody Donkey anti-rabbit Alexa Fluor 594 (A32754, Invitrogen) and Hoechst 33342 (17535, ATT Bioquest) was added and incubated for 2 h at room temperature in a dark room. Finally, the slices were washed three times with TBS, and then mounted on the slides with mounting media, and covered with a coverslip. The fluorescence images were obtained from a Leica DMi8 fluorescence microscope.

(c) H&E staining. The mice were first treated following the in vivo sono-optogenetics procedures. After 7 days, the mice were anesthetized via i.p. injection of ketamine, and perfusion was conducted with PBS and 4% paraformaldehyde. After that, the brain was extracted and stored in 4% paraformaldehyde overnight at 4° C., and sliced through a vibrating blade microtome. The brain slices with 10 μm depths were obtained for H&E staining according to the protocol (Feldman and Wolfe, 2014). After that, the slices were mounted on the slides with mounting media, and covered with a coverslip. The images were captured with a Nikon Eclipse Ni Compound Light Microscope.

The antibody information was attached in Table 4.

TABLE 4
Antibodies used.

| Primary antibodies | Secondary antibodies |
|---|---|
| Rabbit anti-Iba1 | Donkey anti-Rabbit, Alexa Fluor |
| (1:500, 013-27691, Wako Chemicals) | 594 (1:800, A32754, Invitrogen) |
| Rabbit anti-Cleaved Caspase-3 | Donkey anti-Rabbit, Alexa Fluor |
| (1:500, 9661, Cell Signaling Tec.) | 594 (1:800, A32754, Invitrogen) |
| Anti-Tyrosine Hydroxylase Antibody | Goat anti-Rabbit, Alexa Fluor 594 |
| (1:1000, AB152, Sigma-Aldrich) | (1:500, R37117, Fisher Scientific) |
| Rabbit anti-c-Fos antibody | Goat anti-Rabbit, Alexa Fluor 594 |
| (1:500, ab222699, Abcam) | (1:500, R37117, Fisher Scientific) |
| Mouse anti-c-Fos antibody (E-8) | Goat anti-Mouse, Alexa Fluor 405 |
| (1:500, sc-166940, Santa-Cruz) | (1:500, ab175660, Abcam) |
| | H&E staining kit |
| | (ab245880, Abcam) |
| | Hoechst 33342 |
| | (1:5000, 17535, AAT Bioquest ) |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ahamed et. al., Toxicol. In Vitro 2019, 57, 18.
All et. al., Adv. Mater., 2019, 31, e1803474.
Allen et. al., Adv. Drug Deliv. Rev. 1995, 16, 267.
Baker et. al., Physical Therapy 2001, 81, 1351.
Blackmore et. al., Ultrasound Med. Biol. 2019, 45, 1509.
Bobo et. al., Pharm. Res. 2016, 33, 2373.
Bottomley and Andrew, Phys. Med. Biol. 1978, 23, 630.
Boyden et. al., Nat. Neurosci. 2005, 8, 1263.
Cadoni et. al., bioRxiv 2021, 2021.11.07.467597.
Cadoni et. al., Nat. Nanotechnol. 2023, DOI 10.1038/s41565-023-01359-6.
Cai and Tong, Front. Neural Circuits 2022, 16, 867053.
Chang et. al., Ultrason. Sonochem. 2019, 53, 59.
Chen et. al., ACS Nano 2021, 15, 5201.
Chen et. al., Nat. Biotechnol. 2020, 39, 161.
Chen et. al., Nat. Biotechnol. 2021, 39, 161.
Chen et. al.,Science 2015, 347, 1477.
Chen et. al., Science 2018, 359, 679.
Chen et. al., Science 2018, 359, 679.
Choi et. al., Bioconjug. Chem. 2020, 31, 967.
Daiber et. al., Free Radic. Res. 2004, 38, 259.
Deisseroth, Nat. Neurosci. 2015, 18, 1213.
Deisseroth, Nat. Neurosci., 2015, 18, 1213, 2015.
Duco et. al., Methods 2016, 109, 141.
Duque et. al., Nat. Commun. 2022, 13, 600.
Emiliani et. al., Nature Reviews Methods Primers, 2022, 2, 1.
Entradas et. al., J. Photochem. Photobiol. B 2020, 204, 111787.
Etter et. al., Nat. Commun. 2019, 10, 5322.
Etter et. al., Nat. Commun. 2019, 10, 5322.
Feldman and Wolfe, Methods Mol. Biol. 2014, 1180, 31.
Finlayson et. al., Photochem. Photobiol. 2022, 98, 974.
Fougère et. al., Proc. Natl. Acad. Sci. U.S.A. 2021, 118, DOI 10.1073/pnas.2110934118.
Gomes et. al., J. Biochem. Biophys. Methods 2005, 65, 45.
Gomes et. al., J. Biochem. Biophys. Methods 2005, 65, 45.
Gong et. al., Neuron 2020, 107, 197.
Hochbaum et. al., Nat. Methods 2014, 11, 825.
Hochbaum et. al., Nat. Methods 2014, 11, 825.
Hochbaum et. al., Nat. Methods 2014, 11, 825.
Holt and Roy, Ultrasound Med. Biol. 2001, 27, 1399.
Hong, Science 2020, 369, 638.
Hong, Science 2020, 369, 638.
Huang et. al., Nano Lett. 2020, 20, 1089.
Ibsen et. al., Nat. Commun. 2015, 6, 8264.
Inoue et. al., Cell 2019, 177, 1346.
Jiang et. al., Chemistry 2021, 27, 12589.
Jiang et. al., Chemistry 2021, 27, 12589.
Jiang et. al., Chemistry 2021, 27, 12589.
Jiménez-Pérez et. al., Sensors and Actuators B: Chemical 2019, 298, 126878.
Kahlon et. al., Environ. Chem. Lett. 2018, 16, 919.
Kim et. al., Nat. Rev. Neurosci. 2017, 18, 222.
Kim et. al., Proc. Natl. Acad. Sci. U.S.A., 2019, 116, 10214.
Kim et. al., Proc. Natl. Acad. Sci. U.S.A., 2022, 119, DOI 10.1073/pnas.2109791119.
Klapoetke et. al., Nat. Methods 2014, 11, 338.
Kubanek et. al., J. Neurosci. 2018, 38, 3081.
Lee et. al., Nat. Mater. 2021, 20, 1029.
Li et. al., Nanoscale 2022, 14, 2277.
Li et. al., ACS Nano 2016.
Liu et. al., Brain Stimul. 2022, 15, 1308.
Liu et. al., Small 2017, 13, 1701621.
Liu et. al., Small 2017, 13, 1701621.
Maresca et. al., Annu. Rev. Chem. Biomol. Eng. 2018, 9, 229.
Marshel et. al., Science 2019, 365, DOI 10.1126/science.aaw5202.
Martorell et. al., Cell 2019, 177, 256.
Matsubara et. al., n.d., DOI 10.1101/798702.
Meng et. al., Nat. Rev. Neurol. 2021, 17, 7.
Merényi et. al., J. Biolumin. Chemilumin. 1990, 5, 53.
Mittler, Trends Plant Sci. 2017, 22, 11.
Nath et. al., Nat. Protoc. 2019, 14, 2152.
Nieuwenhuis et. al., Gene Ther. 2021, 28, 56.
Nosova et al., Medchemcomm. 2019 Feb 12; 10 (3): 369-377.
Ou et. al., Nature 2021, 590, 410.
Ouyang et. al., Nano Today 2020, 35, 100949.
Packer et. al., Nat. Methods 2012, 9, 1202.
Pan et. al., Adv. Mater. 2018, 30, 1800180.
Pei et. al., Nat. Nanotechnol. 2021, 16, 1011.
Qiu et. al., Cell Rep. 2020, 32, 108033.
Rao et. al., Nat. Nanotechnol. 2019, 14, 967.
Romero et. al., Nature Reviews Methods Primers 2022, 2, 1.
Rwei et. al., Nat Biomed Eng 2017, 1, 644.
Salatino et. al., Nat Biomed Eng 2017, 1, 862.
Sanes and Donoghue, Annu. Rev. Neurosci. 2000, 23, 393.
Sato et. al., Neuron 2018, 98, 1031.
Schoenmaker et. al., Int. J. Pharm. 2021, 601, 120586.
Sebesta et. al., Nat. Mater. 2022, DOI 10.1038/s41563-022-01281-7.
Soenen et. al., Nano Today 2011, 6, 446.
Stauffer and Paulides, in Comprehensive Biomedical Physics (Ed.: A. Brahme), Elsevier, Oxford, 2014, pp. 115-151.
Tsoi et. al., Nat. Mater. 2016, 15, 1212.
Tyrrell et. al., Biochim. Biophys. Acta 1976, 457, 259.
Vernon and Lewin, in Avery's Diseases of the Newborn (Ninth Edition) (Eds.: C. A. Gleason, S. U. Devaskar), W.B. Saunders, Philadelpia, 2012, pp. 741-750.
Vogt, Nat. Methods 2016, 13, 468.
Wang and Liu, Nanomaterials and Neoplasms 2021, 847.
Wang and Yi, Cancer Biol. Ther. 2008, 7, 1875.
Wang et. al., ACS Appl. Mater. Interfaces 2017, 9, 15986.
Wang et. al., ACS Appl. Mater. Interfaces 2018, 10, 19398.
Wang et. al., ACS Appl. Mater. Interfaces 2018, 10, 19398.
Wang et. al., Adv. Drug Deliv. Rev., 2022, 186, 114343.
Wang et. al., Biomacromolecules 2016, 17, 2223.
Wang et. al., Chem. Mater. 2021, 33, 774.
Wang et. al., Chem. Mater. 2021, 33, 774.
Wang et. al., Chem. Mater. 2021, 33, 774.
Wang et. al., Chem. Mater. 2021, 33, 774.
Wang et. al., J. Am. Chem. Soc. 2023, 145, 1097.
Wang et. al., J. Am. Chem. Soc. 2023, 145, 1097.
Wang et. al., Neuron 2018, 100, 728.
Won et. al., Cell 2020, 181, 115.
Wu et. al., Nat Biomed Eng 2022, 6, 754.
Wu et. al., Proc. Natl. Acad. Sci. U.S.A. 2019, 116, 26332.
Wu et. al., Proc. Natl. Acad. Sci. U.S.A. 2019, DOI 10.1073/pnas. 1914387116.
Xu et. al., bioRxiv 2022, 2022.10.28.514307.
Xu et. al., Nat Biomed Eng., 2022, DOI 10.1038/s41551-022-00978-z.
Yamaguchi et. al., Anal. Chim. Acta 2010, 665, 74.
Yang and Kwan, Int. Rev. Neurobiol. 2021, 158, 443.
Yang et. al., Adv. Drug Deliv. Rev. 2023, 194, 114711.
Yang et. al., J. Am. Chem. Soc., 2022, 144, 18406.
Yang et. al., Sci Adv., 2022, 8, eabo6743.
Yizhar et. al., Nature 2011, 477, 171.
Yu et. al., Nat. Commun. 2021, 12, 2519.
Zhang et. al., ACS Appl. Mater. Interfaces 2019, 11, 9355.
Zhang et. al., Nature 2007, 446, 633.
Zhang et. al., Nature 2007, 446, 633.
Zhang et. al., Nature 2007, 446, 633.
Zhang et. al., Nature 2007, 446, 633.

1. A pharmaceutical composition comprising:
(i) liposomes or nanoparticles, wherein the liposomes or nanoparticles comprise a sonosensitizer and a chemiluminescent compound; and
(ii) an excipient.

2. The pharmaceutical composition of claim 1, wherein the nanoparticles are a biocompatible, biodegradable organic nanoparticles.

3. The pharmaceutical composition of claim 2, wherein the nanoparticle is a lipid nanoparticle.

4. The pharmaceutical composition of claim 1, wherein the sonosensitizer produces free radicals or a reactive oxygen species (ROS) in response to ultrasound stimulation.

5. The pharmaceutical composition of claim 1, wherein the chemiluminescent compound emits light in response to free radicals or a reactive oxygen species (ROS).

6. The pharmaceutical composition of claim 5, wherein the reactive oxygen species (ROS) is singlet oxygen (¹O₂) or hydroxyl radical (·OH).

7. The pharmaceutical composition of claim 1, wherein the sonosensitizer is IR780, or DCPH-P-Na (I), Hematoporphyrin, Zinc protoporphyrin, methylene blue, TiO₂, Chlorin e6.

8. (canceled)

9. The pharmaceutical composition of claim 7, wherein the chemiluminescent compound is L012, a dioxetane, luminol, isoluminol, an imidazopyrazinone, a lophine, or an acridinium.

10. (canceled)

11. The pharmaceutical composition of claim 1, wherein the sonosensitizer is IR780 and wherein the chemiluminescent compound is L012.

12. The pharmaceutical composition of claim 1, wherein the sonosensitizer is IR780, DCPH-P-Na (I), Hematoporphyrin, Zinc protoporphyrin or methylene blue, TiO₂, Chlorin e6 and wherein the chemiluminescent compound is L012, a Dioxetane derivative, luminol, isoluminol, a imidazopyrazinone derivative, a lophine derivative or an acridinium derivative.

13. The pharmaceutical composition of claim 1, wherein the nanoparticle or liposome is about 10-1000 nm in diameter.

14-15. (canceled)

16. The pharmaceutical composition of claim 1, wherein the liposomes or nanoparticles comprise a phospholipid, 1,2-distearoryl-sn-glycero-3-phosphatidyl choline (DSPC), sphingomyelin, phosphatidylcholine (e.g., egg phosphatidylcholine or soy phosphatidylcholine), monosialoganglioside, cholesterol, polyethylene glycol (PEG), PEG-succinyl cysteine (PEG-SC), poly (lactic-co-glycolic acid) (PLGA), dioleoylphosphatidylethanolamine (DOPE), 1,2-dipalmitoyl-sn-glycero-3-phosphocholine (DPPC), cholesterol, 1,2-distearoyl-sn-glycero-3-phosphoethanolamine-N-[amino(polyethylene glycol)](DSPE-PEG), or choline phosphate.

17. The pharmaceutical composition of claim 16, wherein the liposomes or nanoparticles comprise a polyethylene glycol.

18. (canceled)

19. The pharmaceutical composition of claim 17, wherein the polyethylene glycol is PEG-100, PEG-150, PEG-200, PEG-250, PEG-300, PEG-350, PEG-400, PEG-450, or PEG-500.

20. The pharmaceutical composition of claim 17 wherein the polyethylene glycol is present in the liposome or nanoparticle in an amount of about 1-10 (w/w) % or about 0.1-5 mol. %.

21. The pharmaceutical composition of claim 16, wherein the liposomes or nanoparticles comprise 1,2-dipalmitoyl-sn-glycero-3-phosphocholine (DPPC), cholesterol, and 1,2-distearoyl-sn-glycero-3-phosphoethanolamine-N-[amino(polyethylene glycol)-2000] (DSPE-PEG₂₀₀₀).

22-30. (canceled)

31. The pharmaceutical composition of claim 1, wherein the nanoparticle or liposome comprises calcium peroxide (CaO₂) and a polyethylene glycol (PEG).

32. (canceled)

33. A method of contacting a tissue of a subject with light, comprising: applying an ultrasound signal to a nanoparticle or liposome in proximity to the tissue of the subject, wherein the nanoparticle or liposome comprises a photosensitizer and a chemiluminescent compound, and wherein the ultrasound signal causes the photosensitizer to induce the chemiluminescent to emit light that contacts the tissue.

34-84. (canceled)

85. A system for contacting a tissue of a subject with light, the system comprising: an ultrasound device configured to apply an ultrasound signal to a nanoparticle or liposome of claim 1 in proximity to the tissue, thereby causing the mechanoluminescent particle to emit light that contacts the tissue.

86. (canceled)

87. A kit comprising the liposome or nanoparticle of claim 1 in a container.

88. (canceled)
